(19) United States
(12) Patent Application Publication
(10) Pub. No.: US 2026/0021182 A1
(43) Pub. Date: Jan. 22, 2026

(54) A MOLECULAR SWITCH TO TUNE THE AVIDITY OF IMMUNE CELLS TO THEIR TARGET

(71) Applicant: Baylor College of Medicine, Houston, TX (US)

(72) Inventors: Nabil M. Ahmed, Houston, TX (US); Ahmed Gad, Houston, TX (US); Jessica Morris, Houston, TX (US); Sujith Joseph, Houston, TX (US)

(73) Assignee: Baylor College of Medicine, Houston, TX (US)

(21) Appl. No.: 19/133,431

(22) PCT Filed: Dec. 6, 2023
(86) PCT No.: PCT/US2023/082707

§ 371 Date: May 28, 2025

Related U.S. Application Data
(60) Provisional application No. 63/430,982, filed on Dec. 7, 2022.

(51) Int. Cl. A61K 40/11; A61K 40/31; A61K 40/42; C07K 14/725; C07K 16/28; C07K 16/32

(52) U.S. Cl. CPC A61K 40/11; A61K 40/31; A61K 40/4205; A61K 40/421; C07K 14/7051; C07K 16/2818; C07K 16/32; A61K 2239/23

(57) ABSTRACT

Disclosed herein, in some aspects, are engineered avidity switch polypeptides, polynucleotides encoding the same, immune cells comprising the same, and compositions comprising the polypeptides, polynucleotides, and/or immune cells. In certain embodiments, engineered avidity switch polypeptides comprise one or more CD6 derived amino acid sequences and/or domains. In certain embodiments, engineered avidity switch polypeptides comprise engineered arrangements of extracellular domains of CD6 polypeptides. Also disclosed are methods for disease treatment, such as cancer treatment, comprising administering such immune cells and/or compositions to a subject in need thereof.

### CROSS-REFERENCE TO RELATED APPLICATIONS

This application claims the benefit of priority of U.S. Provisional Patent Application No. 63/430,982 filed Dec. 7, 2022, which is hereby incorporated by reference in its entirety.

### STATEMENT OF GOVERNMENT SUPPORT

This invention was made with government support under U54 CA232568-01 awarded by the National Institutes of Health. The government has certain rights in the invention.

### SEQUENCE LISTING

The instant application contains a Sequence Listing which has been submitted in ST26 format and is hereby incorporated by reference in its entirety. Said ST26 copy, created on Nov. 30, 2023, is named BAYM_P0376WO_Sequence_Listing.xml and is 87,969 bytes in size.

### TECHNICAL FIELD AND BACKGROUND

### I. Technical Field

Aspects of this disclosure relate to at least the fields of immunology, immunotherapeutics, cancer biology, and medicine.

### II. Background

Immune cell therapies are biotherapies currently approved for the treatment of some relapse/refractory malignancies and are promising with nonmalignant conditions. Despite their successes, not all patients respond to treatment. In addition, application of these therapies to certain entities, such as solid malignancies, have proven to be more difficult. Challenges to overcome include maintaining immune cell activation and proliferation and preventing therapeutic cell exhaustion. Current approaches to enhance immune cell activation have adapted various costimulatory molecules in effector cells, such as T cells, resulting in, for example, the development of chimeric antigen receptor (CAR) T cells with rapid responses in vitro but lacking the ability to maintain initial activation and persistence in vivo.

There exists a need for improved compositions and methods for cellular immunotherapy.

### SUMMARY

Aspects of the present disclosure address certain needs by providing at least nucleic acids, cells, proteins, and methods useful in immunotherapy. Aspects of the disclosure include nucleic acids encoding an engineered polypeptide that can alter (e.g., increase or decrease) the avidity of an immune cell, referred to herein as an avidity switch polypeptide (e.g., an avidity switch, such as an avidity switch comprising components of a CD6 polypeptide). Further aspects are directed to immune cells of any kind (e.g., T cells, NK cells, NKT cells, transgenic TCR (tgTCR) cells (of any kind, including T cells, NK cells, and/or NKT cells), cytotoxic T lymphocyte, expressing an engineered avidity switch polypeptide, and optionally expressing an engineered antigen receptor (e.g., a CAR, a TCR, an antibody, etc.), as well as methods of use of such immune cells for treatment of a disease, such as but not limited to an autoimmune disease, an infection, a neurological disorder, a neurodegenerative disorder, and/or cancer.

In certain embodiments, a polynucleotide encoding an engineered avidity switch polypeptide to increase immune cell avidity is provided. In certain embodiments, the engineered avidity switch comprises an extracellular stabilization domain encoding sequence, a Scavenger Receptor Cysteine-Rich (SRCR) domain encoding sequence, a hinge region encoding sequence, a transmembrane domain encoding sequence, and/or an intracellular signaling region encoding sequence. In certain embodiments, the extracellular stabilization domain encoding sequence comprises, consists essentially of, or consists of a Cluster of Differentiation 6 (CD6) N-terminal encoding sequence at least or exactly 70%, 75%, 80%, 85%, 90%, 95%, 98%, or 100% identical to SEQ ID NOs: 47 or 48. In certain embodiments, the SRCR domain encoding sequence comprises, consists essentially of, or consists of a CD6 extracellular domain encoding sequence. In certain embodiments, the hinge region encoding sequence comprises, consists essentially of, or consists of a CD6 hinge region encoding sequence at least or exactly 70%, 75%, 80%, 85%, 90%, 95%, 98%, or 100% identical to SEQ ID NOs: 41 or 42. In certain embodiments, the transmembrane domain encoding sequence comprises, consists essentially of, or consists of a CD6 transmembrane region encoding sequence at least or exactly 70%, 75%, 80%, 85%, 90%, 95%, 98%, or 100% identical to SEQ ID NOs: 43-46. In certain embodiments, the intracellular signaling region encoding sequence comprises, consists essentially of, or consists of a CD6 intracellular signaling region encoding sequence at least or exactly 70%, 75%, 80%, 85%, 90%, 95%, 98%, or 100% identical to SEQ ID NOs: 28-36. In certain embodiments, the polynucleotide further comprises a signal peptide encoding sequence that comprises, consists of, or consists essentially of a CD6 signal peptide encoding sequenced at least or exactly 70%, 75%, 80%, 85%, 90%, 95%, 98%, or 100% identical to SEQ ID NOs: 37 or 38. In certain embodiments, the polynucleotide comprises a CD6 extracellular domain 2 (d2) and extracellular domain 3 (d3) linker region encoding sequence that comprises, consists essentially of, or consists of a CD6 d2-d3 linker region encoding sequence at least or exactly 70%, 75%, 80%, 85%, 90%, 95%, 98%, or 100% identical to SEQ ID NO: 49. In certain embodiments, the polynucleotide may further comprise one or more, non-CD6, hinge region encoding sequence, for example but not limited to, a cluster of differentiation 4 (CD4) and/or an Immunoglobulin G 1 (IgG1) hinge encoding sequence. In certain embodiments, the SRCR domain comprises, consists essentially of, or consists of one or more CD6 SRCR domains, for example but not limited to domain 3 (d3). In certain embodiments, one or more encoding sequences are operably linked in tandem, optionally in the 5′ to 3′ orientation of: i) an extracellular stabilization domain encoding sequence, ii) an SRCR domain encoding sequence, iii) a hinge region encoding sequence, iv) a transmembrane domain encoding sequence, and v) an intracellular signaling region encoding sequence. In some embodiments, the polynucleotide is operably linked to a regulatory sequence comprising one or more of a promoter, a transcription initiation sequence, an internal ribosome entry site (IRES), an enhancer, an intron, an RNA interference target sequence, a Kozak sequence, splicing regulatory elements, and/or a polyadenylation signal. In some embodiments, the polynucleotide is comprised in a vector, e.g. but not limited to a plasmid, a recombinant vector, and/or a viral vector. In some embodiments, polynucleotide is inserted into a genomic locus, and incorporated as part of the genome of a target cell. In some embodiments, incorporation into the genome of a cell comprises use of guided endonucleases, such as CRISPR/Cas systems. In some embodiments, incorporation into the genome of a cell comprises use of a recombinase. In some embodiments, the viral vector comprises a retrovirus, lentivirus, adenovirus, and/or adeno-associated virus.

In some embodiments, methods for making a transgenic avid cell expressing one or more recombinant proteins is provided. In certain embodiments, the method comprises transfecting, transforming, and/or transducing a cell in vitro and/or ex vivo with a polynucleotide expressing an engineered avidity switch polypeptide. In certain embodiments, the transgenic avid cell is a mammalian cell, e.g. but not limited to mouse and/or human cell. In some embodiments, the mammalian cell is an immune cell, e.g. but not limited to a T cell, natural killer T cell (NK T cell), macrophage, dendritic cell, cytotoxic T lymphocyte, tumor-infiltrating lymphocyte, and/or natural killer (NK) cell. In some embodiments the immune cell is an autologous or allogenic cell. In some embodiments, the immune cell comprises one or more mutations in an endogenous gene associated with immune suppression and/or immune activation. In some embodiments, the immune cell expresses one or more additional transgenes. In some embodiments, an additional transgene can comprise one or more antigen recognition domain encoding sequences, such as but not limited to, an antibody, antibody derivative, chimeric antigen receptor (CAR) and/or T-cell receptor (TCR). In some embodiments, a CAR and/or TCR targets a Human Epidermal Growth Factor Receptor 2 (Her2) and/or Cluster of Differentiation 19 (CD19) antigen. In some embodiments, the CAR comprises a HER2.CD2ζ and/or a CD19.41BBζ CAR. In some embodiments, the transgenic avid cell exhibits a greater than two-fold, four-fold, six-fold, eight-fold, and/or ten-fold greater avidity to a target comprising a target antigen relative to a control cell. In some embodiments, the transgenic avid cell exhibits an increase in leukocyte adhesion glycoprotein 1 (LFA-1) recruitment to an immune synapse relative to a control cell. In certain embodiments, the transgenic avid cell exhibits an increase in activated LFA-1 levels relative to a control cell. In certain embodiments, the transgenic avid cell exhibits greater killing efficiency of a target cell relative to a control cell. In certain embodiments, the transgenic avidity reduced cell exhibits a reduced killing efficiency of a target cell relative to a control cell. In certain embodiments, the transgenic avid cell has a manipulated level of avidity that is at or near the threshold required for cell activation. In certain embodiments, the transgenic avid cell comprises one or more mutations in one or more endogenous genes. In some embodiments, the control cell is a non-transgenic cell, and/or a non-avid transgenic cell. Advantageously, in certain embodiments, compositions described herein, when expressed by an immune effector cell, can increase the avidity of an immune cell to its target.

In some embodiments, a composition comprising the polynucleotide, polypeptide, and/or transgenic avid cell is provided. In some embodiments, the composition further comprises a pharmaceutically acceptable carrier.

In some embodiments, disclosed herein are methods for treating an individual comprising administering to the individual in need thereof, a polynucleotide, polypeptide, transgenic avid cell, and/or composition described herein, is provided. In some embodiments, the individual has, is diagnosed with, and/or is suspected of having: cancer, an autoimmune disorder, a viral infection, neurological disorder, neurodegenerative disease, and/or a bacterial infection. In some embodiments, the individual has, is diagnosed with, and/or is suspected of having cancer that has a modest and/or low tumor associated antigen (TAA) expression level relative to other cancers expressing the same TAA, e.g. but not limited to glioblastoma, medulloblastoma, and/or glioblastoma multiform. In some embodiments the patient has been diagnosed with a cancer having modest or low Her2 TAA expression relative to other cancers expressing Her2 TAA.

In certain embodiments, provided herein is a polynucleotide encoding an engineered avidity switch polypeptide designed to decrease immune cell avidity. In some embodiments, an engineered avidity switch designed to decrease immune cell avidity comprises an extracellular stabilization domain encoding sequence, an extracellular domain encoding sequence, a transmembrane domain encoding sequence, and an intracellular signaling region encoding sequence, wherein the polynucleotide is operably linked to a non-endogenous promoter sequence. In certain embodiments, the extracellular stabilization domain encoding sequence comprises, consists essentially of, or consists of a sequence at least or exactly 70%, 75%, 80%, 85%, 90%, 95%, 98%, or 100% identical to SEQ ID NO: 47 or 48. In certain embodiments, the one or more extracellular domain encoding sequence comprises, consists of, or consists essentially of a CD6 extracellular domain 1 (d1) encoding sequence at least or exactly 70%, 75%, 80%, 85%, 90%, 95%, 98%, or 100% identical to SEQ ID NO: 53, a CD6 extracellular domain 2 (d2) encoding sequence at least or exactly 70%, 75%, 80%, 85%, 90%, 95%, 98%, or 100% identical to SEQ ID NO: 55, and/or a CD6 extracellular domain 3 (d3) encoding sequence at least or exactly 70%, 75%, 80%, 85%, 90%, 95%, 98%, or 100% identical to SEQ ID NOs: 39 or 40. In certain embodiments, the one or more extracellular domain encoding sequences comprise, consist essentially of, and/or consists of a sequence encoding CD6 d1, and/or d2 sequence at least or exactly 70%, 75%, 80%, 85%, 90%, 95%, 98%, or 100% identical to SEQ ID NOs: 53 and 55 respectively, and does not comprise a sequence encoding CD6 d3. In some embodiments the d1, d2, and/or d3 domains comprise engineered mutations to alter the independent and/or combined functions of these domains. In certain embodiments, the transmembrane domain encoding sequence comprises, consists essentially of, and/or consists of a CD6 transmembrane region encoding sequence at least or exactly 70%, 75%, 80%, 85%, 90%, 95%, 98%, or 100% identical to SEQ ID NOs: 43-46. In certain embodiments, the intracellular signaling region encoding sequence comprises, consists essentially of, and/or consists of a CD6 intracellular signaling region encoding sequence at least or exactly 70%, 75%, 80%, 85%, 90%, 95%, 98%, or 100% identical to SEQ ID NOs: 28-36. In certain embodiments, the CD6 intracellular signaling region has been genetically engineered to modify the function of the CD6 intracellular signaling region relative to wild type full length CD6 protein. In certain embodiments the encoding sequences are operably linked in tandem, optionally comprising a 5′ to 3′ orientation of: i) extracellular stabilization domain encoding sequence, ii) one or more extracellular domain encoding sequence(s), iii) transmembrane domain encoding sequence, and iv) an intracellular signaling region encoding sequence. In some embodiments, the polynucleotide comprises a nucleic acid sequence encoding a CD6 hinge region that comprises, consists essentially of, and/or consists of a sequence at least or exactly 70%, 75%, 80%, 85%, 90%, 95%, 98%, or 100% identical to SEQ ID NOs: 41 or 42, interspersed between the one or more extracellular domain encoding sequences and the transmembrane domain encoding sequence. In some embodiments, the polynucleotide comprises a nucleic acid sequence encoding a CD6 ECS-D1 linker (SEQ ID NO: 52) interspersed between the extracellular stabilization domain and a first extracellular domain, and a nucleic acid sequence encoding a CD6 D1-D2 linker (SEQ ID NO: 54) comprised between two extracellular domain encoding sequences. In some embodiments, the polynucleotide further comprises a signal peptide encoding sequence, wherein the signal peptide encoding sequence comprises, consists essentially of, or consists of a sequence at least or exactly 70%, 75%, 80%, 85%, 90%, 95%, 98%, or 100% identical to SEQ ID NOs: 37 or 38. In some embodiments, the polynucleotide is operably linked to a regulatory sequence comprising one or more of, a transcription initiation sequence, an internal ribosome entry site (IRES), an enhancer, an intron, an RNA interference target sequence, a Kozak sequence, splicing regulatory elements, and/or a polyadenylation signal. In some embodiments, the polynucleotide comprised in a vector, e.g. but not limited to a plasmid, recombinant vector, and/or a viral vector. In some embodiments, the viral vector comprises a retroviral, lentiviral, adenoviral, or adeno-associated viral packaging vector.

In some embodiments, provided herein are polypeptides comprising an extracellular stabilization domain that comprises, consists essentially of, or consists of a sequence at least or exactly 70%, 75%, 80%, 85%, 90%, 95%, 98%, or 100% identical to SEQ ID NO: 13, one or more extracellular domains, a transmembrane domain, and an intracellular signaling region. In some embodiments, the one or more extracellular domains comprises, consists essentially of, or consists of a sequence at least or exactly 70%, 75%, 80%, 85%, 90%, 95%, 98%, or 100% identical to SEQ ID NOs: 18, 20, and/or 10. In some embodiments, the one or more extracellular domains comprises a CD6 extracellular domain 1 (d1) amino acid sequence comprising, consisting essentially of, or consisting of an amino acid sequence at least or exactly 70%, 75%, 80%, 85%, 90%, 95%, 98%, or 100% identical to SEQ ID NO: 18, and a CD6 extracellular domain 2 (d2) amino acid sequence comprising, consisting essentially of, or consisting of an amino acid sequence at least or exactly 70%, 75%, 80%, 85%, 90%, 95%, 98%, or 100% identical to SEQ ID NO: 20, and does not comprise a CD6 extracellular domain 3 (d3) amino acid sequence. In some embodiments, a polypeptide comprising d1, d2, and/or d3 comprises engineered mutations that modify the independent and/or combined endogenous functions of d1, d2, and/or d3 while retaining at least or exactly 70%, 75%, 80%, 85%, 90%, 95%, 98%, or 100% sequence identity to SEQ ID NOs: 18, 20, and/or 10. In some embodiments, the transmembrane domain comprises a CD6 transmembrane region comprising, consisting essentially of, or consisting of a sequence at least or exactly 70%, 75%, 80%, 85%, 90%, 95%, 98%, or 100% identical to SEQ ID NO: 12. In some embodiments, the intracellular signaling region comprises a CD6 intracellular signaling sequence comprising, consisting essentially of, or consisting of a sequence at least or exactly 70%, 75%, 80%, 85%, 90%, 95%, 98%, or 100% identical to SEQ ID NO: 2-8. In some embodiments, the CD6 intracellular signaling region comprises genetically engineered mutations modifying the function of the CD6 intracellular signaling region relative to endogenous functions. In some embodiments, the polypeptide comprises domains and/or regions operably linked in tandem. In some embodiments, the polypeptide comprises an N- to C-terminus orientation of: i) the extracellular stabilization domain, ii) the one or more extracellular domain(s), iii) the transmembrane domain, and viii) and the intracellular signaling region. In some embodiments, the polypeptide comprises a CD6 hinge region sequence comprising, consisting essentially of, or consisting of a sequence at least or exactly 70%, 75%, 80%, 85%, 90%, 95%, 98%, or 100% identical to SEQ ID NO: 11, interspersed between the one or more extracellular domains and the transmembrane domain. In some embodiments, the polypeptide comprises a CD6 ECS-D1 linker sequence (SEQ ID NO: 17) interspersed between the extracellular stabilization domain and a first extracellular domain, and a CD6 D1-D2 linker sequence (SEQ ID NO: 19) comprised between two extracellular domains. In some embodiments, the polypeptide comprises a signal peptide sequence, wherein the signal peptide sequence comprises, consists essentially of, or consists of a sequence at least or exactly 70%, 75%, 80%, 85%, 90%, 95%, 98%, or 100% identical to SEQ ID NO: 9. In some embodiments, the polypeptide comprises, consists essentially of, or consists of an amino acid at least or exactly 70%, 75%, 80%, 85%, 90%, 95%, 98%, or 100% identical to SEQ ID NO: 25.

In some embodiments, provided herein are methods for making a transgenic avidity-weakened cell expressing one or more recombinant proteins. In certain embodiments, the method comprises transfecting, transforming, and/or transducing a cell in vitro and/or ex vivo with the polynucleotide expressing an engineered avidity switch polypeptide. In certain embodiments, the transgenic avid cell is a mammalian cell, e.g. but not limited to mouse and/or human cell. In some embodiments, the mammalian cell is an immune cell, e.g. but not limited to a T cell, natural killer T cell (NK T cell), macrophage, dendritic cell, tumor infiltrating lymphocyte, cytotoxic T lymphocyte, and/or natural killer (NK) cell. In some embodiments the immune cell is an autologous or allogenic cell. In some embodiments, the immune cell comprises one or more mutations in an endogenous gene associated with immune suppression and/or immune activation. In some embodiments, the immune cell expresses one or more additional transgenes comprising an antigen recognition domain encoding an antibody, antibody derivative, chimeric antigen receptor (CAR) and/or T-cell receptor (TCR). In some embodiments, the CAR and/or TCR targets a Human Epidermal Growth Factor Receptor 2 (Her2) and/or Cluster of Differentiation 19 (CD19) antigen. In some embodiments, the CAR comprises a HER2.CD28ζ and/or a CD19.41BBζ CAR. In some embodiments, the transgenic avid cell exhibits a greater than two-fold decrease in avidity compared to a control cell. In some embodiments, the control cell is a transgenic non-avidity-weakened cell, non-transgenic cell, and/or an avid transgenic cell. In some embodiments, the transgenic avid cell exhibits a decrease in leukocyte adhesion glycoprotein 1 (LFA-1) recruitment to an immune synapse relative to a control cell. In certain embodiments, the transgenic avid cell exhibits a decrease in activated LFA-1 levels relative to a control cell. In certain embodiments, the transgenic avidity-weakened cell exhibits greater killing efficiency of a target cell relative to a control cell. In certain embodiments, the transgenic avidity-weakened cell comprises one or more mutations in one or more endogenous genes. Advantageously, these compositions, when expressed by an immune effector cell, can decrease the avidity of an immune cell to its target.

In some embodiments, a composition comprising the polynucleotide, polypeptide, and/or transgenic avidity-weakened cell is provided. In some embodiments, the composition further comprises a pharmaceutically acceptable carrier.

In some embodiments, provided herein are methods for treating an individual comprising administering to the individual in need thereof, a polynucleotide, polypeptide, transgenic avidity-weakened cell, and/or composition described herein. In some embodiments, the individual has, is diagnosed with, and/or is suspected of having: cancer, an autoimmune disorder, a viral infection, neurological disorder, neurodegenerative disorder, and/or a bacterial infection. In some embodiments, the individual has, is diagnosed with, and/or is suspected of having cancer that has a medium and/or high tumor associated antigen (TAA) expression level relative to other cancers expressing the same TAA, e.g. but not limited to glioblastoma, medulloblastoma, and/or glioblastoma multiform. In some embodiments the patient has been diagnosed with a cancer having medium and/or high Her2 TAA expression relative to other cancers expressing Her2 TAA.

Certain embodiments of the present invention are characterized through the following enumerated aspects.

Aspect 1, is a polynucleotide encoding an engineered avidity switch, comprising an extracellular stabilization domain encoding sequence comprising at least about 80% sequence identity to SEQ ID NOs: 47 or 48, a Scavenger Receptor Cysteine-Rich (SRCR) domain encoding sequence comprising at least about 80% sequence identity to SEQ ID NOs: 39 or 40, a hinge region encoding sequence, a transmembrane domain encoding sequence, and an intracellular signaling region encoding sequence.

Aspect 2, is polynucleotide of aspect 1, further comprising a Cluster of Differentiation 6 (CD6) extracellular domain 2 (d2) and domain 3 (d3) linker region encoding sequence comprising at least about 80% sequence identity to SEQ ID NO: 49.

Aspect 3, is the polynucleotide of aspect 1 or 2, wherein the hinge region encoding sequence comprises a CD6 hinge region encoding sequence comprising at least about 80% sequence identity to SEQ ID NOs: 41 or 42.

Aspect 4, is the polynucleotide of any one of aspects 1 to 3, wherein the transmembrane domain encoding sequence comprises a CD6 transmembrane region encoding sequence comprising at least about 80% sequence identity to SEQ ID NOs: 43-46.

Aspect 5, is the polynucleotide of any one of aspects 1 to 4, wherein the intracellular signaling region encoding sequence comprises a CD6 intracellular signaling region encoding sequence comprising at least about 80% sequence identity to SEQ ID NOs: 28-36.

Aspect 6, is the polynucleotide of any one of aspects 1 to 5, further comprising a signal peptide encoding sequence, wherein optionally the signal peptide encoding sequence comprises a sequence at least about 80% identical to SEQ ID NOs: 37 or 38.

Aspect 7, is the polynucleotide of any one of aspects 1 to 6, further comprising a non-CD6 hinge region encoding sequence.

Aspect 8, is the polynucleotide of aspect 7, wherein the non-CD6 hinge region encoding sequence comprises a Cluster of Differentiation 4 (CD4) and/or an Immunoglobulin G 1 (IgG1) hinge encoding sequence comprising at least about 80% sequence identity to SEQ ID NOs: 50 and/or 51.

Aspect 9, is the polynucleotide of any one of aspects 1 to 8, wherein the an extracellular stabilization domain encoding sequence comprises a CD6 extracellular stabilization (ECS) domain encoding sequence comprising at least about 90% sequence identity to SEQ ID NO: 47 or 48.

Aspect 10, is the polynucleotide of any one of aspects 1 to 9, wherein the SRCR domain encoding sequence comprises a CD6 SRCR domain encoding sequence comprising at least about 90% identity to SEQ ID NO: 39 or 40.

Aspect 11, is the polynucleotide of any one of aspects 1 to 10, wherein the encoding sequences are operably linked in tandem.

Aspect 12, is the polynucleotide of any one of aspects 1 to 11, comprising a 5′ to 3′ orientation of: i) the extracellular stabilization domain encoding sequence, ii) the SRCR domain encoding sequence, iii) the hinge region encoding sequence, iv) the transmembrane domain encoding sequence, and v) the intracellular signaling region encoding sequence.

Aspect 13, is the polynucleotide of aspect 11 or 12, wherein the polynucleotide comprises a nucleic acid sequence with at least 80% sequence identity to SEQ ID NOs: 58-60.

Aspect 14, is the polynucleotide of any one of aspects 1 to 13, wherein the polynucleotide is operably linked to a regulatory sequence.

Aspect 15, is the polynucleotide of aspect 14, wherein the regulatory sequence comprises one or more of a promoter, a transcription initiation sequence, an internal ribosome entry site (IRES), an enhancer, an intron, an RNA interference target sequence, a Kozak sequence, splicing regulatory elements, and/or a polyadenylation signal.

Aspect 16, is the polynucleotide of any of aspects 1 to 15, wherein the polynucleotide is comprised in a vector and/or integrated into a genome.

Aspect 17, is the polynucleotide of aspect 16, wherein the vector is a plasmid, recombinant vector, and/or a viral vector.

Aspect 18, is the viral vector of aspect 17, wherein the viral vector comprises a retroviral, lentiviral, adenoviral, or adeno-associated viral packaging vector.

Aspect 19, is a polypeptide encoded by any one of aspects 1 to 18.

Aspect 20, is a polypeptide, comprising an extracellular stabilization domain comprising at least 80% sequence identity to SEQ ID NO: 13, an SRCR domain comprising at least 80% sequence identity to SEQ ID NO: 10, a hinge region, a transmembrane domain, and an intracellular signaling region.

Aspect 21, is the polypeptide of aspect 20, further comprising a CD6 d2-d3 linker region comprising at least 80% sequence identity to SEQ ID NO: 14.

Aspect 22, is the polypeptide of aspect 20 or 21, wherein the hinge region comprises a CD6 hinge region comprising at least 80% sequence identity to SEQ ID NO: 11.

Aspect 23, is the polypeptide of any one of aspects 20 to 22, wherein the transmembrane domain comprises a CD6 transmembrane region comprising at least 80% sequence identity to SEQ ID NO: 12.

Aspect 24, is the polypeptide of any one of aspects 20 to 23, wherein the intracellular signaling region comprises a CD6 intracellular signaling region comprising at least 80% sequence identity to SEQ ID NO: 2-8.

Aspect 25, is the polypeptide of any one of aspects 20 to 24, further comprising a signal peptide comprising at least 80% sequence identity to SEQ ID NO: 9.

Aspect 26, is the polypeptide of any one of aspects 20 to 25, further comprising a non-CD6 hinge region.

Aspect 27, is the polypeptide of aspect 26, wherein the non-CD6 hinge region comprises a CD4 and/or IgG1 hinge comprising at least 80% identity to SEQ ID NOs: 15 or 16.

Aspect 28, is the polypeptide of any one of aspects 20 to 27, wherein the extracellular stabilization domain comprises a sequence of at least 90% sequence identity to SEQ ID NO: 13.

Aspect 29, is the polypeptide of any one of aspects 20 to 28, wherein the SRCR domain comprises a CD6 SRCR domain comprising at least 90% identity to SEQ ID NOs: 10.

Aspect 30, is the polypeptide of any one of aspects 20 to 29, wherein the domains are operably joined in tandem.

Aspect 31, is the polypeptide of any one of aspects 20 to 30, wherein the polypeptide comprises an N- to C-terminus orientation in the following order: i) the extracellular stabilization domain, ii) the SRCR domain, iii) the hinge region, iv) transmembrane domain, and v) intracellular domain.

Aspect 32, is the polypeptide of any one of aspects 20 to 31, wherein the polypeptide comprises an amino acid sequence comprising at least 80% sequence identity to SEQ ID NOs: 22-24.

Aspect 33, is a method of making a transgenic avid cell expressing a recombinant protein, comprising transfecting, transforming, and/or transducing a cell in vitro and/or ex vivo with the polynucleotide and/or vector of any one of aspects 1 to 18.

Aspect 34, is the transgenic avid cell produced according to aspect 33.

Aspect 35, is the transgenic avid cell of aspect 34, wherein the cell is a mammalian cell.

Aspect 36, is the transgenic avid cell of aspect 34 or 35, wherein the cell is a mouse and/or human cell.

Aspect 37, is the transgenic avid cell of any one of aspects 34 to 36, wherein the cell is an immune cell.

Aspect 37, is the transgenic avid cell of aspect 37, wherein the immune cell is a T cell, natural killer T cell (NK T cell), macrophage, dendritic cell, and/or natural killer (NK) cell.

Aspect 39, is the transgenic avid cell of aspect 38, wherein the immune cell is a T cell or an NK cell.

Aspect 40, is the transgenic avid cell of any one of aspects 34 to 39, wherein the transgenic avid cell expresses one or more additional transgenes.

Aspect 41, is the transgenic avid cell of aspect 40, wherein the one or more additional transgenes comprise a sequence encoding an antigen recognition domain.

Aspect 42, is the transgenic avid cell of aspect 41, wherein the sequence encoding an antigen recognition domain encodes an antibody, antibody derivative, chimeric antigen receptor (CAR) and/or T-cell receptor (TCR).

Aspect 43, is the transgenic avid cell of aspect 42, wherein the transgenic avid cell expresses a CAR and/or TCR.

Aspect 44, is the CAR and/or TCR expressing transgenic avid cell of aspect 43, wherein the CAR and/or TCR targets a Human Epidermal Growth Factor Receptor 2 (Her2) and/or Cluster of Differentiation 19 (CD19) antigen.

Aspect 45, is the CAR and/or TCR expressing transgenic avid cell of aspect 44, wherein the CAR comprises a HER2.CD28ζ and/or a CD19.41BBζ CAR.

Aspect 46, is the transgenic avid cell of any one of aspects 34 to 45, wherein the transgenic cell exhibits greater avidity to a target cell relative to a control cell.

Aspect 47, is the transgenic avid cell of aspect 46, wherein the avidity of the transgenic cell to a target cell comprises a greater than two-fold increase relative to a control cell.

Aspect 48, is the transgenic avid cell of aspect 46, wherein the avidity of the transgenic cell to a target cell comprises a greater than four-fold increase relative to a control cell.

Aspect 49, is the transgenic avid cell of aspect 46, wherein the avidity of the transgenic cell to a target cell comprises a greater than six-fold increase relative to a control cell.

Aspect 50, is the transgenic avid cell of aspect 46, wherein the avidity of the transgenic cell to a target cell comprises a greater than eight-fold increase relative to a control cell.

Aspect 51, is the transgenic avid cell of aspect 46, wherein the avidity of the transgenic cell to a target cell comprises a greater than ten-fold increase relative to a control cell.

Aspect 52, is the transgenic avid cell of any one of aspects 46 to 51, wherein the control cell is a non-transgenic cell, and/or a non-avid transgenic cell.

Aspect 53, is the transgenic avid cell of any one of aspects 34 to 52, wherein the transgenic avid cell exhibits an increase in leukocyte adhesion glycoprotein 1 (LFA-1) recruitment to an immune synapse relative to a control cell.

Aspect 54, is the transgenic avid cell of any one of aspects 34 to 53, wherein the transgenic avid cell exhibits an increase in activated LFA-1 levels relative to a control cell.

Aspect 55, is the transgenic avid cell of any one of aspects 34 to 54, wherein the transgenic avid cell exhibits greater killing efficiency of a target cell relative to a control cell.

Aspect 56, is the transgenic avid cell of any one of aspects 53 to 55, wherein the control cell is a non-transgenic cell, and/or a non-avid transgenic cell.

Aspect 57, is the transgenic avid cell of any one of aspects 34 to 56, wherein the transgenic avid cell comprises one or more mutations in one or more endogenous genes.

Aspect 58, is a composition comprising the polynucleotide, polypeptide, and/or transgenic avid cell according to any one of the aspects 1 to 57.

Aspect 59, is the composition of aspect 58, further comprising a pharmaceutically acceptable carrier.

Aspect 60, is a method for treating an individual, comprising administering to the individual in need thereof, a polynucleotide, polypeptide, transgenic avid cell, and/or composition according to any one of aspects 1 to 59.

Aspect 61, is the method of aspect 60, wherein the individual has, is diagnosed with, and/or is suspected of having: cancer, an autoimmune disorder, a viral infection, neurological disorder, neurodegenerative disorder, and/or a bacterial infection.

Aspect 62, is the method of aspect 61, wherein the individual has, is diagnosed with, and/or is suspected of having cancer.

Aspect 63, is the method of aspect 62, wherein the cancer has a modest and/or low tumor associated antigen (TAA) expression level relative to other cancers expressing the same TAA.

Aspect 64, is the method of any one of aspects 61-63, wherein the cancer comprises glioblastoma and/or medulloblastoma.

Aspect 65, is the method of any one of aspects 61-64, wherein the cancer comprises glioblastoma multiform.

Aspect 66, is the method of any of aspects 60-65, wherein a patient has been diagnosed with a cancer having modest or low Her2 TAA expression relative to other cancers expressing Her2 TAA.

Aspect 67, is the method of aspect 60, wherein the transgenic avid cell comprises an autologous or allogenic cell.

Aspect 68, is a polynucleotide, comprising an extracellular stabilization domain encoding sequence comprising at least about 80% sequence identity to SEQ ID NO: 47 or 48, one or more extracellular domain encoding sequence, a transmembrane domain encoding sequence, and an intracellular signaling region encoding sequence, wherein the polynucleotide is operably linked to a non-endogenous promoter sequence.

Aspect 69, is the polynucleotide of aspect 68, wherein the one or more extracellular domain encoding sequence comprises a sequence comprising at least 80% sequence identity to CD6 extracellular domain 1 (d1) encoding sequence (SEQ ID NO: 53), a sequence comprising at least 80% sequence identity to CD6 extracellular domain 2 (d2) encoding sequence (SEQ ID NO: 55), and/or a sequence comprising at least 80% sequence identity to CD6 extracellular domain 3 (d3) encoding sequence (SEQ ID NOs: 39 or 40).

Aspect 70, is the polynucleotide of aspect 69, wherein the one or more extracellular domain encoding sequence comprises a sequence comprising at least 80% sequence identity to CD6 extracellular domain 1 (d1) encoding sequence (SEQ ID NO: 53), a sequence comprising at least 80% sequence identity to CD6 extracellular domain 2 (d2) encoding sequence (SEQ ID NO: 55), and does not comprise a d3 encoding sequence (SEQ ID NOs: 39 or 40).

Aspect 71, is the polynucleotide of aspect 69, wherein the d1, d2, and/or d3 encoding sequences comprise engineered mutations to alter the independent and/or combined functions of d1, d2, and/or d3, comprising at least about 80% sequence identity to SEQ ID NOs: 53, 55, 39, and/or 40.

Aspect 72, is the polynucleotide of any one of aspects 68 to 71, wherein the transmembrane domain encoding sequence comprises a CD6 transmembrane region encoding sequence comprising at least about 80% sequence identity to SEQ ID NOs: 43-46.

Aspect 73, is the polynucleotide of any one of aspects 68 to 72, wherein the intracellular signaling region encoding sequence comprises a CD6 intracellular signaling region encoding sequence comprising at least about 80% sequence identity to SEQ ID NOs: 28-36.

Aspect 74, is the polynucleotide of aspect 73, wherein the CD6 intracellular signaling region has been genetically engineered to modify the function of the CD6 intracellular signaling region relative to wild type full length CD6 protein as represented by SEQ ID NO: 1, comprising at least about 80% sequence identity to SEQ ID NOs: 28-36.

Aspect 75, is the polynucleotide of any one of aspects 68 to 74, wherein the encoding sequences are operably linked in tandem.

Aspect 76, is the polynucleotide of any one of aspects 68 to 75, comprising a 5′ to 3′ orientation of: i) the extracellular stabilization domain encoding sequence, ii) the one or more extracellular domain encoding sequence(s), iii) the transmembrane domain encoding sequence, and iv) the intracellular signaling region encoding sequence.

Aspect 77, is the polynucleotide of aspect 76, further comprising a nucleic acid sequence encoding a CD6 hinge region at least 80% identical to SEQ ID NOs: 41 or 42, interspersed between the one or more extracellular domain encoding sequences and the transmembrane domain encoding sequence.

Aspect 78, is the polynucleotide of aspect 76 or 77, further comprising a nucleic acid sequence encoding a CD6 ECS-D1 linker (SEQ ID NO: 52) interspersed between the extracellular stabilization domain and a first extracellular domain, and a nucleic acid sequence encoding a CD6 D1-D2 linker (SEQ ID NO: 54) comprised between two extracellular domain encoding sequences.

Aspect 79, is the polynucleotide of any one of aspects 68 to 78, further comprising a signal peptide encoding sequence, wherein optionally the signal peptide encoding sequence comprises a sequence at least about 80% identical to SEQ ID NOs: 37 or 38.

Aspect 80, is the polynucleotide of aspect of any one of aspects 76 to 79, wherein the polynucleotide comprises a nucleic acid sequence comprising at least 80% sequence identity to SEQ ID NO: 61.

Aspect 81, is the polynucleotide of any one of aspects 68 to 80, wherein the polynucleotide is operably linked to a regulatory sequence.

Aspect 82, is the polynucleotide of aspect 81, wherein the regulatory sequence comprises one or more of, a transcription initiation sequence, an internal ribosome entry site (IRES), an enhancer, an intron, an RNA interference target sequence, a Kozak sequence, splicing regulatory elements, and/or a polyadenylation signal.

Aspect 83, is the polynucleotide of any of aspects 68 to 82, wherein the polynucleotide is comprised in a vector and/or integrated into a genome.

Aspect 84, is the polynucleotide of aspect 83, wherein the vector is a plasmid, recombinant vector, and/or a viral vector.

Aspect 85, is the vector of aspect 84, wherein the vector is a viral vector, and the viral vector comprises a retroviral, lentiviral, adenoviral, or adeno-associated viral packaging vector.

Aspect 86, is a polypeptide encoded by the polynucleotide of any one of aspects 68 to 85.

Aspect 87, is a polypeptide, comprising an extracellular stabilization domain comprising at least about 80% sequence identity to SEQ ID NO: 13, one or more extracellular domains, a transmembrane domain, and an intracellular signaling region.

Aspect 88, is the polypeptide of aspect 87, wherein the one or more extracellular domains comprises an amino acid sequence comprising at least 80% sequence identity to CD6 extracellular domain 1 (d1) (SEQ ID NO: 18), a sequence comprising at least 80% sequence identity to CD6 extracellular domain 2 (d2) (SEQ ID NO: 20), and/or a sequence comprising at least 80% sequence identity to CD6 extracellular domain 3 (d3) (SEQ ID NO: 10).

Aspect 89, is the polypeptide of aspect 88, wherein the one or more extracellular domains comprises an amino acid sequence comprising an amino acid sequence comprising at least 80% sequence identity to CD6 extracellular domain 1 (d1) (SEQ ID NO: 18), a sequence comprising at least 80% sequence identity to CD6 extracellular domain 2 (d2) (SEQ ID NO: 20), and does not comprise a CD6 extracellular domain 3 (d3) amino acid sequence (SEQ ID NO: 10).

Aspect 90, is the polypeptide of aspect 88, wherein the d1, d2, and/or d3 comprising engineered mutations that modify the independent and/or combined endogenous functions of d1, d2, and/or d3, comprising at least about 80% sequence identity to SEQ ID NOs: 18, 20, and/or 10.

Aspect 91, is the polypeptide of any one of aspects 87 to 90, wherein the transmembrane domain comprises a CD6 transmembrane region comprising at least about 80% sequence identity to SEQ ID NO: 12.

Aspect 92, is the polypeptide of any one of aspects 87 to 91, wherein the intracellular signaling region comprises a CD6 intracellular signaling sequence comprising at least about 80% sequence identity to SEQ ID NOs: 2-8.

Aspect 93, is the polypeptide of aspect 92, wherein the CD6 intracellular signaling region comprises genetically engineered mutations modifying the function of the CD6 intracellular signaling region relative to endogenous functions, comprising at least about 80% sequence identity to SEQ ID NOs: 2-8.

Aspect 94, is the polypeptide of any one of aspects 87 to 93, wherein the domains and/or regions are operably linked in tandem.

Aspect 95, is the polypeptide of any one of aspects 87 to 94, comprising a N- to C-terminus orientation of: i) the extracellular stabilization domain, ii) the one or more extracellular domain(s), iii) the transmembrane domain, and vi) the intracellular signaling region.

Aspect 96, is the polypeptide of aspect 95, further comprising a CD6 hinge region sequence at least 80% identical to SEQ ID NO: 11, interspersed between the one or more extracellular domains and the transmembrane domain.

Aspect 97, is the polypeptide of aspect 95 or 96, further comprising a CD6 ECS-D1 linker sequence (SEQ ID NO: 17) interspersed between the extracellular stabilization domain and a first extracellular domain, and a CD6 D1-D2 linker sequence (SEQ ID NO: 19) comprised between two extracellular domains.

Aspect 98, is the polypeptide of any one of aspects 87 to 97, further comprising a signal peptide sequence, wherein optionally the signal peptide sequence comprises a sequence at least about 80% identical to SEQ ID NO: 9.

Aspect 99, is the polypeptide of any one of aspects of 87 to 98, wherein the polypeptide comprises an amino acid sequence comprising at least 80% sequence identity to SEQ ID NO: 25.

Aspect 100, is a method of making a transgenic avidity-weakened cell expressing a recombinant protein, comprising transfecting, transforming, and/or transducing a cell in vitro and/or ex vivo with the polynucleotide and/or vector of any one of aspects 68 to 85.

Aspect 101, is the transgenic avidity-weakened cell produced according to aspect 100.

Aspect 102, is the transgenic avidity-weakened cell of aspect 101, wherein the cell is a mammalian cell.

Aspect 103, is the transgenic avidity-weakened cell of aspect 101 or 102, wherein the cell is a mouse and/or human cell.

Aspect 104, is the transgenic avidity-weakened cell of any one of aspects 101 to 103, wherein the cell is an immune cell.

Aspect 105, is the transgenic avidity-weakened cell of aspect 104, wherein the immune cell is a T cell, natural killer T cell (NK T cell), macrophage, dendritic cell, and/or natural killer (NK) cell.

Aspect 106, is the transgenic avidity-weakened cell of aspect 105, wherein the immune cell is a T cell or an NK cell.

Aspect 107, is the transgenic avidity-weakened cell of any one of aspects 101 to 106, wherein the transgenic avidity-weakened cell expresses one or more additional transgenes.

Aspect 108, is the transgenic avidity-weakened cell of aspect 107, wherein the one or more additional transgenes comprise a sequence encoding an antigen recognition domain.

Aspect 109, is the transgenic avidity-weakened cell of aspect 108, wherein the sequence encoding an antigen recognition domain encodes an antibody, antibody derivative, CAR and/or TCR.

Aspect 110, is the transgenic avidity-weakened cell of aspect 109, wherein the transgenic avid-weakened cell expresses a CAR and/or TCR.

Aspect 111, is the CAR and/or TCR expressing transgenic avidity-weakened cell of aspect 110, wherein the CAR and/or TCR targets a Her2 and/or CD19 antigen.

Aspect 112, is the CAR and/or TCR expressing transgenic avidity-weakened cell of aspect 111, wherein the CAR comprises a HER2.CD28ζ and/or a CD19.41BBζ CAR.

Aspect 113, is the transgenic avidity-weakened cell of any one of aspects 101 to 112, wherein the transgenic avidity-weakened cell exhibits lower avidity to a target cell relative to a control cell, wherein the control cell is a transgenic non-avidity-weakened cell, non-transgenic cell, and/or an avid transgenic cell.

Aspect 114, is the transgenic avidity-weakened cell of aspect 113, wherein the avidity of the transgenic avidity-weakened cell to a target cell comprises a greater than two-fold decrease relative to a control cell, wherein the control cell is a transgenic non-avidity-weakened cell, non-transgenic cell, and/or an avid transgenic cell.

Aspect 115, is the transgenic avidity-weakened cell of any one of aspects 101 to 114, wherein the transgenic avid-weakened cell exhibits a decrease in LFA-1 recruitment to an immune synapse relative to a control cell, wherein the control cell is a transgenic non-avidity-weakened cell, non-transgenic cell, and/or an avid transgenic cell.

Aspect 116, is the transgenic avidity-weakened cell of any one of aspects 101 to 115, wherein the transgenic avid-weakened cell exhibits a decrease in activated LFA-1 levels relative to a control cell, wherein the control cell is a transgenic non-avidity-weakened cell, non-transgenic cell, and/or an avid transgenic cell.

Aspect 117, is the transgenic avidity-weakened cell of any one of aspects 101 to 116, wherein the transgenic avid-weakened cell exhibits greater killing efficiency of a target cell relative to a control cell, wherein the control cell is a transgenic non-avidity-weakened cell, non-transgenic cell, and/or an avid transgenic cell.

Aspect 118, is the transgenic avidity-weakened cell of any one of aspects 116 to 117, wherein the control cell is a transgenic non-avidity-weakened cell, non-transgenic cell, and/or an avid transgenic cell.

Aspect 119, is the transgenic avidity-weakened cell of any one of aspects 101 to 118, wherein the transgenic avidity-weakened cell comprises one or more mutations in one or more endogenous genes.

Aspect 120, is a composition comprising the polynucleotide, polypeptide, and/or transgenic avidity-weakened cell according to any one of the aspects 68 to 118.

Aspect 121, is the composition of aspect 120, further comprising a pharmaceutically acceptable carrier.

Aspect 122, is a method for treating an individual, comprising administering to the individual in need thereof, a polynucleotide, polypeptide, transgenic avidity-weakened cell, and/or composition according to any one of aspects 68 to 121.

Aspect 123, is the method of aspect 122, wherein the individual has, is diagnosed with, and/or is suspected of having: cancer, an autoimmune disorder, a viral infection, a neurological disorder, a neurodegenerative disorder, and/or a bacterial infection.

Aspect 124, is the method of aspect 123, wherein the individual has, is diagnosed with, and/or is suspected of having cancer.

Aspect 125, is the method of aspect 124, wherein the cancer has a medium and/or high TAA expression level relative to other cancers expressing the same TAA.

Aspect 126, is the method of any one of aspects 123 to 125, wherein the cancer comprises glioblastoma and/or medulloblastoma.

Aspect 127, is the method of any one of aspects 123 to 126, wherein the cancer comprises glioblastoma multiform.

Aspect 128, is the method of any one of aspects 122 to 127, wherein a patient has been diagnosed with a cancer having medium and/or high Her2 TAA expression relative to other cancers expressing Her2 TAA.

Aspect 129, is the method of any one of aspect 122 to 128, wherein the transgenic avidity-weakened cell comprises an autologous or allogenic cell.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins.

“Individual,” “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C, Modulation of immune synapse strength (avidity) to tune up or tune down immune cell activity. (1A) Is a graphic depicting a tumor cell in contact with an immune effector cell (e.g., T cell), and various binding and signaling actions that can occur & can potentially be modulated. (1B) Is a graphic depicting binding of CD6 extracellular (EC) domains with the target protein ALCAM (left panels), CD6 EC domain 3 (D3) is required for CD6-ALCAM interactions & subsequent signaling cascades/effects (e.g., LFA-1 inside-out activation, recruitment to the immune synapse, and/or actin remodeling), and loss of D3 abolishes CD6-ALCAM binding & potentially inhibits endogenous CD6-ALCAM interactions, e.g. in a dominant negative fashion. (1C) Confocal microscopy immunofluorescence images showing CD6 enriched at effector cell (e.g., “T” cells, e.g., T cells comprising Chimeric Antigen Receptor (CAR) targeting HER2 antigens) tumor cell (e.g. BT474, a HER2-amplified breast cancer cell line) Chimeric Antigen Receptor Immune Synapse (CARIS) sites, where colocalizations of actin, HER2 CARs (GFP tagged), CD6, and activated (high affinity) LFA-1 are apparent.

FIGS. 2A-2G, Engineered avidity switch polypeptides alter immune effector cell functions. (2A) Is a series of graphics representing exemplary engineered avidity switch polypeptides described herein (e.g., SEQ ID NOs: 21-25). Polypeptides may optionally comprise a marker molecule, such as a fluorophore (e.g., dsRed Mono). (2B) Are representative flow cytometry plots depicting CD6 (CD6=Full length transgenic CD6; D3=engineered transgenic avidity switch polypeptide) expression in exemplary transgenic immune cells described herein. (2C) Depicts the percentage decrease in transgenic T cells bound to target LN229 cells upon applying a force ramp of 1000 pico Newtons (pN). Exemplary immune effector cells expressed a HER2.CD28z CAR with or without the noted engineered avidity switch polypeptides (D3.0, D3.1, D3.2, and D3.3 respectively). (2D) Depicts exemplary flow cytometry results showing binding of soluble ALCAM by transgenic immune effector cells expressing the noted engineered avidity switch polypeptides; NT=non-transgenic control; 2ry only=secondary antibody only control. (2E) Depicts the distribution of force in pN needed to detach individual transgenic single cell T cells from their target LN229 cells. (2F) & (2G) Depicts exemplary representative flow cytometry results (2F) and associated quantification (2G) showing the pattern of LFA-1 activation in transgenic cells expressing construct D3.3 after activation with alpha-CD3 and alpha-CD28 antibodies; NT=non-transgenic (representative of 2 experiments); parametric T-test with Holm-Sidak adjustment for multiple comparisons, ns=non-significant, **=p-value<0.01.

FIGS. 3A-3C, Expression of a dominant negative CD6 polypeptide in immune effector cells. (3A) Depicts representative western blotting of endogenous full-length CD6 (CD6 FL) and dominant negative CD6 (CD6 ΔD3) in primary HER2 CAR T cells at baseline and 15 minutes post-exposure to BT474 cancer cells. (3B) Depicts the percentage of CD6 FL and CD6 ΔD3 of total CD6 at 0, 5, 16, and 60 minutes post T cell activation using anti-human CD3 and anti-human CD28 antibodies. (3C) Depicts the percentage of transduced T cells expressing a CAR (HER2.CD28z or CD19.41BBz) and CD6 ΔD3 (dsRed (CD6 tag)).

FIGS. 4A-4G, Modulated immune cell cytotoxicity achieved by modulation of immune cell avidity. (4A) Depicts the quantitative expression of HER2 as mean fluorescent intensity (MFI) from three representative cancer cell lines (DAOY, DAOY.2, and LN229). (4B) & (4C) Depicts an improvement in killing of DAOY cancer cells by HER2.CD28z CAR T-cells expressing d3.CD6 compared to HER2.CD28z only control cells or dominant negative (ΔD3.CD6) expressing cells (B) and the representative mean area under the curve (AUC) of each group (C) showing statistical differences; parametric T-test with Holm-Sidak adjustment for multiple comparisons, ***=p-value<0.0001. (4D) & (4E) Depicts an improvement in killing of DAOY.2 cancer cells by HER2.CD28z CAR T-cells expressing dominant negative CD6 (ΔD3.CD6) compared to HER2.CD28z only control cells (4D) and the representative mean area under the curve (AUC) of each group (4E) showing statistical differences; parametric T-test with Holm adjustment for multiple comparisons, ****=p-value<0.0001. (4F) Depicts the quantitative expression of CD19 by a variety of cancer cell lines (RAJI, JEKO, TOLEDO, NALM6) in comparison to a transduced DAOY cell line overexpressing CD19 (DAOY CD19). (4G) Depicts improved killing of DAOY CD19 cancer cells by CD19.41BBz CAR T-cells expressing dominant negative CD6 (ΔD3.CD6) compared to CD19.41BBz only control and CD19.41BBz d3 expressing cells.

### DETAILED DESCRIPTION

In some embodiments, aspects of the present disclosure are based, at least in part, on the generation of therapeutic immune effector cells (e.g., NK cells, T Cells, etc.) expressing one or more engineered avidity switch polypeptides, and optionally one or more molecules comprising antigen binding affinity (e.g., antigen receptors, antibodies, Chimeric Antigen Receptors, T Cell Receptors, etc.). In some embodiments, the present disclosure describes the discovery that Immune Synapses (IS), such as CAR mediated Immune Synapses (CARIS), can have their strength (e.g., avidity) modified through expression of one or more engineered avidity switch polypeptides.

In certain embodiments, immune synapse avidity modulates contact time between cells and directly affects the probability of antigen recognition. Consequently, in certain embodiments, immune effector cell activation is proportional to immune cell avidity at the immune synapse. In certain embodiments, in the case of immune effector cell therapies, such as CAR T cell therapy, immune cell avidity affects therapy outcomes. For example, in certain embodiments, low expression of a target antigen by a cancer cell can lead to antigen recognition escape and subsequent cancer relapse. Conversely, in certain embodiments, when antigen expression is high, immune effector cells can become exhausted, diminishing therapy efficacy. Ideally, as described herein in certain embodiments, immune effector cell activation would be proportional to the density of the target antigen to optimize antigen recognition, and/or immune cell activation and/or activity. In certain embodiments, the present disclosure allows immune effector cell activation modulation by altering immune cell avidity at the immune synapse using engineered avidity switch polypeptides.

In certain embodiments, an engineered avidity switch polypeptide can comprise Scavenger Receptor Cysteine-Rich (SRCR) domains (e.g., one or more CD6 SRCR domains) for increasing cellular avidity. Conversely, in certain embodiments, an engineered avidity switch polypeptide may be designed to, while not being limited by theory, act in a dominant negative fashion, through omission of an SRCR domain (e.g., may not comprise one or more CD6 SRCR domains). In some embodiments, such an engineered avidity switch polypeptide may dampen the avidity of an immune synapse. Accordingly, disclosed herein are polynucleotides, polypeptides, compositions, and/or immune cells comprising and/or encoding engineered avidity switch polypeptides, as well as methods for use of the same in treatment of disease (e.g., cancer, infectious diseases, autoimmune diseases, etc.). In various embodiments, the one or more engineered avidity switch polypeptides are heterologous to the cell in which it is comprised. In various embodiments, the one or more engineered avidity switch polypeptides are heterologous to the cell in which it is comprised and are not expressed from the genome of the cell. In various embodiments, the one or more avidity switch polypeptides are expressed from a synthetic vector generated by the hand of man.

### I. Proteins

As used herein, a “protein” or “polypeptide” refers to a molecule comprising at least five amino acid residues. As used herein, a “peptide” refers to a molecule comprising at least three amino acid residues. As used herein, the term “wild-type” refers to the endogenous version of a molecule that occurs naturally in an organism. In some aspects, wild-type versions of a protein or polypeptide are employed, however, in aspects of the engineered avidity switch protein of the disclosure, a modified protein or polypeptide is employed to act as an avidity modifying agent, such as reducing or increasing the avidity of an immune effector cell to a target cell at an immune synapse. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an engineered avidity switch polypeptide, an antibody or fragment thereof, a chimeric antigen receptor, etc.). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization (e.g., a marker, such as dsRed mono), for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides or proteins of the disclosure may comprise, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids, or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750, or more contiguous amino acids, or any range derivable therein, of SEQ ID NOs: 1-26.

In some aspects, a protein or polypeptide may comprise, consist essentially of, or consist of amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750 (or any derivable range therein) of SEQ ID NOs: 1-26.

In some aspects, a protein or polypeptide may comprise, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750 (or any derivable range therein) contiguous amino acids of SEQ ID NOs: 1-26.

In some aspects, the polypeptide or protein may comprise, consist essentially of, or consist of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750 (or any derivable range therein) contiguous amino acids of SEQ ID NOs: 1-26 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs: 1-26.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750 of any of SEQ ID NOs: 1-26 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, or 750 (or any derivable range therein) contiguous amino acids of any of SEQ ID NOs: 1-26.

In some embodiments, provided herein are nucleotide as well as the protein, polypeptide, and peptide sequences for various genes. In certain embodiments, wild type copies of certain proteins, polypeptides, and/or nucleotides described herein have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Engineered Avidity Switches

In certain embodiments, aspects of the present disclosure include an engineered molecule, that can alter the avidity of an immune cell to a target. In certain embodiments, the engineered molecule is an engineered polypeptide. In certain embodiments, the engineered polypeptide is an engineered avidity switch polypeptide. In certain embodiments, an engineered polypeptide comprises domains of a CD6 polypeptide (CD6 is also known as “T-cell differentiation antigen CD6,” “TP120,” or “T12”). CD6 is present on T cells and is known to play a role in fine-tuning and maintaining T Cell Receptor (TCR) signal strength. In certain embodiments, an engineered polypeptide of the disclosure may comprise human CD6 polypeptide(s) or non-human CD6 polypeptide(s) (e.g., murine CD6 polypeptides). In certain embodiments, an engineered polypeptide of the disclosure may comprise polypeptides associated with CD6 alternatively spliced isoforms, some of which lack various portions of the cytoplasmic tail of the protein. In certain embodiments, an engineered polypeptide of the disclosure comprises CD6 polypeptides including the full length intracellular sequence & associated protein domains, e.g., comprising CD6A signaling domains (referred to herein as the “canonical” CD6 isoform). In some embodiments, an engineered polypeptide comprises CD6 polypeptides including comprising a shorter intracellular sequence and associated protein domains, e.g., including but not limited to isoforms B, C, D, E, and F (CD6B, CD6C, CD6D, CD6E, and CD6F respectively, referred to herein as “non-canonical” CD6 isoforms). Example sequences of engineered polypeptides and constitutive parts thereof (e.g., polypeptide sequences and/or domains such as interaction and/or signaling domains, linker sequences, hinge sequences, etc.) are provided in the table below. In certain embodiments, an engineered polypeptide can comprise, consist of, and/or consist essentially of any one or more of SEQ ID NOs: 1-26.

In certain embodiments, aspects of the present disclosure include an engineered avidity switch, nucleic acids encoding an engineered avidity switch, and cells comprising an engineered avidity switch. In certain embodiments, an engineered avidity switch comprises domains of a CD6 polypeptide (CD6 is also known as “T-cell differentiation antigen CD6,” “TP120,” or “T12”). An engineered avidity switch protein of the disclosure may comprise human CD6 polypeptide(s) or non-human CD6 polypeptide(s) (e.g., murine CD6 polypeptides). In certain embodiments, an engineered avidity switch may comprise polypeptides associated with CD6 alternatively spliced isoforms, some of which lack various portions of the cytoplasmic tail of the protein. In certain embodiments, an engineered avidity switch comprises CD6 polypeptides including the full length intracellular sequence & associated protein domains, e.g., comprising CD6A signaling domains (referred to herein as the “canonical” CD6 isoform). In some embodiments, an engineered avidity switch comprises CD6 polypeptides including comprising a shorter intracellular sequence and associated protein domains, e.g., including but not limited to isoforms B, C, D, E, and F (CD6B, CD6C, CD6D, CD6E, and CD6F respectively, referred to herein as “non-canonical” CD6 isoforms). Example sequences of engineered avidity switch proteins and constitutive parts thereof (e.g., polypeptide sequences and/or domains such as interaction and/or signaling domains, linker sequences, hinge sequences, etc.) are provided in the table below. In certain embodiments, an engineered avidity switch can comprise, consist of, and/or consist essentially of any one or more of SEQ ID NOs: 1-26.

In certain embodiments, an engineered avidity switch polypeptide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.1 (SEQ ID NO: 22).

In certain embodiments, an engineered avidity switch polypeptide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.2 (SEQ ID NO: 23).

In certain embodiments, an engineered avidity switch polypeptide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.3 (SEQ ID NO: 24).

In certain embodiments, an engineered avidity switch polypeptide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 ΔD3 (SEQ ID NO: 25).

### B. Extracellular Stabilization Domain

In certain embodiments, aspects of the present disclosure include an extracellular stabilization domain (ESD), nucleic acids encoding an ESD, and cells comprising an ESD. In certain embodiments, the ESD comprises the intrinsically disordered region of a protein. Intrinsically disordered regions in proteins are polypeptide segments that generally lack enough hydrophobic amino acids to mediate a defined three-dimensional, or tertiary, structure. Intrinsically disordered regions are known to potentially modulate protein functions and/or activity, such as exposing motifs to facilitate target interactions, allowing a wider range of interactive partners, modulating interaction strength, serving as a scaffold, regulating enzyme activity, serving as a substrate for post-translation modifications, regulating protein half-life, and/or modulating different conformational states. Consequently, intrinsically disordered regions can regulate binding of cell-surface proteins to their targets and downstream signaling. In certain embodiments, the extracellular stabilization domain comprises the intrinsically disordered region of CD6. In certain embodiments, the extracellular stabilization domain facilitates binding of the engineered avidity switch to its target. In certain embodiments, the extracellular stabilization domain comprises an intrinsically disordered region known in the art, for example but not limited to the intrinsically disordered region of CD5 and/or CD6.

In certain embodiments, an extracellular stabilization domain comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 EC stabilization (ECS) domain (SEQ ID NO: 13).

### C. Linkers and Hinges

In certain embodiments, aspects of the present disclosure include linkers and/or hinges, nucleic acids encoding linkers and/or hinges, and cells comprising linkers and/or hinges. In certain embodiments, the linkers and/or hinges comprise polypeptide sequences from a protein known in the art. Linkers and/or hinges are sequences of amino acids that provide structure and/or flexibility to a protein. In certain embodiments, the linkers and/or hinges may improve protein stability, extracellular domain presentation, and/or ligand binding. In certain embodiments, the linkers and/or hinger comprise a region of the CD6 protein. In certain embodiments, the linkers and/or hinger comprise a region of the CD4 protein. In certain embodiments, the linkers and/or hingers comprise a region of the Immunoglobulin G 1 (IgG1) protein. In certain embodiments, the linkers and/or hinges comprise suitable linkers and/or hinges known in the art, for example but not limited to regions of the IgG2, IgG3, IgG4, IgM, IgA, IgD, IgE, IgG2a, IgG2b, IgG3, IgA1, and/or IgA2. In certain embodiments, the linkers and/or hinges are derived from suitable organisms known in the art, for example but not limited to human, mouse, guinea pig, rabbit, goat, bovine, swine, and/or ovine.

In certain embodiments, a linker comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D2-D3 linker (SEQ ID NO: 14).

In certain embodiments, a linker comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 ECS-D1 linker (SEQ ID NO: 17).

In certain embodiments, a linker comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D1-D2 linker (SEQ ID NO: 19).

In certain embodiments, a hinge comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 hinge (SEQ ID NO: 11)

In certain embodiments, a hinge comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD4, d3d4 hinge (SEQ ID NO: 15).

In certain embodiments, a hinge comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to IgG1 hinge (SEQ ID NO: 16).

### D. Scavenger Receptor Cysteine-Rich Domains

In certain embodiments, aspects of the present disclosure include scavenger receptor cysteine rich (SRCR) domains, nucleic acids encoding SRCR domains, and cells comprising SRCR domains. SRCR domains are protein domains, typically found in secreted and cell surface proteins, such as extracellular receptors and cell-adhesion molecules, that can mediate binding of the protein to a target. For example, the CD6 protein encodes three SRCR domains (termed d1, d2, and d3 from N-terminus to C-terminus) that play different roles in CD6 binding to its targets, such as ALCAM (also known as CD166). In particular, the d3 domain is required for efficient binding between CD6 and ALCAM, an interaction that can strengthen and stabilize the immune synapse, as well as effect intracellular signaling and actin remodeling through the interaction of the CD6 intracellular domain to Lymphocyte function-associated antigen 1 (LFA-1). In certain embodiments, compositions comprise one or more SRCR domains. In certain embodiments, the SRCR domains comprise the d1, d2, and/or d3 SRCR domains of CD6. In certain embodiments, the SRCR domains comprise the SRCR domains of other suitable proteins known in the art, for example but not limited to CD5 and/or CD6.

In certain embodiments, an SRCR domain comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 domain D1 (SEQ ID NO: 18).

In certain embodiments, an SRCR domain comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 domain D2 (SEQ ID NO: 20).

In certain embodiments, an SRCR domain comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3 domain (SEQ ID NO: 10).

TABLE 1
Polypeptide Sequences

| | | SEQ |
|---|---|---|
| Name | Sequence | ID NO |
| Human CD6 | MWLFFGITGLLTAALSGHPSPAPPDQLNTSSAESELWEPGERLPV | 1 |
| isoform A | RLTNGSSSCSGTVEVRLEASWEPACGALWDSRAAEAVCRALGCGG | |
| (CD6A) | AEAASQLAPPTPELPPPPAAGNTSVAANATLAGAPALLCSGAEWR | |
| | LCEVVEHACRSDGRRARVTCAENRALRLVDGGGACAGRVEMLEHG | |
| | EWGSVCDDTWDLEDAHVVCRQLGCGWAVQALPGLHFTPGRGPIHR | |
| | DQVNCSGAEAYLWDCPGLPGQHYCGHKEDAGAVCSEHQSWRLTGG | |
| | ADRCEGQVEVHFRGVWNTVCDSEWYPSEAKVLCQSLGCGTAVERP | |
| | KGLPHSLSGRMYYSCNGEELTLSNCSWRFNNSNLCSQSLAARVLC | |
| | SASRSLHNLSTPEVPASVQTVTIESSVTVKIENKESRELMLLIPS | |
| | IVLGILLLGSLIFIAFILLRIKGKYALPVMVNHQHLPTTIPAGSN | |
| | SYQPVPITIPKEVFMLPIQVQAPPPEDSDSGSDSDYEHYDFSAQP | |
| | PVALTTFYNSQRHRVTDEEVQQSRFQMPPLEEGLEELHASHIPTA | |
| | NPGHCITDPPSLGPQYHPRSNSESSTSSGEDYCNSPKSKLPPWNP | |
| | QVFSSERSSFLEQPPNLELAGTQPAFSAGPPADDSSSTSSGEWYQ | |
| | NFQPPPQPPSEEQFGCPGSPSPQPDSTDNDDYDDISAA | |
| CD6A, IC | LRIKGKYALPVMVNHQHLPTTIPAGSNSYQPVPITIPKEVEMLPI | 2 |
| signaling | QVQAPPPEDSDSGSDSDYEHYDFSAQPPVALTTFYNSQRHRVTDE | |
| domains | EVQQSRFQMPPLEEGLEELHASHIPTANPGHCITDPPSLGPQYHP | |
| | RSNSESSTSSGEDYCNSPKSKLPPWNPQVESSERSSFLEQPPNLE | |
| | LAGTQPAFSAGPPADDSSSTSSGEWYQNFQPPPQPPSEEQFGCPG | |
| | SPSPQPDSTDNDDYDDISAA | |
| CD6B, IC | LRIKGKYVFMLPIQVQAPPPEDSDSGSDSDYEHYDFSAQPPVALT | 3 |
| signaling | TFYNSQRHRVTDEEVQQSRFQMPPLEEGLEELHASHIPTANPGHC | |
| domains | ITDPPSLGPQYHPRSNSESSTSSGEDYCNSPKSKLPPWNPQVFSS | |
| | ERSSFLEQPPNLELAGTQPAFSAGPPADDSSSTSSGEWYQNFQPP | |
| | PQPPSEEQFGCPGSPSPQPDSTDNDDYDDISAA | |
| CD6C, IC | LRIKGKYDSQRHRVTDEEVQQSRFQMPPLEEGLEELHASHIPTAN | 4 |
| signaling | PGHCITDPPSLGPQYHPRSNSESSTSSGEDYCNSPKSKLPPWNPQ | |
| domains | VFSSERSSFLEQPPNLELAGTQPAFSAGPPADDSSSTSSGEWYQN | |
| | FQPPPQPPSEEQFGCPGSPSPQPDSTDNDDYDDISAA | |
| CD6D, IC | LRIKGKYVFMLPIQVQAPPPEDSDSGSDSDYEHYDFSAQPPVALT | 5 |
| signaling | TFYNSQRHRVTDEEVQQSRFQMPPLEEGLEELHASHIPTANPGHC | |
| domains | ITDPPSLGPQYHPRSNSESSTSSGEDYCNSPKSKLPPWNPQVFSS | |
| | ERSSFLEQPPNLELAGTQPAFSGSPSPQPDSTDNDDYDDISAA | |
| CD6E, IC | LRIKGKYALPVMVNHQHLPTTIPAGSNSYQPVPITIPKEDSQRHR | 6 |
| signaling | VTDEEVQQSRFQMPPLEEGLEELHASHIPTANPGHCITDPPSLGP | |
| domains | QYHPRSNSESSTSSGEDYCNSPKSKLPPWNPQVESSERSSFLEQP | |
| | PNLELAGTQPAFSGSPSPQPDSTDNDDYDDISAA | |
| CD6F, IC | LRIKGKYALPVMVNHQHLPTTIPAGSNSYQPVPITIPKESQRHRV | 7 |
| signaling | TDEEVQQSRFQMPPLEEGLEELHASHIPTANPGHCITDPPSLGPQ | |
| domains | YHPRSNSESSTSSGEDYCNSPKSKLPPWNPQVFSSERSSFLEQPP | |
| | NLELAGTQPAFSAGPPADDSSSTSSGEWYQNFQPPPQPPSEEQFG | |
| | CPGSPSPQPDSTDNDDYDDISAA | |
| CD6 AD3 IC | LRIKGKYALPVMVNHQHLPTTIPAGSNSYQPVPITIPKEVFMLPI | 8 |
| signaling | QVQAPPPEDSDSGSDSDYEHYDFSAQPPVALTTFYNSQRHRVTDE | |
| domains | EVQQSRFQMPPLEEGLEELHASHIPTANPGHCITDPPSLGPQYHP | |
| | RSNSESSTSSGEDYCNSPKSKLPPWNPQVFSSERSSFLEQPPNLE | |
| | LASTQPAFSAGPPADDSSSTSSGEWYQNFQPPPQPPSEEQFGCPG | |
| | SPSPQPDSTDNDDYDDISAA | |
| CD6 Signal | MWLFFGITGLLTAALSG | 9 |
| Peptide | | |
| CD6 D3 | WRLTGGADRCEGQVEVHFRGVWNTVCDSEWYPSEAKVLCQSLGCG | 10 |
| domain | TAVERPKGLPHSLSGRMYYSCNGEELTLSNCSWRFNNSNLCSQSL | |
| | AARVLCS | |
| CD6 hinge | ASRSLHNLSTPEVPASVQTVTIESSVTVKIENKESRELMLL | 11 |
| domain | | |
| CD6 TM | IPSIVLGILLLGSLIFIAFIL | 12 |
| domain | | |
| CD6 EC | HPSPAPPDQLNTSSAESELWEPG | 13 |
| stabilization | | |
| (ECS) domain | | |
| CD6 D2-D3 | CSEHQS | 14 |
| Linker | | |
| CD4.d3d4 | FQKASSIVYKKEGEQVEFSFPLAFTVEKLTGSGELWWQAERASSS | 15 |
| hinge | KSWITFDLKNKEVSVKRVTQDPKLQMGKKLPLHLTLPQALPQYAG | |
| | SGNLTLALEAKTGKLHQEVNLVVMRATQLQKNLTCEVWGPTSPKL | |
| | MLSLKLENKEAKVSKREKAVWVLNPEAGMWQCLLSD | |
| IgG1 hinge | EDPAEPKSPDKTHTCPPCPAPELLGGPSVFLFPPKPKDTLMISRT | 16 |
| | PEVTCVVVDVSHEDPEVKFNWYVDGVEVHNAKTKPREEQYNSTYR | |
| | VVSVLTVLHQDWLNGKEYKCKVSNKALPAPIEKTISKAKGQPREP | |
| | QVYTLPPSRDELTKNQVSLTCLVKGFYPSDIAVEWESNGQPENNY | |
| | KTTPPVLDSDGSFFLYSKLTVDKSRWQQGNVFSCSVMHEALHNHY | |
| | TQKSLSLSPGKKDPK | |
| CD6 ECS-D1 | ERLP | 17 |
| Linker | | |
| CD6 domain | VRLTNGSSSCSGTVEVRLEASWEPACGALWDSRAAEAVCRALGCG | 18 |
| D1 | GAEAASQLAPPTPELPPPPAAGNTSVAANATLAGAPALLCSGAEW | |
| | RLCEVVEHACRSDGRRARVTCA | |
| CD6 D1-D2 | ENRA | 19 |
| Linker | | |
| CD6 domain | LRLVDGGGACAGRVEMLEHGEWGSVCDDTWDLEDAHVVCRQLGCG | 20 |
| D2 | WAVQALPGLHFMPGRGPIHRDQVNCSGAEAYLWDCPGLPGQHYCG | |
| | HKEDAGVVCS | |
| CD6 D3.0 | MWLFFGITGLLTAALSGWRLTGGADRCEGQVEVHFRGVWNTVCDS | 21 |
| | EWYPSEAKVLCQSLGCGTAVERPKGLPHSLSGRMYYSCNGEELTL | |
| | SNCSWRFNNSNLCSQSLAARVLCSASRSLHNLSTPEVPASVQTVT | |
| | IESSVTVKIENKESRELMLLIPSIVLGILLLGSLIFIAFILLRIK | |
| | GKYALPVMVNHQHLPTTIPAGSNSYQPVPITIPKEVFMLPIQVQA | |
| | PPPEDSDSGSDSDYEHYDESAQPPVALTTFYNSQRHRVTDEEVQQ | |
| | SRFQMPPLEEGLEELHASHIPTANPGHCITDPPSLGPQYHPRSNS | |
| | ESSTSSGEDYCNSPKSKLPPWNPQVFSSERSSFLEQPPNLELAGT | |
| | QPAFSAGPPADDSSSTSSGEWYQNFQPPPQPPSEEQFGCPGSPSP | |
| | QPDSTDNDDYDDISAA | |
| CD6 D3.1 | MWLFFGITGLLTAALSGHPSPAPPDQLNTSSAESELWEPGCSEHQ | 22 |
| | SWRLTGGADRCEGQVEVHFRGVWNTVCDSEWYPSEAKVLCQSLGC | |
| | GTAVERPKGLPHSLSGRMYYSCNGEELTLSNCSWRFNNSNLCSQS | |
| | LAARVLCSASRSLHNLSTPEVPASVQTVTIESSVTVKIENKESRE | |
| | LMLLIPSIVLGILLLGSLIFIAFILLRIKGKYALPVMVNHQHLPT | |
| | TIPAGSNSYQPVPITIPKEVFMLPIQVQAPPPEDSDSGSDSDYEH | |
| | YDFSAQPPVALTTFYNSQRHRVTDEEVQQSRFQMPPLEEGLEELH | |
| | ASHIPTANPGHCITDPPSLGPQYHPRSNSESSTSSGEDYCNSPKS | |
| | KLPPWNPQVFSSERSSFLEQPPNLELAGTQPAFSAGPPADDSSST | |
| | SSGEWYQNFQPPPQPPSEEQFGCPGSPSPQPDSTDNDDYDDISAA | |
| CD6 D3.2 | MWLFFGITGLLTAALSGHPSPAPPDQLNTSSAESELWEPGCSEHQ | 23 |
| | SWRLTGGADRCEGQVEVHFRGVWNTVCDSEWYPSEAKVLCQSLGC | |
| | GTAVERPKGLPHSLSGRMYYSCNGEELTLSNCSWRENNSNLCSQS | |
| | LAARVLCSASRSLHNLSTPEVPASVQTVTIESSVTVKIENKESRE | |
| | LMLLFQKASSIVYKKEGEQVEFSFPLAFTVEKLTGSGELWWQAER | |
| | ASSSKSWITFDLKNKEVSVKRVTQDPKLQMGKKLPLHLTLPQALP | |
| | QYAGSGNLTLALEAKTGKLHQEVNLVVMRATQLQKNLTCEVWGPT | |
| | SPKLMLSLKLENKEAKVSKREKAVWVLNPEAGMWQCLLSDIPSIV | |
| | LGILLLGSLIFIAFILLRIKGKYALPVMVNHQHLPTTIPAGSNSY | |
| | QPVPITIPKEVEMLPIQVQAPPPEDSDSGSDSDYEHYDFSAQPPV | |
| | ALTTFYNSQRHRVTDEEVQQSRFQMPPLEEGLEELHASHIPTANP | |
| | GHCITDPPSLGPQYHPRSNSESSTSSGEDYCNSPKSKLPPWNPQV | |
| | FSSERSSFLEQPPNLELAGTQPAFSAGPPADDSSSTSSGEWYQNF | |
| | QPPPQPPSEEQFGCPGSPSPQPDSTDNDDYDDISAA | |
| CD6 D3.3 | MWLFFGITGLLTAALSGHPSPAPPDQLNTSSAESELWEPGCSEHQ | 24 |
| | SWRLTGGADRCEGQVEVHFRGVWNTVCDSEWYPSEAKVLCQSLGC | |
| | GTAVERPKGLPHSLSGRMYYSCNGEELTLSNCSWRFNNSNLCSQS | |
| | LAARVLCSASRSLHNLSTPEVPASVQTVTIESSVTVKIENKESRE | |
| | LMLLEDPAEPKSPDKTHTCPPCPAPELLGGPSVFLFPPKPKDTLM | |
| | ISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGKKDPKIPSIVLGILLLGSLIFIAFILLRIKG | |
| | KYALPVMVNHQHLPTTIPAGSNSYQPVPITIPKEVEMLPIQVQAP | |
| | PPEDSDSGSDSDYEHYDFSAQPPVALTTFYNSQRHRVTDEEVQQS | |
| | RFQMPPLEEGLEELHASHIPTANPGHCITDPPSLGPQYHPRSNSE | |
| | SSTSSGEDYCNSPKSKLPPWNPQVESSERSSFLEQPPNLELAGTQ | |
| | PAFSAGPPADDSSSTSSGEWYQNFQPPPQPPSEEQFGCPGSPSPQ | |
| | PDSTDNDDYDDISAA | |
| CD6 ΔD3 | MWLFFGITGLLTAALSGHPSPAPPDQLNTSSAESELWEPGERLPV | 25 |
| | RLTNGSSSCSGTVEVRLEASWEPACGALWDSRAAEAVCRALGCGG | |
| | AEAASQLAPPTPELPPPPAAGNTSVAANATLAGAPALLCSGAEWR | |
| | LCEVVEHACRSDGRRARVTCAENRALRLVDGGGACAGRVEMLEHG | |
| | EWGSVCDDTWDLEDAHVVCRQLGCGWAVQALPGLHEMPGRGPIHR | |
| | DQVNCSGAEAYLWDCPGLPGQHYCGHKEDAGVVCSASRSLHNLST | |
| | PEVPASVQTVTIESSVTVKIENKESRELMLLIPSIVLGILLLGSL | |
| | IFIAFILLRIKGKYALPVMVNHQHLPTTIPAGSNSYQPVPITIPK | |
| | EVFMLPIQVQAPPPEDSDSGSDSDYEHYDESAQPPVALTTFYNSQ | |
| | RHRVTDEEVQQSRFQMPPLEEGLEELHASHIPTANPGHCITDPPS | |
| | LGPQYHPRSNSESSTSSGEDYCNSPKSKLPPWNPQVESSERSSFL | |
| | EQPPNLELASTQPAFSAGPPADDSSSTSSGEWYQNFQPPPQPPSE | |
| | EQFGCPGSPSPQPDSTDNDDYDDISAA | |
| dsRed | MDNTEDVIKEFMQFKVRMEGSVNGHYFEIEGEGEGKPYEGTQTAK | 26 |
| Monomer | LQVTKGGPLPFAWDILSPQFQYGSKAYVKHPADIPDYMKLSFPEG | |
| | FTWERSMNFEDGGVVEVQQDSSLQDGTFIYKVKFKGVNFPADGPV | |
| | MQKKTAGWEPSTEKLYPQDGVLKGEISHALKLKDGGHYTCDFKTV | |
| | YKAKKPVQLPGNHYVDSKLDITNHNEDYTVVEQYEHAEARHSGSQ | |

*IC = Intracellular; TM = Transmembrane; EC = Extracellular;

### II. Nucleic Acids

Aspects of the disclosure include nucleic acids. In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, chimeric antigen receptor, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

As will be understood by those in the art, the term “nucleic acid” or “polynucleotide” encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, an engineered avidity switch can comprise sequences encoded by any one or more of SEQ ID NOs: 27-62.

In certain embodiments, an engineered avidity switch encoding polynucleotide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.1 (SEQ ID NO: 58).

In certain embodiments, an engineered avidity switch encoding polynucleotide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.2 (SEQ ID NO: 59).

In certain embodiments, an engineered avidity switch encoding polynucleotide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 D3.3 (SEQ ID NO: 60).

In certain embodiments, an engineered avidity switch encoding polynucleotide comprises, consists essentially of, or consists of a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to CD6 ΔD3 (SEQ ID NO: 61).

In certain embodiments, an engineered avidity switch encoding polynucleotide comprising a sequence at least or exactly 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any range derivable therein, identical to an extracellular stabilization domain (SEQ ID NOs: 47 or 48) has significantly improved stability, signaling capacity, membrane localization, and/or receptor interaction rates when compared to an engineered avidity switch encoding polynucleotide that does not comprise an extracellular stabilization domain.

TABLE 2
Polynucleotide Sequences

| | | SEQ |
|---|---|---|
| Name | Sequence | ID NO |
| Human CD6 | ATGTGGCTCTTCTTCGGGATCACTGGATTGCTGACGGCAGCCCTC | 27 |
| isoform A | TCAGGTCATCCATCTCCAGCCCCACCTGACCAGCTCAACACCAGC | |
| (CD6A) | AGTGCAGAGAGTGAGCTCTGGGAGCCAGGGGAGCGGCTTCCGGTC | |
| | CGTCTGACAAACGGGAGCAGCAGCTGCAGCGGGACGGTGGAGGTG | |
| | CGGCTCGAGGCGTCCTGGGAGCCCGCGTGCGGGGCGCTCTGGGAC | |
| | AGCCGCGCCGCCGAGGCCGTGTGCCGAGCACTGGGCTGCGGCGGG | |
| | GCGGAGGCCGCCTCTCAGCTCGCCCCGCCGACCCCTGAGCTGCCG | |
| | CCCCCGCCTGCAGCCGGGAACACCAGCGTAGCAGCTAATGCCACT | |
| | CTGGCCGGGGCGCCCGCCCTCCTGTGCAGCGGCGCCGAGTGGCGG | |
| | CTCTGCGAGGTGGTGGAGCACGCGTGCCGCAGCGACGGGAGGCGG | |
| | GCCCGTGTCACCTGTGCAGAGAACCGCGCGCTGCGCCTGGTGGAC | |
| | GGTGGCGGCGCCTGCGCCGGCCGCGTGGAGATGCTGGAGCATGGC | |
| | GAGTGGGGATCAGTGTGCGATGACACTTGGGACCTGGAGGACGCC | |
| | CACGTGGTGTGCAGGCAACTGGGCTGCGGCTGGGCAGTCCAGGCC | |
| | CTGCCCGGCTTGCACTTCACGCCCGGCCGCGGGCCTATCCACCGG | |
| | GACCAGGTGAACTGCTCGGGGGCCGAAGCTTACCTGTGGGACTGC | |
| | CCGGGGCTGCCAGGACAGCACTACTGCGGCCACAAAGAGGACGCG | |
| | GGCGCGGTGTGCTCAGAGCACCAGTCCTGGCGCCTGACAGGGGGC | |
| | GCTGACCGCTGCGAGGGGCAGGTGGAGGTACACTTCCGAGGGGTC | |
| | TGGAACACAGTGTGTGACAGTGAGTGGTACCCATCGGAGGCCAAG | |
| | GTGCTCTGCCAGTCCTTGGGCTGTGGAACTGCGGTTGAGAGGCCC | |
| | AAGGGGCTGCCCCACTCCTTGTCCGGCAGGATGTACTACTCATGC | |
| | AATGGGGAGGAGCTCACCCTCTCCAACTGCTCCTGGCGGTTCAAC | |
| | AACTCCAACCTCTGCAGCCAGTCGCTGGCAGCCAGGGTCCTCTGC | |
| | TCAGCTTCCCGGAGTTTGCACAATCTGTCCACTCCCGAAGTCCCT | |
| | GCAAGTGTTCAGACAGTCACTATAGAATCTTCTGTGACAGTGAAA | |
| | ATAGAGAACAAGGAATCTCGGGAGCTAATGCTCCTCATCCCCTCC | |
| | ATCGTTCTGGGAATTCTCCTCCTTGGCTCCCTCATCTTCATAGCC | |
| | TTCATCCTCTTGAGAATTAAAGGAAAATATGCCCTCCCCGTAATG | |
| | GTGAACCACCAGCACCTACCCACCACCATCCCGGCAGGGAGCAAT | |
| | AGCTATCAACCGGTCCCCATCACCATCCCCAAAGAAGTTTTCATG | |
| | CTGCCCATCCAGGTCCAGGCCCCGCCCCCTGAGGACTCAGACTCT | |
| | GGCTCGGACTCAGACTATGAGCACTATGACTTCAGCGCCCAGCCT | |
| | CCTGTGGCCCTGACCACCTTCTACAATTCCCAGCGGCATCGGGTC | |
| | ACAGATGAGGAGGTCCAGCAAAGCAGGTTCCAGATGCCACCCTTG | |
| | GAGGAAGGACTTGAAGAGTTGCATGCCTCCCACATCCCAACTGCC | |
| | AACCCTGGACACTGCATTACAGACCCGCCATCCCTGGGCCCTCAG | |
| | TATCACCCGAGGAGCAACAGTGAGTCGAGCACCTCTTCAGGGGAG | |
| | GATTACTGCAATAGTCCCAAAAGCAAGCTGCCTCCATGGAACCCC | |
| | CAGGTGTTTTCTTCAGAGAGGAGTTCCTTCCTGGAGCAGCCCCCA | |
| | AACTTGGAGCTGGCCGGCACCCAGCCAGCCTTTTCAGCAGGGCCC | |
| | CCGGCTGATGACAGCTCCAGCACCTCATCCGGGGAGTGGTACCAG | |
| | AACTTCCAGCCACCACCCCAGCCCCCTTCGGAGGAGCAGTTTGGC | |
| | TGTCCAGGGTCCCCCAGCCCTCAGCCTGACTCCACCGACAACGAT | |
| | GACTACGATGACATCAGCGCAGCCTAG | |
| CD6A, IC | CTGAGGATCAAGGGCAAATACGCCCTGCCTGTGATGGTCAACCAC | 28 |
| signaling | CAGCATCTGCCCACAACAATCCCAGCCGGCAGCAATAGCTACCAG | |
| domains | CCTGTGCCTATCACAATCCCCAAAGAGGTGTTCATGCTGCCCATC | |
| | CAGGTTCAGGCCCCACCTCCTGAGGATAGCGATAGCGGCAGCGAT | |
| | TCCGACTACGAGCACTACGATTTCAGCGCCCAGCCTCCTGTGGCT | |
| | CTGACCACCTTCTACAACAGCCAGAGACACAGAGTGACCGACGAG | |
| | GAAGTGCAGCAGAGCAGATTCCAGATGCCTCCTCTGGAAGAAGGC | |
| | CTGGAAGAACTGCACGCCTCTCACATCCCTACAGCCAATCCTGGC | |
| | CACTGCATCACCGATCCTCCATCTCTGGGACCCCAGTATCACCCC | |
| | AGAAGCAATAGCGAGTCTAGCACCAGCAGCGGCGAGGACTACTGC | |
| | AACAGCCCTAAGTCTAAGCTGCCACCTTGGAACCCTCAGGTGTTC | |
| | AGCAGCGAGAGAAGCAGCTTCCTGGAACAGCCTCCTAACCTGGAA | |
| | CTGGCCGGAACACAGCCTGCCTTTTCTGCTGGACCTCCTGCCGAT | |
| | GATAGCAGCAGCACATCCAGCGGCGAATGGTATCAGAACTTCCAG | |
| | CCACCTCCTCAGCCTCCAAGCGAGGAACAGTTTGGCTGTCCTGGC | |
| | TCTCCATCACCTCAGCCTGACAGCACCGACAACGACGACTACGAT | |
| | GACATCAGCGCCGCT | |
| CD6B, IC | TTGAGAATTAAAGGAAAATATGTTTTCATGCTGCCCATCCAGGTC | 29 |
| signaling | CAGGCCCCGCCCCCTGAGGACTCAGACTCTGGCTCGGACTCAGAC | |
| domains | TATGAGCACTATGACTTCAGCGCCCAGCCTCCTGTGGCCCTGACC | |
| | ACCTTCTACAATTCCCAGCGGCATCGGGTCACAGATGAGGAGGTC | |
| | CAGCAAAGCAGGTTCCAGATGCCACCCTTGGAGGAAGGACTTGAA | |
| | GAGTTGCATGCCTCCCACATCCCAACTGCCAACCCTGGACACTGC | |
| | ATTACAGACCCGCCATCCCTGGGCCCTCAGTATCACCCGAGGAGC | |
| | AACAGTGAGTCGAGCACCTCTTCAGGGGAGGATTACTGCAATAGT | |
| | CCCAAAAGCAAGCTGCCTCCATGGAACCCCCAGGTGTTTTCTTCA | |
| | GAGAGGAGTTCCTTCCTGGAGCAGCCCCCAAACTTGGAGCTGGCC | |
| | GGCACCCAGCCAGCCTTTTCAGGGCCCCCGGCTGATGACAGCTCC | |
| | AGCACCTCATCCGGGGAGTGGTACCAGAACTTCCAGCCACCACCC | |
| | CAGCCCCCTTCGGAGGAGCAGTTTGGCTGTCCAGGGTCCCCCAGC | |
| | CCTCAGCCTGACTCCACCGACAACGATGACTACGATGACATCAGC | |
| | GCAGCCTAG | |
| CD6C, IC | TTGAGAATTAAAGGAAAATATGATTCCCAGCGGCATCGGGTCACA | 30 |
| signaling | GATGAGGAGGTCCAGCAAAGCAGGTTCCAGATGCCACCCTTGGAG | |
| domains | GAAGGACTTGAAGAGTTGCATGCCTCCCACATCCCAACTGCCAAC | |
| | CCTGGACACTGCATTACAGACCCGCCATCCCTGGGCCCTCAGTAT | |
| | CACCCGAGGAGCAACAGTGAGTCGAGCACCTCTTCGGGGGAGGAT | |
| | TACTGCAATAGTCCCAAAAGCAAGCCGCCTCCATGGAACCCCCAG | |
| | GTGTTTTCTTCAGAGAGGAGTTCCTTCCTGGAGCAGCCCCCAAAC | |
| | TTGGAGCTGGCCGGCACCCAGCCAGCCTTTTCAGCAGGGCCCCCG | |
| | GCTGATGACAGCTCCAGCACCTCATCCGGGGAGTGGTACCAGAAC | |
| | TTCCAGCCACCACCCCAGCCCCCTTCGGAGGAGCAGTTTGGCTGT | |
| | CCAGGGTCCCCCAGCCCTCAGCCTGACTCCACCGACAACGATGAC | |
| | TACGATGACATAGGCGCAGCCTAG | |
| CD6D, IC | TTGAGAATTAAAGGAAAATATGTTTTCATGCTGCCCATCCAGGTC | 31 |
| signaling | CAGGCCCCGCCCCCTGAGGACTCAGACTCTGGCTCGGACTCAGAC | |
| domains | TATGAGCACTATGACTTCAGCGCCCAGCCTCCTGTGGCCCTGACC | |
| | ACCTTCTACAATTCCCAGCGGCATCGGGTCACAGATGAGGAGGTC | |
| | CAGCAAAGCAGGTTCCAGATGCCACCCTTGGAGGAAGGACTTGAA | |
| | GAGTTGCATGCCTCCCACATCCCAACTGCCAACCCTGGACACTGC | |
| | ATTACAGACCCGCCATCCCTGGGCCCTCAGTATCACCCGAGGAGC | |
| | AACAGTGAGTCGAGCACCTCTTCAGGGGAGGATTACTGCAATAGT | |
| | CCCAAAAGCAAGCTGCCTCCATGGAACCCCCAGGTGTTTTCTTCA | |
| | GAGAGGAGTTCCTTCCTGGAGCAGCCCCCAAACTTGGAGCTGGCC | |
| | GGCACCCAGCCAGCCTTTTCAGGGTCCCCCAGCCCTCAGCCTGAC | |
| | TCCACCGACAACGATGACTACGATGACATCAGCGCAGCCTAG | |
| CD6E, IC | TTGAGAATTAAAGGAAAATATGCCCTCCCCGTAATGGTGAACCAC | 32 |
| signaling | CAGCACCTACCCACCACCATCCCGGCAGGGAGCAATAGCTATCAA | |
| domains | CCGGTCCCCATCACCATCCCCAAAGAAGATTCCCAGCGGCATCGG | |
| | GTCACAGATGAGGAGGTCCAGCAAAGCAGGTTCCAGATGCCACCC | |
| | TTGGAGGAAGGACTTGAAGAGTTGCATGCCTCCCACATCCCAACT | |
| | GCCAACCCTGGACACTGCATTACAGACCCGCCATCCCTGGGCCCT | |
| | CAGTATCACCCGAGGAGCAACAGTGAGTCGAGCACCTCTTCAGGG | |
| | GAGGATTACTGCAATAGTCCCAAAAGCAAGCTGCCTCCATGGAAC | |
| | CCCCAGGTGTTTTCTTCAGAGAGGAGTTCCTTCCTGGAGCAGCCC | |
| | CCAAACTTGGAGCTGGCCGGCACCCAGCCAGCCTTTTCAGGGTCC | |
| | CCCAGCCCTCAGCCTGACTCCACCGACAACGATGACTACGATGAC | |
| | ATCAGCGCAGCCTAG | |
| CD6F, IC | CTGAGGATCAAGGGCAAATACGCCCTGCCTGTGATGGTCAACCAC | 33 |
| signaling | CAGCATCTGCCCACAACAATCCCTGCCGGAAGCAACAGCTACCAG | |
| domains | CCTGTGCCTATCACAATCCCCAAAGAGTCCCAGAGACACCGCGTG | |
| | ACCGATGAGGAAGTGCAGCAGAGCAGATTCCAGATGCCTCCTCTG | |
| | GAAGAAGGCCTGGAAGAACTGCACGCCTCTCACATCCCTACAGCC | |
| | AATCCTGGCCACTGCATCACCGATCCTCCATCTCTGGGCCCTCAG | |
| | TATCACCCCAGAAGCAATAGCGAGTCTAGCACCAGCAGCGGCGAG | |
| | GACTACTGCAACAGCCCTAAGTCTAAGCTGCCTCCATGGAATCCC | |
| | CAGGTGTTCAGCAGCGAGAGATCCAGCTTTCTGGAACAGCCTCCT | |
| | AACCTGGAACTGGCCTCTACACAGCCTGCCTTTTCTGCCGGACCT | |
| | CCAGCCGATGATAGCAGCTCTACAAGCTCTGGCGAATGGTATCAG | |
| | AACTTCCAGCCTCCTCCACAGCCTCCAAGCGAGGAACAGTTTGGA | |
| | TGTCCTGGCAGCCCATCTCCTCAGCCTGACAGCACCGACAACGAC | |
| | GACTACGATGATATCAGCGCTGCC | |
| CD6A, IC | CTGAGGATCAAGGGCAAATACGCCCTGCCTGTGATGGTCAACCAC | 34 |
| signaling | CAGCATCTGCCCACAACAATCCCAGCCGGCAGCAATAGCTACCAG | |
| domains v2 | CCTGTGCCTATCACAATCCCCAAAGAAGTGTTCATGCTGCCCATC | |
| | CAGGTTCAGGCCCCACCTCCTGAGGATAGCGATAGCGGCAGCGAT | |
| | TCCGACTACGAGCACTACGATTTCAGCGCCCAGCCTCCTGTGGCT | |
| | CTGACCACCTTCTACAACAGCCAGAGACACAGAGTGACCGACGAG | |
| | GAAGTGCAGCAGAGCAGATTCCAGATGCCTCCTCTGGAAGAAGGC | |
| | CTGGAAGAACTGCACGCCTCTCACATCCCTACAGCCAATCCTGGC | |
| | CACTGCATCACCGATCCTCCATCTCTGGGACCCCAGTATCACCCC | |
| | AGAAGCAATAGCGAGTCTAGCACCAGCAGCGGCGAGGACTACTGC | |
| | AACAGCCCTAAGTCTAAGCTGCCACCTTGGAACCCTCAGGTGTTC | |
| | AGCAGCGAGAGAAGCAGCTTTCTCGAGCAGCCTCCTAACCTGGAA | |
| | CTGGCCGGAACACAGCCTGCCTTTTCTGCTGGACCTCCTGCCGAT | |
| | GATAGCAGCAGCACATCCAGCGGCGAATGGTATCAGAACTTCCAG | |
| | CCACCTCCTCAGCCTCCAAGCGAGGAACAGTTTGGCTGTCCTGGC | |
| | TCTCCATCACCTCAGCCTGACAGCACCGACAACGACGACTACGAT | |
| | GACATCAGCGCCGCT | |
| CD6A, IC | CTGAGGATCAAGGGCAAATACGCCCTGCCAGTGATGGTCAACCAC | 35 |
| signaling | CAGCATCTGCCTACAACAATCCCAGCCGGCAGCAACTCTTACCAG | |
| domains v3 | CCTGTGCCAATCACAATCCCCAAAGAGGTGTTCATGCTGCCCATC | |
| | CAGGTTCAGGCCCCACCTCCTGAGGATAGCGATAGCGGCAGCGAT | |
| | TCCGACTACGAGCACTACGACTTTAGCGCCCAGCCTCCTGTGGCT | |
| | CTGACCACCTTCTACAACAGCCAGAGACACAGAGTGACCGACGAG | |
| | GAAGTGCAGCAGAGCAGATTCCAGATGCCTCCTCTGGAAGAAGGC | |
| | CTGGAAGAACTGCACGCCTCTCACATCCCTACCGCCAATCCAGGC | |
| | CACTGCATCACCGATCCTCCATCTCTGGGACCCCAGTATCACCCC | |
| | AGAAGCAATAGCGAGAGCAGCACCTCTAGCGGCGAGGACTACTGC | |
| | AACAGCCCTAAGTCTAAGCTGCCACCTTGGAATCCCCAGGTGTTC | |
| | AGCAGCGAGAGAAGCAGCTTTCTGGAACAGCCTCCTAACCTGGAA | |
| | CTGGCCGGAACACAGCCTGCCTTTTCTGCTGGACCTCCTGCCGAT | |
| | GATAGCAGCTCTACAAGCAGCGGCGAATGGTATCAGAACTTCCAG | |
| | CCACCTCCTCAGCCACCTAGCGAAGAACAGTTTGGCTGTCCTGGC | |
| | AGCCCATCTCCACAGCCAGACAGCACCGACAACGACGACTACGAT | |
| | GACATCAGCGCCGCT | |
| CD6A, IC | TTGAGAATTAAAGGAAAATATGCCCTCCCCGTAATGGTGAACCAC | 36 |
| signaling | CAGCACCTACCCACCACCATCCCGGCAGGGAGCAATAGCTATCAA | |
| domains v4 | CCGGTCCCCATCACCATCCCCAAAGAAGTTTTCATGCTGCCCATC | |
| | CAGGTCCAGGCCCCGCCCCCTGAGGACTCAGACTCTGGCTCGGAC | |
| | TCAGACTATGAGCACTATGACTTCAGCGCCCAGCCTCCTGTGGCC | |
| | CTGACCACCTTCTACAATTCCCAGCGGCATCGGGTCACAGATGAG | |
| | GAGGTCCAGCAAAGCAGGTTCCAGATGCCACCCTTGGAGGAAGGA | |
| | CTTGAAGAGTTGCATGCCTCCCACATCCCAACTGCCAACCCTGGA | |
| | CACTGCATTACAGACCCGCCATCCCTGGGCCCTCAGTATCACCCG | |
| | AGGAGCAACAGTGAGTCGAGCACCTCTTCGGGGGAGGATTACTGC | |
| | AATAGTCCCAAAAGCAAGCTGCCTCCATGGAACCCCCAGGTGTTT | |
| | TCTTCAGAGAGGAGTTCCTTCCTGGAGCAGCCCCCAAACTTGGAG | |
| | CTGGCCAGCACCCAGCCAGCCTTTTCAGCAGGGCCCCCGGCTGAT | |
| | GACAGCTCCAGCACCTCATCCGGGGAGTGGTACCAGAACTTCCAG | |
| | CCACCACCCCAGCCCCCTTCGGAGGAGCAGTTTGGCTGTCCAGGG | |
| | TCCCCCAGCCCTCAGCCTGACTCCACCGACAACGATGACTACGAT | |
| | GACATCAGCGCAGCC | |
| CD6 Signal | ATGTGGCTGTTCTTTGGCATCACCGGACTGCTGACAGCCGCTCTG | 37 |
| Peptide | TCTGGA | |
| CD6 Signal | ATGTGGCTCTTCTTCGGGATCACTGGATTGCTGACGGCAGCCCTC | 38 |
| Peptide v2 | TCAGGT | |
| CD6 D3 | TGGAGACTGACCGGCGGAGCCGATAGATGTGAAGGACAGGTCGAG | 39 |
| domain | GTGCACTTCAGAGGCGTGTGGAATACCGTGTGCGACAGCGAGTGG | |
| | TATCCTAGCGAGGCCAAGGTGCTGTGTCAGTCTCTTGGCTGTGGA | |
| | ACCGCCGTGGAAAGACCTAAGGGACTGCCTCACAGCCTGAGCGGC | |
| | AGAATGTACTACAGCTGCAACGGCGAGGAACTGACCCTGAGCAAC | |
| | TGCAGCTGGCGGTTCAACAACAGCAACCTGTGTAGCCAGAGCCTG | |
| | GCCGCCAGAGTGCTGTGTTCT | |
| CD6 D3 | TGGAGACTGACCGGCGGAGCCGATAGATGTGAAGGACAGGTCGAG | 40 |
| domain v2 | GTGCACTTCAGAGGCGTGTGGAACACCGTGTGCGACAGCGAGTGG | |
| | TATCCTTCTGAGGCCAAGGTGCTGTGTCAGTCTCTCGGATGTGGC | |
| | ACCGCCGTGGAAAGACCTAAGGGACTGCCTCACAGCCTGAGCGGC | |
| | AGAATGTACTACAGCTGCAACGGCGAGGAACTGACCCTGAGCAAC | |
| | TGCAGCTGGCGGTTCAACAACAGCAACCTGTGTAGCCAGAGCCTG | |
| | GCCGCCAGAGTGCTGTGTTCT | |
| CD6 hinge | GCCAGCAGAAGCCTGCACAACCTGAGCACACCTGAAGTGCCTGCC | 41 |
| | AGCGTGCAGACAGTGACAATCGAGAGCAGCGTGACCGTGAAGATC | |
| | GAGAACAAAGAAAGCCGCGAGCTGATGCTGCTG | |
| CD6 hinge v2 | GCTTCCCGGAGTTTGCACAATCTGTCCACTCCCGAAGTCCCTGCA | 42 |
| | AGTGTTCAGACAGTCACTATAGAATCTTCTGTGACAGTGAAAATA | |
| | GAGAACAAGGAATCTCGGGAGCTAATGCTCCTC | |
| CD6 TM | ATCCCTTCTATCGTGCTGGGCATCCTGCTGCTGGGCTCCCTGATC | 43 |
| domain | TTTATCGCCTTTATCCTG | |
| CD6 TM | ATCCCTAGCATCGTGCTGGGAATCCTGCTGCTGGGCAGCCTGATC | 44 |
| domain v2 | TTTATCGCCTTCATCCTG | |
| CD6 TM | ATCCCTAGCATCGTGCTGGGCATCCTGCTGCTGGGCTCCCTGATC | 45 |
| domain v3 | TTTATCGCCTTTATCCTG | |
| CD6 TM | ATCCCCTCCATCGTTCTGGGAATTCTCCTCCTTGGCTCCCTCATC | 46 |
| domain v4 | TTCATAGCCTTCATCCTC | |
| CD6 EC | CATCCTTCTCCAGCTCCACCTGACCAGCTGAATACCAGCTCTGCC | 47 |
| stabilization | GAGTCTGAGCTGTGGGAGCCTGGA | |
| (ECS) domain | | |
| CD6 EC | CATCCATCTCCAGCCCCACCTGACCAGCTCAACACCAGCAGTGCA | 48 |
| stabilization | GAGAGTGAGCTCTGGGAGCCAGGG | |
| (ECS) domain | | |
| v2 | | |
| CD6 D2-D3 | TGTTCTGAGCACCAGTCT | 49 |
| Linker | | |
| CD4.d3d4 | TTCCAGAAAGCCAGCTCCATCGTGTACAAGAAAGAGGGCGAGCAG | 50 |
| hinge | GTCGAGTTCAGCTTCCCTCTGGCCTTCACCGTGGAAAAGCTGACA | |
| | GGATCTGGCGAACTGTGGTGGCAGGCCGAAAGAGCCTCTAGCAGC | |
| | AAGAGCTGGATCACCTTCGACCTGAAAAACAAAGAGGTGTCCGTC | |
| | AAGCGCGTGACACAGGACCCTAAGCTGCAGATGGGAAAGAAGCTG | |
| | CCCCTGCACCTGACACTGCCTCAGGCTTTGCCTCAGTATGCCGGC | |
| | TCTGGAAATCTGACACTGGCCCTGGAAGCCAAGACCGGCAAACTG | |
| | CATCAAGAAGTGAACCTGGTCGTCATGCGGGCCACACAGCTGCAG | |
| | AAGAATCTGACCTGCGAAGTGTGGGGCCCTACAAGCCCTAAACTG | |
| | ATGCTGAGCCTGAAGCTGGAAAACAAAGAAGCCAAGGTTTCCAAG | |
| | CGCGAGAAGGCCGTGTGGGTGTTGAATCCTGAAGCCGGCATGTGG | |
| | CAGTGCCTGCTGAGCGAT | |
| IgG1 hinge | GAAGATCCTGCCGAGCCTAAGAGCCCTGACAAGACCCACACATGC | 51 |
| | CCTCCATGTCCTGCTCCAGAACTGCTCGGCGGACCCTCCGTTTTC | |
| | CTGTTTCCACCTAAGCCTAAGGACACCCTGATGATCAGCAGAACC | |
| | CCTGAAGTGACCTGCGTGGTGGTGGATGTGTCTCACGAGGACCCC | |
| | GAAGTGAAGTTCAATTGGTACGTGGACGGCGTGGAAGTGCACAAC | |
| | GCCAAGACCAAGCCTAGAGAGGAACAGTACAACAGCACCTACAGA | |
| | GTGGTGTCCGTGCTGACCGTGCTGCACCAGGATTGGCTGAACGGC | |
| | AAAGAGTACAAGTGCAAGGTGTCCAACAAGGCCCTGCCTGCTCCT | |
| | ATCGAAAAGACCATCAGCAAGGCCAAGGGCCAGCCAAGAGAACCC | |
| | CAGGTTTACACCCTGCCTCCAAGCAGAGATGAGCTGACCAAGAAC | |
| | CAGGTGTCCCTGACCTGCCTGGTCAAGGGCTTCTACCCTAGCGAC | |
| | ATTGCCGTGGAATGGGAGAGCAATGGCCAGCCTGAGAACAACTAC | |
| | AAGACCACACCTCCTGTGCTGGACAGCGACGGCTCATTCTTCCTG | |
| | TACAGCAAGCTGACAGTGGACAAGAGCAGATGGCAGCAGGGCAAC | |
| | GTGTTCAGCTGTTCTGTGATGCACGAGGCCCTGCACAATCACTAC | |
| | ACCCAGAAGTCCCTGTCTCTGAGCCCAGGCAAGAAGGACCCTAAG | |
| CD6 ECS-D1 | GAGCGGCTTCCG | 52 |
| Linker | | |
| CD6 D1 | GTCCGTCTGACAAACGGGAGCAGCAGCTGCAGCGGGACGGTGGAG | 53 |
| domain | GTGCGGCTCGAGGCGTCCTGGGAGCCCGCGTGCGGGGCGCTCTGG | |
| | GACAGCCGCGCCGCCGAGGCCGTGTGCCGAGCACTGGGCTGCGGC | |
| | GGGGCGGAGGCCGCCTCTCAGCTCGCCCCGCCGACCCCTGAGCTG | |
| | CCGCCCCCGCCTGCAGCCGGGAACACCAGCGTAGCAGCTAATGCC | |
| | ACTCTGGCCGGGGCGCCCGCCCTCCTGTGCAGCGGCGCCGAGTGG | |
| | CGGCTCTGCGAGGTGGTGGAGCACGCGTGCCGCAGCGACGGGAGG | |
| | CGGGCCCGTGTCACCTGTGCA | |
| CD6 D1-D2 | GAGAACCGCGCG | 54 |
| Linker | | |
| CD6 D2 | CTGCGCCTGGTGGACGGTGGCGGCGCCTGCGCCGGCCGCGTGGAG | 55 |
| domain | ATGCTGGAGCATGGCGAGTGGGGATCAGTGTGCGATGACACTTGG | |
| | GACCTGGAGGACGCCCACGTGGTGTGCAGGCAACTGGGCTGCGGC | |
| | TGGGCAGTCCAGGCCCTGCCCGGCTTGCACTTCATGCCCGGCCGC | |
| | GGGCCTATCCACCGGGACCAGGTGAACTGCTCGGGGGCCGAAGCT | |
| | TACCTGTGGGACTGCCCGGGGCTGCCAGGACAGCACTACTGCGGC | |
| | CACAAAGAGGACGCGGGCGTGGTGTGCTCA | |
| CD6 5′ UTR | CCTCTAGACTGCC | 56 |
| CD6 D3.0 | ATGTGGCTGTTCTTCGGCATCACCGGACTGCTGACAGCCGCTTTA | 57 |
| | TCTGGATGGCGACTGACCGGCGGAGCCGATAGATGTGAAGGACAG | |
| | GTGGAAGTGCACTTTCGGGGCGTGTGGAACACCGTGTGCGACAGC | |
| | GAGTGGTATCCTAGCGAGGCCAAGGTGCTGTGTCAGAGCCTGGGA | |
| | TGTGGCACCGCCGTGGAAAGACCTAAGGGACTGCCTCACAGCCTG | |
| | AGCGGCAGAATGTACTACAGCTGCAACGGCGAGGAACTGACCCTG | |
| | AGCAATTGCAGCTGGCGGTTCAACAACAGCAACCTGTGCAGCCAG | |
| | TCTCTGGCCGCCAGAGTGCTGTGTAGCGCCAGCAGAAGCCTGCAC | |
| | AATCTGAGCACACCTGAGGTGCCAGCCAGCGTGCAGACAGTGACA | |
| | ATCGAGAGCAGCGTGACCGTGAAGATCGAGAACAAAGAAAGCCGC | |
| | GAGCTGATGCTGCTGATCCCCAGCATCGTGCTGGGAATCCTGCTG | |
| | CTGGGCAGCCTGATCTTTATCGCCTTCATCCTGCTGCGGATCAAG | |
| | GGCAAATACGCCCTGCCCGTGATGGTCAACCACCAGCATCTGCCT | |
| | ACAACCATCCCTGCCGGCAGCAACAGCTACCAGCCTGTGCCCATC | |
| | ACCATCCCCAAAGAGGTGTTCATGCTGCCCATCCAGGTGCAGGCC | |
| | CCTCCACCTGAGGATAGCGATAGCGGCAGCGACAGCGACTACGAG | |
| | CACTACGATTTTTCCGCCCAGCCTCCTGTGGCCCTGACCACCTTC | |
| | TACAACAGCCAGAGACACAGAGTGACCGACGAGGAAGTGCAGCAG | |
| | AGCAGATTCCAGATGCCCCCTCTGGAAGAGGGCCTGGAAGAACTG | |
| | CATGCCAGCCACATCCCTACCGCCAATCCTGGCCACTGCATCACC | |
| | GATCCTCCAAGCCTGGGACCCCAGTACCACCCCAGAAGCAATAGC | |
| | GAGAGCAGCACCAGCAGCGGCGAGGACTACTGCAACAGCCCTAAG | |
| | AGCAAGCTGCCCCCCTGGAACCCTCAGGTGTTCAGCAGCGAGAGA | |
| | AGCAGCTTCCTGGAACAGCCCCCCAATCTGGAACTGGCCGGAACA | |
| | CAGCCTGCCTTTTCTGCCGGACCTCCTGCCGATGATAGCAGCAGC | |
| | ACAAGCTCCGGGGAGTGGTATCAGAACTTCCAGCCTCCTCCACAG | |
| | CCCCCTAGCGAGGAACAGTTTGGCTGTCCTGGAAGCCCCAGCCCT | |
| | CAGCCCGATAGCACCGACAACGACGACTACGACGATATCAGCGCC | |
| | GCT | |
| CD6 D3.1 | ATGTGGCTGTTCTTTGGCATCACCGGACTGCTGACAGCCGCTCTG | 58 |
| | TCTGGACATCCTTCTCCAGCTCCACCTGACCAGCTGAATACCAGC | |
| | TCTGCCGAGTCTGAGCTGTGGGAGCCTGGATGTTCTGAGCACCAG | |
| | TCTTGGAGACTGACCGGCGGAGCCGATAGATGTGAAGGACAGGTC | |
| | GAGGTGCACTTCAGAGGCGTGTGGAATACCGTGTGCGACAGCGAG | |
| | TGGTATCCTAGCGAGGCCAAGGTGCTGTGTCAGTCTCTTGGCTGT | |
| | GGAACCGCCGTGGAAAGACCTAAGGGACTGCCTCACAGCCTGAGC | |
| | GGCAGAATGTACTACAGCTGCAACGGCGAGGAACTGACCCTGAGC | |
| | AACTGCAGCTGGCGGTTCAACAACAGCAACCTGTGTAGCCAGAGC | |
| | CTGGCCGCCAGAGTGCTGTGTTCTGCCAGCAGAAGCCTGCACAAC | |
| | CTGAGCACACCTGAAGTGCCTGCCAGCGTGCAGACAGTGACAATC | |
| | GAGAGCAGCGTGACCGTGAAGATCGAGAACAAAGAAAGCCGCGAG | |
| | CTGATGCTGCTGATCCCTTCTATCGTGCTGGGCATCCTGCTGCTG | |
| | GGCTCCCTGATCTTTATCGCCTTTATCCTGCTGAGGATCAAGGGC | |
| | AAATACGCCCTGCCTGTGATGGTCAACCACCAGCATCTGCCCACA | |
| | ACAATCCCAGCCGGCAGCAATAGCTACCAGCCTGTGCCTATCACA | |
| | ATCCCCAAAGAGGTGTTCATGCTGCCCATCCAGGTTCAGGCCCCA | |
| | CCTCCTGAGGATAGCGATAGCGGCAGCGATTCCGACTACGAGCAC | |
| | TACGATTTCAGCGCCCAGCCTCCTGTGGCTCTGACCACCTTCTAC | |
| | AACAGCCAGAGACACAGAGTGACCGACGAGGAAGTGCAGCAGAGC | |
| | AGATTCCAGATGCCTCCTCTGGAAGAAGGCCTGGAAGAACTGCAC | |
| | GCCTCTCACATCCCTACAGCCAATCCTGGCCACTGCATCACCGAT | |
| | CCTCCATCTCTGGGACCCCAGTATCACCCCAGAAGCAATAGCGAG | |
| | TCTAGCACCAGCAGCGGCGAGGACTACTGCAACAGCCCTAAGTCT | |
| | AAGCTGCCACCTTGGAACCCTCAGGTGTTCAGCAGCGAGAGAAGC | |
| | AGCTTCCTGGAACAGCCTCCTAACCTGGAACTGGCCGGAACACAG | |
| | CCTGCCTTTTCTGCTGGACCTCCTGCCGATGATAGCAGCAGCACA | |
| | TCCAGCGGCGAATGGTATCAGAACTTCCAGCCACCTCCTCAGCCT | |
| | CCAAGCGAGGAACAGTTTGGCTGTCCTGGCTCTCCATCACCTCAG | |
| | CCTGACAGCACCGACAACGACGACTACGATGACATCAGCGCCGCT | |
| CD6 D3.2 | ATGTGGCTGTTCTTTGGCATCACCGGACTGCTGACAGCCGCTCTG | 59 |
| | TCTGGACATCCTTCTCCAGCTCCACCTGACCAGCTGAATACCAGC | |
| | TCTGCCGAGTCTGAGCTGTGGGAGCCTGGATGTTCTGAGCACCAG | |
| | TCTTGGAGACTGACCGGCGGAGCCGATAGATGTGAAGGACAGGTC | |
| | GAGGTGCACTTCAGAGGCGTGTGGAATACCGTGTGCGACAGCGAG | |
| | TGGTATCCTAGCGAGGCCAAGGTGCTGTGTCAGTCTCTTGGCTGT | |
| | GGAACCGCCGTGGAAAGACCTAAGGGACTGCCTCACAGCCTGAGC | |
| | GGCAGAATGTACTACAGCTGCAACGGCGAGGAACTGACCCTGAGC | |
| | AACTGCAGCTGGCGGTTCAACAACAGCAACCTGTGTAGCCAGAGC | |
| | CTGGCCGCCAGAGTGCTGTGTTCTGCCAGCAGAAGCCTGCACAAC | |
| | CTGAGCACACCTGAAGTGCCTGCCAGCGTGCAGACAGTGACAATC | |
| | GAGAGCAGCGTGACCGTGAAGATCGAGAACAAAGAAAGCCGCGAG | |
| | CTGATGCTGCTGTTCCAGAAAGCCAGCTCCATCGTGTACAAGAAA | |
| | GAGGGCGAGCAGGTCGAGTTCAGCTTCCCTCTGGCCTTCACCGTG | |
| | GAAAAGCTGACAGGATCTGGCGAACTGTGGTGGCAGGCCGAAAGA | |
| | GCCTCTAGCAGCAAGAGCTGGATCACCTTCGACCTGAAAAACAAA | |
| | GAGGTGTCCGTCAAGCGCGTGACACAGGACCCTAAGCTGCAGATG | |
| | GGAAAGAAGCTGCCCCTGCACCTGACACTGCCTCAGGCTTTGCCT | |
| | CAGTATGCCGGCTCTGGAAATCTGACACTGGCCCTGGAAGCCAAG | |
| | ACCGGCAAACTGCATCAAGAAGTGAACCTGGTCGTCATGCGGGCC | |
| | ACACAGCTGCAGAAGAATCTGACCTGCGAAGTGTGGGGCCCTACA | |
| | AGCCCTAAACTGATGCTGAGCCTGAAGCTGGAAAACAAAGAAGCC | |
| | AAGGTTTCCAAGCGCGAGAAGGCCGTGTGGGTGTTGAATCCTGAA | |
| | GCCGGCATGTGGCAGTGCCTGCTGAGCGATATCCCTAGCATCGTG | |
| | CTGGGAATCCTGCTGCTGGGCAGCCTGATCTTTATCGCCTTCATC | |
| | CTGCTGAGGATCAAGGGCAAATACGCCCTGCCTGTGATGGTCAAC | |
| | CACCAGCATCTGCCCACAACAATCCCAGCCGGCAGCAATAGCTAC | |
| | CAGCCTGTGCCTATCACAATCCCCAAAGAAGTGTTCATGCTGCCC | |
| | ATCCAGGTTCAGGCCCCACCTCCTGAGGATAGCGATAGCGGCAGC | |
| | GATTCCGACTACGAGCACTACGATTTCAGCGCCCAGCCTCCTGTG | |
| | GCTCTGACCACCTTCTACAACAGCCAGAGACACAGAGTGACCGAC | |
| | GAGGAAGTGCAGCAGAGCAGATTCCAGATGCCTCCTCTGGAAGAA | |
| | GGCCTGGAAGAACTGCACGCCTCTCACATCCCTACAGCCAATCCT | |
| | GGCCACTGCATCACCGATCCTCCATCTCTGGGACCCCAGTATCAC | |
| | CCCAGAAGCAATAGCGAGTCTAGCACCAGCAGCGGCGAGGACTAC | |
| | TGCAACAGCCCTAAGTCTAAGCTGCCACCTTGGAACCCTCAGGTG | |
| | TTCAGCAGCGAGAGAAGCAGCTTTCTCGAGCAGCCTCCTAACCTG | |
| | GAACTGGCCGGAACACAGCCTGCCTTTTCTGCTGGACCTCCTGCC | |
| | GATGATAGCAGCAGCACATCCAGCGGCGAATGGTATCAGAACTTC | |
| | CAGCCACCTCCTCAGCCTCCAAGCGAGGAACAGTTTGGCTGTCCT | |
| | GGCTCTCCATCACCTCAGCCTGACAGCACCGACAACGACGACTAC | |
| | GATGACATCAGCGCCGCT | |
| CD6 D3.3 | ATGTGGCTGTTCTTTGGCATCACCGGACTGCTGACAGCCGCTCTG | 60 |
| | TCTGGACATCCTTCTCCAGCTCCACCTGACCAGCTGAATACCAGC | |
| | TCTGCCGAGTCTGAGCTGTGGGAGCCTGGATGTTCTGAGCACCAG | |
| | TCTTGGAGACTGACCGGCGGAGCCGATAGATGTGAAGGACAGGTC | |
| | GAGGTGCACTTCAGAGGCGTGTGGAACACCGTGTGCGACAGCGAG | |
| | TGGTATCCTTCTGAGGCCAAGGTGCTGTGTCAGTCTCTCGGATGT | |
| | GGCACCGCCGTGGAAAGACCTAAGGGACTGCCTCACAGCCTGAGC | |
| | GGCAGAATGTACTACAGCTGCAACGGCGAGGAACTGACCCTGAGC | |
| | AACTGCAGCTGGCGGTTCAACAACAGCAACCTGTGTAGCCAGAGC | |
| | CTGGCCGCCAGAGTGCTGTGTTCTGCCAGCAGAAGCCTGCACAAC | |
| | CTGTCTACACCTGAGGTGCCAGCCTCTGTGCAGACCGTGACAATC | |
| | GAGTCTAGCGTGACCGTGAAGATCGAGAACAAAGAAAGCCGCGAG | |
| | CTGATGCTGCTGGAAGATCCTGCCGAGCCTAAGAGCCCTGACAAG | |
| | ACCCACACATGCCCTCCATGTCCTGCTCCAGAACTGCTCGGCGGA | |
| | CCCTCCGTTTTCCTGTTTCCACCTAAGCCTAAGGACACCCTGATG | |
| | ATCAGCAGAACCCCTGAAGTGACCTGCGTGGTGGTGGATGTGTCT | |
| | CACGAGGACCCCGAAGTGAAGTTCAATTGGTACGTGGACGGCGTG | |
| | GAAGTGCACAACGCCAAGACCAAGCCTAGAGAGGAACAGTACAAC | |
| | AGCACCTACAGAGTGGTGTCCGTGCTGACCGTGCTGCACCAGGAT | |
| | TGGCTGAACGGCAAAGAGTACAAGTGCAAGGTGTCCAACAAGGCC | |
| | CTGCCTGCTCCTATCGAAAAGACCATCAGCAAGGCCAAGGGCCAG | |
| | CCAAGAGAACCCCAGGITTACACCCTGCCTCCAAGCAGAGATGAG | |
| | CTGACCAAGAACCAGGTGTCCCTGACCTGCCTGGTCAAGGGCTTC | |
| | TACCCTAGCGACATTGCCGTGGAATGGGAGAGCAATGGCCAGCCT | |
| | GAGAACAACTACAAGACCACACCTCCTGTGCTGGACAGCGACGGC | |
| | TCATTCTTCCTGTACAGCAAGCTGACAGTGGACAAGAGCAGATGG | |
| | CAGCAGGGCAACGTGTTCAGCTGTTCTGTGATGCACGAGGCCCTG | |
| | CACAATCACTACACCCAGAAGTCCCTGTCTCTGAGCCCAGGCAAG | |
| | AAGGACCCTAAGATCCCTAGCATCGTGCTGGGCATCCTGCTGCTG | |
| | GGCTCCCTGATCTTTATCGCCTTTATCCTGCTGAGGATCAAGGGC | |
| | AAATACGCCCTGCCAGTGATGGTCAACCACCAGCATCTGCCTACA | |
| | ACAATCCCAGCCGGCAGCAACTCTTACCAGCCTGTGCCAATCACA | |
| | ATCCCCAAAGAGGTGTTCATGCTGCCCATCCAGGTTCAGGCCCCA | |
| | CCTCCTGAGGATAGCGATAGCGGCAGCGATTCCGACTACGAGCAC | |
| | TACGACTTTAGCGCCCAGCCTCCTGTGGCTCTGACCACCTTCTAC | |
| | AACAGCCAGAGACACAGAGTGACCGACGAGGAAGTGCAGCAGAGC | |
| | AGATTCCAGATGCCTCCTCTGGAAGAAGGCCTGGAAGAACTGCAC | |
| | GCCTCTCACATCCCTACCGCCAATCCAGGCCACTGCATCACCGAT | |
| | CCTCCATCTCTGGGACCCCAGTATCACCCCAGAAGCAATAGCGAG | |
| | AGCAGCACCTCTAGCGGCGAGGACTACTGCAACAGCCCTAAGTCT | |
| | AAGCTGCCACCTTGGAATCCCCAGGTGTTCAGCAGCGAGAGAAGC | |
| | AGCTTTCTGGAACAGCCTCCTAACCTGGAACTGGCCGGAACACAG | |
| | CCTGCCTTTTCTGCTGGACCTCCTGCCGATGATAGCAGCTCTACA | |
| | AGCAGCGGCGAATGGTATCAGAACTTCCAGCCACCTCCTCAGCCA | |
| | CCTAGCGAAGAACAGTTTGGCTGTCCTGGCAGCCCATCTCCACAG | |
| | CCAGACAGCACCGACAACGACGACTACGATGACATCAGCGCCGCT | |
| CD6 AD3 | ATGTGGCTCTTCTTCGGGATCACTGGATTGCTGACGGCAGCCCTC | 61 |
| | TCAGGTCATCCATCTCCAGCCCCACCTGACCAGCTCAACACCAGC | |
| | AGTGCAGAGAGTGAGCTCTGGGAGCCAGGGGAGCGGCTTCCGGTC | |
| | CGTCTGACAAACGGGAGCAGCAGCTGCAGCGGGACGGTGGAGGTG | |
| | CGGCTCGAGGCGTCCTGGGAGCCCGCGTGCGGGGCGCTCTGGGAC | |
| | AGCCGCGCCGCCGAGGCCGTGTGCCGAGCACTGGGCTGCGGCGGG | |
| | GCGGAGGCCGCCTCTCAGCTCGCCCCGCCGACCCCTGAGCTGCCG | |
| | CCCCCGCCTGCAGCCGGGAACACCAGCGTAGCAGCTAATGCCACT | |
| | CTGGCCGGGGCGCCCGCCCTCCTGTGCAGCGGCGCCGAGTGGCGG | |
| | CTCTGCGAGGTGGTGGAGCACGCGTGCCGCAGCGACGGGAGGCGG | |
| | GCCCGTGTCACCTGTGCAGAGAACCGCGCGCTGCGCCTGGTGGAC | |
| | GGTGGCGGCGCCTGCGCCGGCCGCGTGGAGATGCTGGAGCATGGC | |
| | GAGTGGGGATCAGTGTGCGATGACACTTGGGACCTGGAGGACGCC | |
| | CACGTGGTGTGCAGGCAACTGGGCTGCGGCTGGGCAGTCCAGGCC | |
| | CTGCCCGGCTTGCACTTCATGCCCGGCCGCGGGCCTATCCACCGG | |
| | GACCAGGTGAACTGCTCGGGGGCCGAAGCTTACCTGTGGGACTGC | |
| | CCGGGGCTGCCAGGACAGCACTACTGCGGCCACAAAGAGGACGCG | |
| | GGCGTGGTGTGCTCAGCTTCCCGGAGTTTGCACAATCTGTCCACT | |
| | CCCGAAGTCCCTGCAAGTGTTCAGACAGTCACTATAGAATCTTCT | |
| | GTGACAGTGAAAATAGAGAACAAGGAATCTCGGGAGCTAATGCTC | |
| | CTCATCCCCTCCATCGTTCTGGGAATTCTCCTCCTTGGCTCCCTC | |
| | ATCTTCATAGCCTTCATCCTCTTGAGAATTAAAGGAAAATATGCC | |
| | CTCCCCGTAATGGTGAACCACCAGCACCTACCCACCACCATCCCG | |
| | GCAGGGAGCAATAGCTATCAACCGGTCCCCATCACCATCCCCAAA | |
| | GAAGTTTTCATGCTGCCCATCCAGGTCCAGGCCCCGCCCCCTGAG | |
| | GACTCAGACTCTGGCTCGGACTCAGACTATGAGCACTATGACTTC | |
| | AGCGCCCAGCCTCCTGTGGCCCTGACCACCTTCTACAATTCCCAG | |
| | CGGCATCGGGTCACAGATGAGGAGGTCCAGCAAAGCAGGTTCCAG | |
| | ATGCCACCCTTGGAGGAAGGACTTGAAGAGTTGCATGCCTCCCAC | |
| | ATCCCAACTGCCAACCCTGGACACTGCATTACAGACCCGCCATCC | |
| | CTGGGCCCTCAGTATCACCCGAGGAGCAACAGTGAGTCGAGCACC | |
| | TCTTCGGGGGAGGATTACTGCAATAGTCCCAAAAGCAAGCTGCCT | |
| | CCATGGAACCCCCAGGTGTTTTCTTCAGAGAGGAGTTCCTTCCTG | |
| | GAGCAGCCCCCAAACTTGGAGCTGGCCAGCACCCAGCCAGCCTTT | |
| | TCAGCAGGGCCCCCGGCTGATGACAGCTCCAGCACCTCATCCGGG | |
| | GAGTGGTACCAGAACTTCCAGCCACCACCCCAGCCCCCTTCGGAG | |
| | GAGCAGTTTGGCTGTCCAGGGTCCCCCAGCCCTCAGCCTGACTCC | |
| | ACCGACAACGATGACTACGATGACATCAGCGCAGCC | |
| dsRed | GGCGACAACACCGAGGACGTGATCAAAGAATTCATGCAGTTCAAA | 62 |
| Monomer | GTGCGGATGGAAGGCAGCGTGAACGGCCACTACTTCGAGATTGAA | |
| | GGCGAAGGCGAGGGCAAGCCTTACGAGGGAACACAGACAGCCAAG | |
| | CTGCAAGTGACAAAAGGCGGCCCTCTGCCTTTTGCCTGGGACATT | |
| | CTGAGCCCTCAGTTCCAGTACGGCAGCAAGGCCTATGTGAAGCAC | |
| | CCCGCCGACATTCCCGACTACATGAAGCTGAGCTTCCCCGAGGGC | |
| | TTCACCTGGGAGAGATCCATGAATTTCGAGGACGGCGGCGTGGTG | |
| | GAAGTCCAGCAAGATAGTTCTCTGCAGGACGGCACCTTCATCTAC | |
| | AAAGTGAAGTTCAAGGGCGTGAACTTCCCCGCTGATGGCCCCGTG | |
| | ATGCAGAAAAAGACAGCCGGATGGGAGCCCTCCACCGAGAAGCTT | |
| | TATCCTCAAGATGGCGTGCTGAAGGGCGAGATCTCTCACGCCCTG | |
| | AAGCTGAAGGATGGCGGCCACTATACCTGCGACTTCAAGACCGTG | |
| | TACAAGGCCAAGAAACCCGTGCAGCTGCCCGGCAACCACTACGTG | |
| | GACAGCAAGCTGGACATCACCAACCACAACGAGGATTACACAGTG | |
| | GTGGAACAGTACGAGCACGCCGAGGCCAGACACTCTGGATCTCAG | |

### 1. Mutation

Changes can be introduced by mutation into a nucleic acid, thereby leading to changes in the amino acid sequence of a polypeptide (e.g., an antibody or antibody derivative, chimeric antigen receptor, etc.) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### 2. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### III. Cancer Therapy

In some aspects, the disclosed methods comprise administering a cancer therapy to a subject, individual, or patient. In some embodiments, the disclosed methods are not limited to use as a cancer therapeutic, but may also be utilized in any immunotherapy where modulation of the avidity of an immune effector cell is desired, such as but not limited to, autoimmune disease and/or infectious disease (bacterial, viral, protozoan, etc), The cancer therapy may be chosen based on an expression level measurement, alone or in combination with a clinical risk score calculated for the subject. The cancer therapy may be chosen based on a genotype of a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is breast cancer. In some aspects, the cancer is HER2+ breast cancer.

In some aspects the cancer is brain cancer. In some aspects, the cancer is glioblastoma and/or medulloblastoma.

In some aspects, the cancer expresses relatively low tumor associated antigen (TAA) levels relative to other cancers expressing the same TAA. In some such embodiments, use of an engineered avidity switch that increases immune synapse avidity may be appropriate.

In some aspects, the cancer expresses relatively high tumor associated antigen (TAA) levels relative to other cancers expressing the same TAA. In some such embodiments, use of an engineered avidity switch that decreases immune synapse avidity may be appropriate.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### B. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy,” “checkpoint blockade therapy,” “immune checkpoint therapy,” “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4 or CTLA4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8⁺ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4 Th1 and CD8 Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4 Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of CD3, B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

As disclosed herein, in certain embodiments, immune effector cells expressing a tumor antigen targeting molecule may be further modified to express a polynucleotide and/or polypeptide of the immediate disclosure, such as but not limited to, polypeptides CD6 D3.1 (SEQ ID NO: 22), CD6 D3.2 (SEQ ID NO: 23), CD6 D3.3 (SEQ ID NO: 24), and/or CD6 ΔD3 (SEQ ID NO: 25).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive Cell Therapy

Adoptive cell therapy is a form of passive immunization by the transfusion of immune cells, such as T cells, NK cells, or other immune cells (also called “adoptive cell transfer”). Immune cells used for adoptive cell therapy include those found in normal tissue and those found in tumor tissue (where they are known as tumor infiltrating immune cells or tumor infiltrating lymphocytes). Although tumor infiltrating immune cells can attack a tumor, the environment within the tumor is generally highly immunosuppressive, preventing immune-mediated tumor death.

Multiple ways of producing and obtaining tumor targeted immune cells have been developed. Immune cells specific to a tumor antigen can be removed from a tumor sample or filtered from blood. Subsequent activation and culturing may be performed ex vivo, with the results reinfused. Activation can take place through gene therapy, by exposing the immune cells to tumor antigens, or by other methods known in the art.

As disclosed herein, immune cells having reduced or no expression of PRKCD may have enhanced anti-tumor immunity. Accordingly, in certain aspects, an adoptive cell therapy of the disclosure comprises administration of immune cells that do not express, or have reduced expression of, PRKCD. For example, an adoptive cell therapy of the disclosure may comprise obtaining immune cells from a subject, eliminating PRKCD gene expression in the cells (e.g., by generating a deletion or other mutation in the PRCKD gene), and administering the cells to the same subject or a different subject.

### 7. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### D. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### E. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### F. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### IV. Cellular Therapies

Aspects of the present disclosure include cellular therapies, including engineering and formulating cellular therapies as well as using such therapies for treatment of various conditions such as cancer.

### A. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

### C. Pharmaceutical Compositions

In certain aspects, the compositions or agents for use in the methods, such as therapeutic immune cells such as CAR T cells, CAR NK cells, etc., are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as a tumor) or systemic delivery, in solid, semi-solid, gel, liquid or gaseous forms such as tablets, capsules, powders, granules, ointments, solutions, depositories, inhalants and injections allowing for oral, parenteral or surgical administration.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### V. Formulations and Culture of the Cells

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21™ supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous TCR, which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FR, GD2, G250/CAIX, GD3, Glypican-3 (GPC3), Her2, IL-13R 2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### A. Chimeric Antigen Receptors

### 1. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane. Generally, the signal peptide natively attached to the amino-terminal most component is used (e.g., in an scFv with orientation light chain-linker-heavy chain, the native signal of the light-chain is used).

In certain embodiments, engineered avidity switch polypeptides may comprise one or more signal peptides. In some embodiments, a signal peptide may have a functional impact on the expression, localization, and/or activity of a polypeptide comprising the same. In some embodiments, a signal peptide sequence may be a CD6 derived sequence.

In some embodiments, the signal peptide is cleaved after passage of the endoplasmic reticulum (ER), i.e., is a cleavable signal peptide. In some embodiments, a restriction site is at the carboxy end of the signal peptide to facilitate cleavage.

### 2. Antigen Binding Domain

Polypeptides of the present disclosure may comprise one or more antigen binding domains. An “antigen binding domain” describes a region of a polypeptide capable of binding to an antigen under appropriate conditions. In some embodiments, an antigen binding domain is a single-chain variable fragment (scFv) based on one or more antibodies (e.g., HER2 antibodies). In some embodiments, an antigen binding domain comprise a variable heavy (VH) region and a variable light (VL) region, with the VH and VL regions being on the same polypeptide. In some embodiments, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some embodiments, the KD of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

### 3. Peptide Spacer

A peptide spacer, such as an extracellular spacer may link an antigen-binding domain to a transmembrane domain. In some embodiments, a peptide spacer is flexible enough to allow the antigen-binding domain to orient in different directions to facilitate antigen binding. In one embodiment, the spacer comprises the hinge region from IgG. In some embodiments, the spacer comprises or further comprises the CH2CH3 region of immunoglobulin and portions of CD3.

In certain embodiments, engineered avidity switch polypeptides may comprise one or more hinges and/or linker sequences. In some embodiments, a hinge and/or linker sequence may have a functional impact on the expression, localization, and/or activity of a polypeptide comprising the same. In some embodiments, a hinge and/or linker sequence may be a CD6 derived sequence.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### 4. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

In certain embodiments, engineered avidity switch polypeptides may comprise a transmembrane domain sequence. In some embodiments, a transmembrane domain sequence may have a functional impact on the expression, localization, and/or activity of a polypeptide comprising the same. In some embodiments, a transmembrane domain sequence may be a CD6 derived sequence.

Any transmembrane domain that provides for insertion of a polypeptide into the cell membrane of a eukaryotic (e.g., mammalian) cell may be suitable for use. In some embodiments, the transmembrane domain is derived from CD28, CD8, CD4, CD3-zeta (CD32), CD134, or CD7.

### 5. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

In certain embodiments, engineered avidity switch polypeptides may comprise a cytoplasmic signaling domain(s) sequence. In some embodiments, a cytoplasmic signaling domain(s) sequence may have a functional impact on the expression, localization, and/or activity of a polypeptide comprising the same. In some embodiments, a cytoplasmic signaling domain(s) sequence may be a CD6 derived sequence.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some embodiments, a suitable cytoplasmic region can comprise an ITAM motif-containing portion of the full length DAP12 amino acid sequence. In some embodiments, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). In some embodiments, a suitable cytoplasmic region can comprise an ITAM motif-containing portion of the full length FCERIG amino acid sequence.

In some embodiments, the cytoplasmic region is derived from T cell surface glycoprotein CD3 delta chain (also known as CD3D; CD3-DELTA; T3D; CD3 antigen, delta subunit; CD3 delta; CD38; CD3d antigen, delta polypeptide (TiT3 complex); OKT3, delta chain; T cell receptor T3 delta chain; T cell surface glycoprotein CD3 delta chain; etc.). In some embodiments, a suitable cytoplasmic region can comprise an ITAM motif-containing portion of the full length CD3 delta amino acid sequence. In some embodiments, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3-epsilon, T3e, etc.). In some embodiments, a suitable cytoplasmic region can comprise an ITAM motif-containing portion of the full length CD3 epsilon amino acid sequence. In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some embodiments, a suitable cytoplasmic region can comprise an ITAM motif-containing portion of the full length CD3 gamma amino acid sequence. In some embodiments, the cytoplasmic region is derived from T cell surface glycoprotein CD3 zeta chain (also known as CD3Z, CD35, T cell receptor T3 zeta chain, CD247, CD3-ZETA, CD3H, CD3Q, T3Z, TCRZ, etc.). In some embodiments, a suitable cytoplasmic region can comprise an ITAM motif-containing portion of the full length CD3 zeta amino acid sequence.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some embodiments, a suitable cytoplasmic region can comprise an ITAM motif-containing portion of the full length CD79A amino acid sequence.

### 6. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDw137; ILA; etc.). In some embodiments, the costimulatory region is derived from an intracellular portion of the transmembrane protein CD28 (also known as Tp44). In some embodiments, the costimulatory region is derived from an intracellular portion of the transmembrane protein ICOS (also known as AILIM, CD278, and CVID1). In some embodiments, the costimulatory region is derived from an intracellular portion of the transmembrane protein OX-40 (also known as TNFRSF4, RP5-902P8.3, ACT35, CD134, OX40, TXGP1L). In some embodiments, the costimulatory region is derived from an intracellular portion of the transmembrane protein BTLA (also known as BTLA1 and CD272). In some embodiments, the costimulatory region is derived from an intracellular portion of the transmembrane protein CD27 (also known as S 152, T14, TNFRSF7, and Tp55). In some embodiments, the costimulatory region is derived from an intracellular portion of the transmembrane protein CD30 (also known as TNFRSF8, DIS166E, and Ki-1). In some embodiments, the costimulatory region is derived from an intracellular portion of the transmembrane protein GITR (also known as TNFRSF18, RP5-902P8.2, AITR, CD357, and GITR-D). In some embodiments, the costimulatory region derived from an intracellular portion of the transmembrane protein HVEM (also known as TNFRSF14, RP3-395M20.6, ATAR, CD270, HVEA, HVEM, LIGHTR, and TR2).

### 7. Peptide Linkers

In some embodiments, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G) n, glycine-serine polymers (including, for example, (GS)n, (GSGGS)n (SEQ ID NO: 63), (G4S)n (SEQ ID NO: 71), and (GGGS)n (SEQ ID NO: 64), where n is an integer of at least one. In some embodiments, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO: 65), GGSGG (SEQ ID NO: 66), GSGSG (SEQ ID NO: 67), GSGGG (SEQ ID NO: 68), GGGSG (SEQ ID NO: 69), or GSSSG (SEQ ID NO: 70).

### B. Cells

Certain embodiments relate to cells comprising polypeptides or nucleic acids of the disclosure. In some embodiments the cell is an immune cell or a T cell. “T cell” includes all types of immune cells expressing CD3 including, but not limited to, T-helper cells, invariant natural killer T (INKT) cells, cytotoxic T cells, T-regulatory cells (Tregs), and gamma-delta T cells. The T cell may refer to a CD4+ or CD8+ T cell. In some aspects, the immune cell is a natural killer (NK) cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Investigation of the Role of CD6 for Immune Cell Avidity Modulation

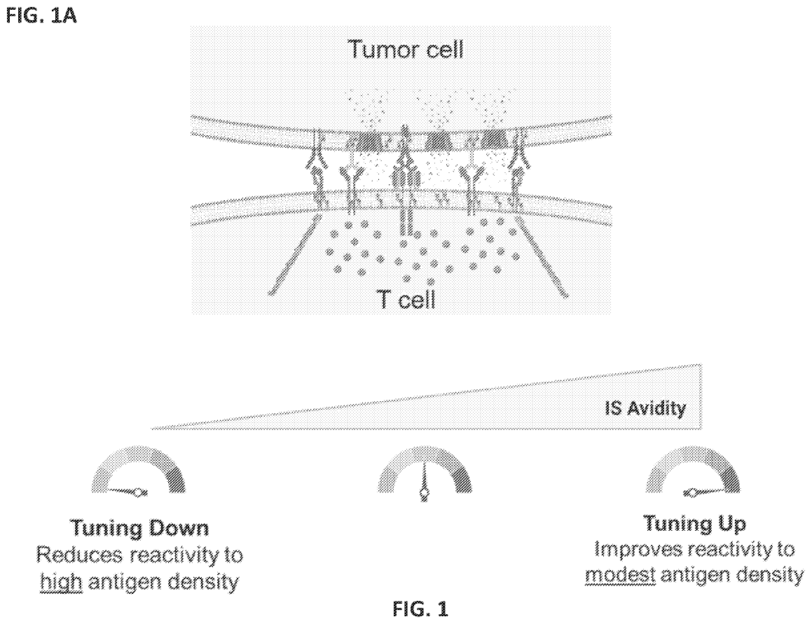

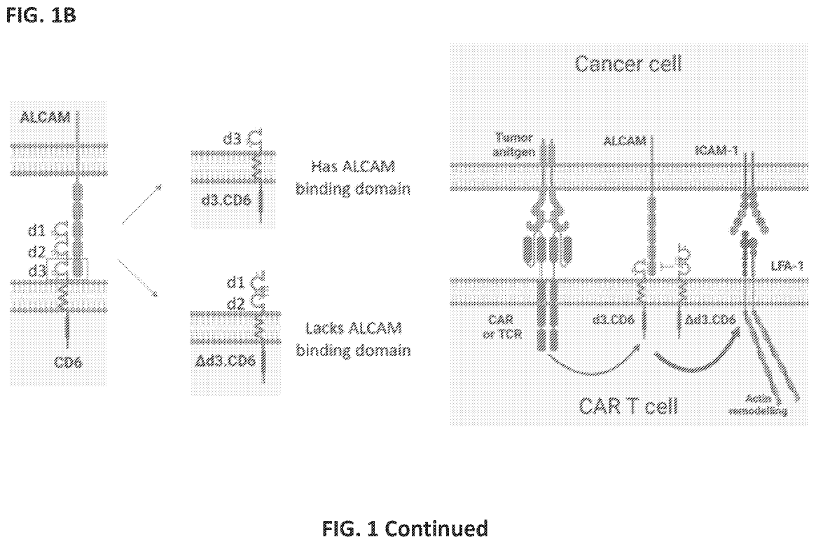

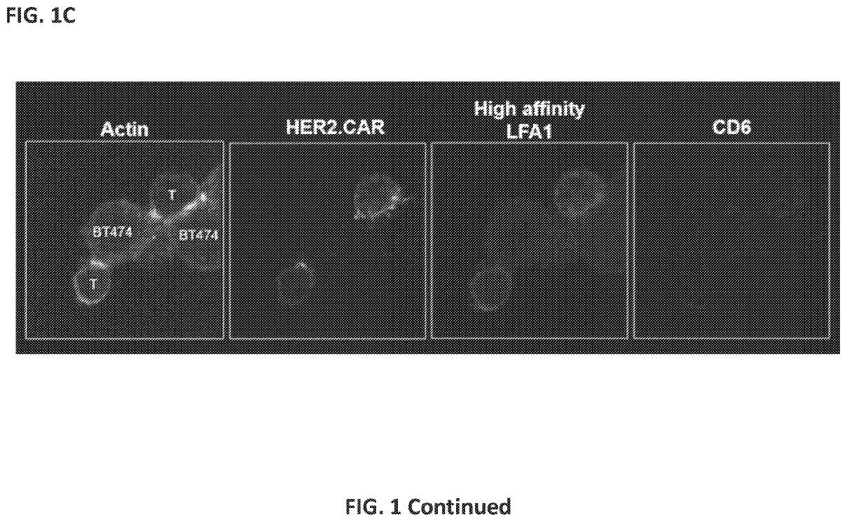

As shown in FIGS. 1A-1C, the immune synapse strength (avidity) of immune cells can be modulated to tune up or tune down immune cell activity. (1A) Is a graphic depicting a tumor cell in contact with an immune effector cell (e.g., T cell), and various binding and signaling actions that can occur & can potentially be modulated. (1B) Is a graphic depicting binding of CD6 extracellular (EC) domains with the target protein ALCAM (left panels), CD6 EC domain 3 (D3) is required for CD6-ALCAM interactions & subsequent signaling cascades/effects (e.g., LFA-1 inside-out activation, recruitment to the immune synapse, and/or actin remodeling), and loss of D3 abolishes CD6-ALCAM binding & potentially inhibits endogenous CD6-ALCAM interactions, e.g. in a dominant negative fashion. (1C) Confocal microscopy immunofluorescence images showing CD6 enriched at effector cell (e.g., “T” cells, e.g., T cells comprising Chimeric Antigen Receptor (CAR) targeting HER2 antigens) tumor cell (e.g. BT474, a HER2-amplified breast cancer cell line) Chimeric Antigen Receptor Immune Synapse (CARIS) sites, where colocalizations of actin, HER2 CARs (GFP tagged), CD6, and activated (high affinity) LFA-1 are apparent.

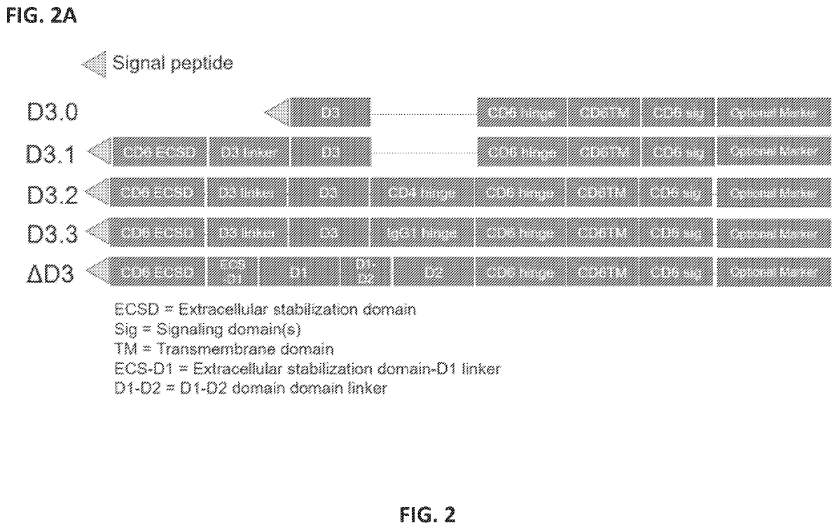

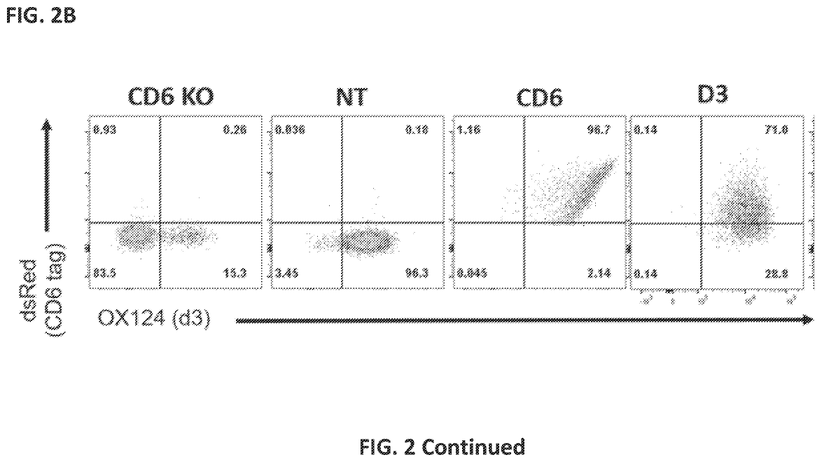

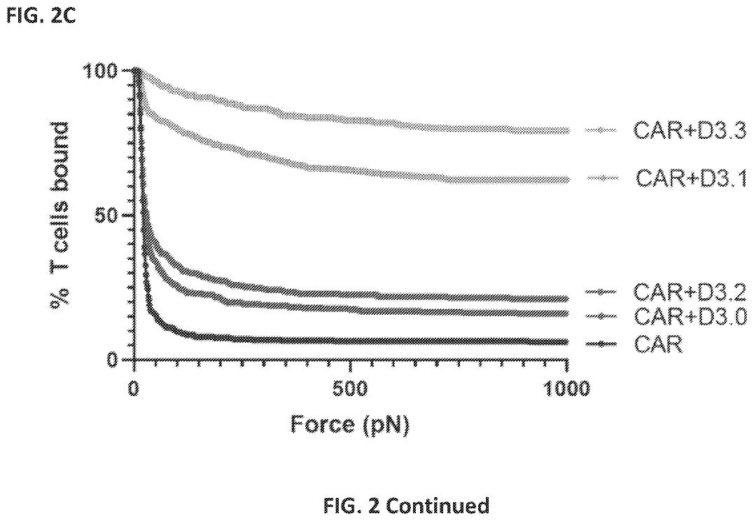

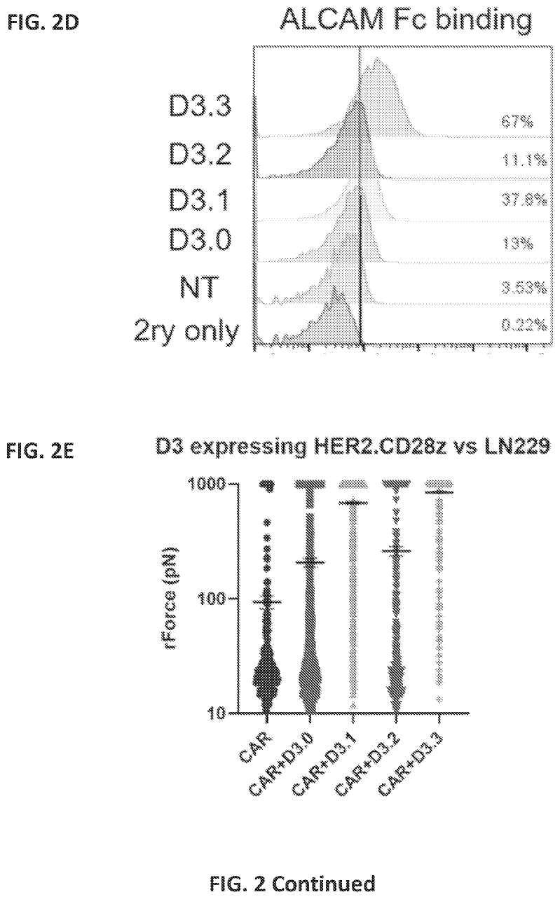

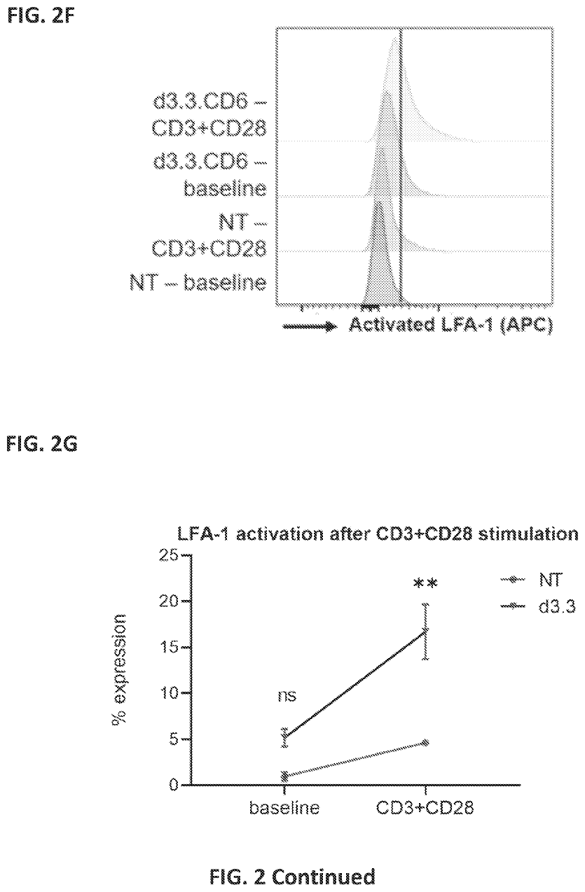

As shown in FIGS. 2A-2G, engineered avidity switch polypeptides were utilized to alter immune effector cell functions. (2A) Is a series of graphics representing exemplary engineered avidity switch polypeptides described herein (e.g., SEQ ID NOs: 21-25). Polypeptides may optionally comprise a marker molecule, such as a fluorophore (e.g., dsRed Mono). (2B) Are representative flow cytometry plots depicting CD6 expression in exemplary transgenic immune cells described herein. (2C) Depicts the percentage decrease in transgenic T cells bound to target LN229 cells upon applying a force ramp of 1000 pico Newtons (pN). Exemplary immune effector cells expressed a HER2.CD28z CAR with or without the noted engineered avidity switch polypeptides (D3.0, D3.1, D3.2, and D3.3 respectively). (2D) Depicts exemplary flow cytometry results showing binding of soluble ALCAM by transgenic immune effector cells expressing the noted engineered avidity switch polypeptides; NT=non-transgenic control; 2ry only=secondary antibody only control. (2E) Depicts the distribution of force in pN needed to detach individual transgenic single cell T cells from their target LN229 cells. (2F) & (2G) Depicts exemplary representative flow cytometry results (2F) and associated quantification (2G) showing the pattern of LFA-1 activation in transgenic cells expressing construct D3.3 after activation with alpha-CD3 and alpha-CD28 antibodies; NT=non-transgenic (representative of 2 experiments); showing statistical differences; parametric T-test with Holm-Sidak adjustment for multiple comparisons, ns=non-significant; **=p-value<0.01.

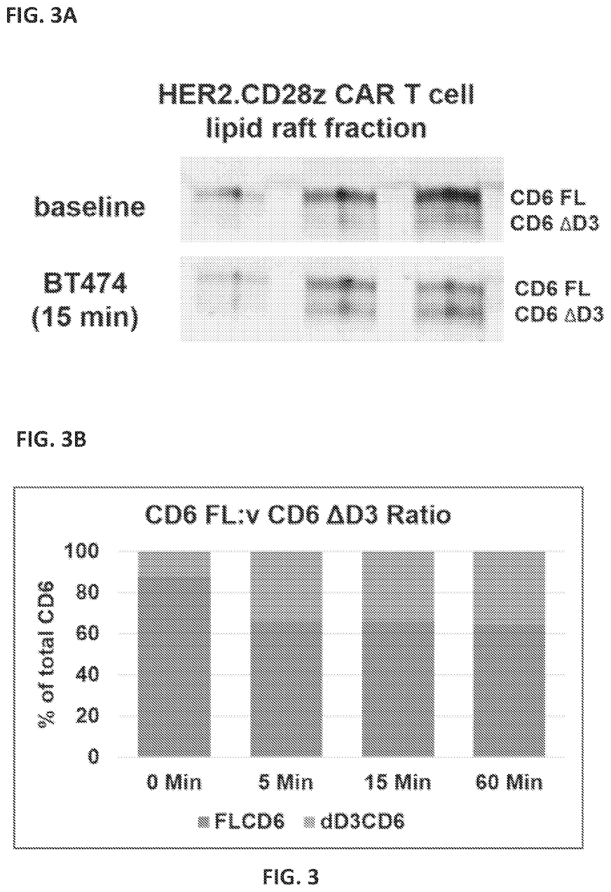

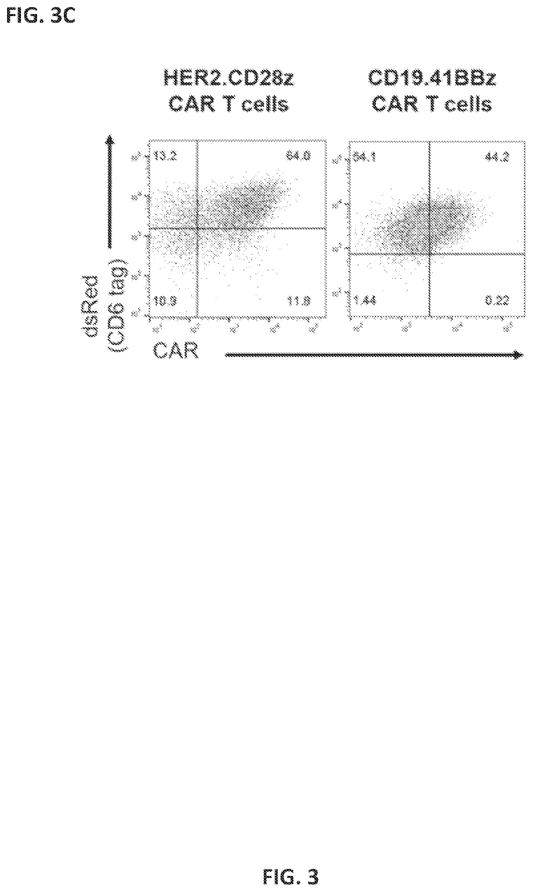

As shown in FIGS. 3A-3C, expression of a dominant negative CD6 polypeptide in immune effector cells was found to reduce avidity while increasing cell cytotoxicity. (3A) Depicts representative western blotting of endogenous full-length CD6 (CD6 FL) and dominant negative CD6 (CD6 ΔD3) in primary HER2 CAR T cells at baseline and 15 minutes post-exposure to BT474 cancer cells. (3B) Depicts the percentage of CD6 FL and CD6 ΔD3 of total CD6 at 0, 5, 16, and 60 minutes post T cell activation using anti-human CD3 and anti-human CD28 antibodies. (3C) Depicts the percentage of transduced T cells expressing a CAR (HER2.CD28z or CD19.41BBz) and CD6 ΔD3 (dsRed (CD6 tag)).

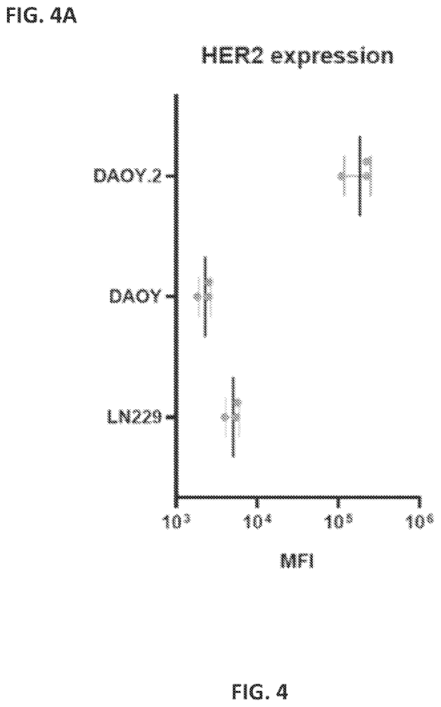

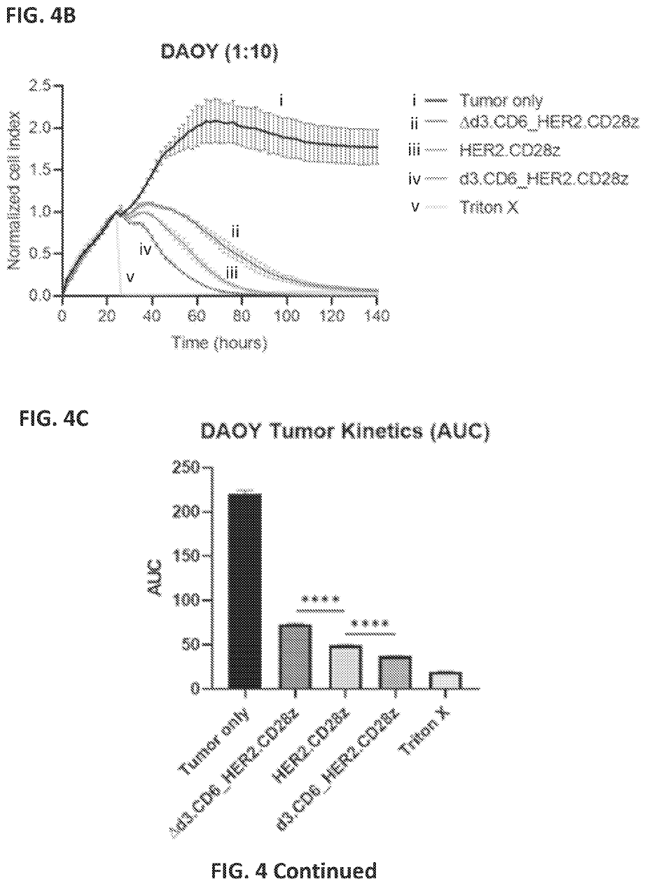

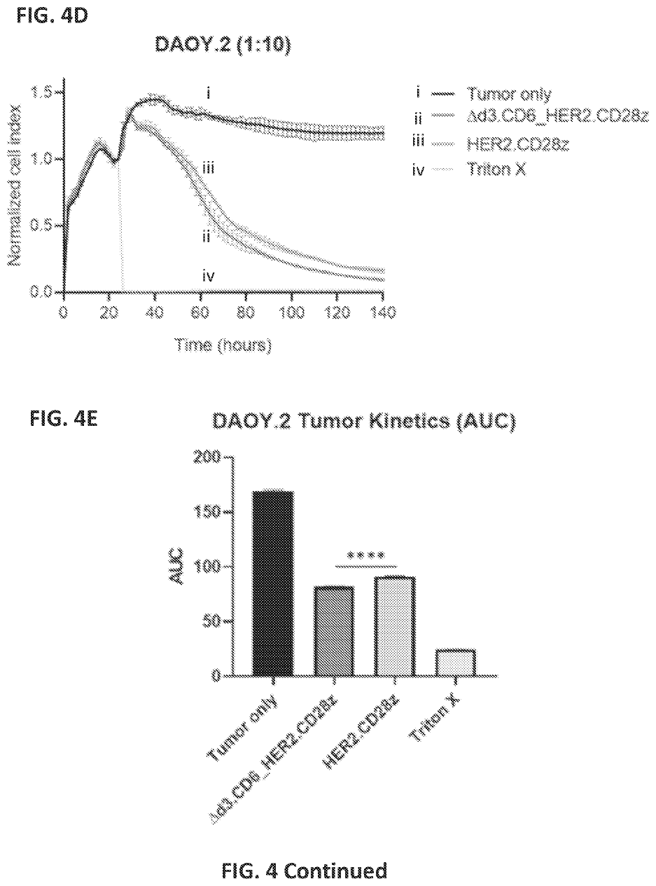

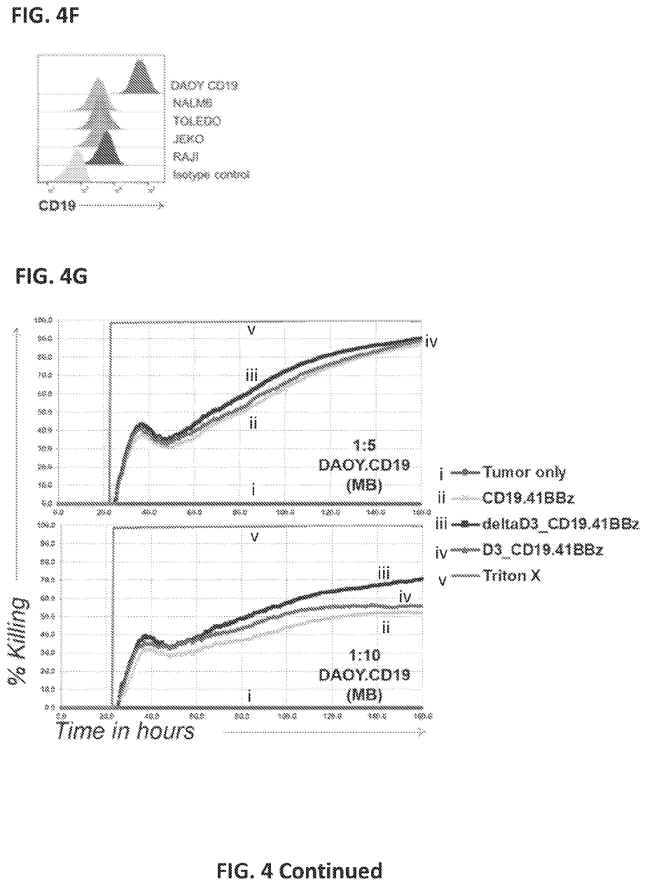

As shown in FIGS. 4A-4G, immune cell cytotoxicity was enhanced through modulation of immune cell avidity by transgenic expression of engineered avidity switch polypeptides. (4A) Depicts the quantitative expression of HER2 as mean fluorescent intensity (MFI) from three representative cancer cell lines (DAOY, DAOY.2, and LN229). (4B) & (4C) Depicts an improvement in killing of DAOY cancer cells by HER2.CD28z CAR T-cells expressing d3.CD6 compared to HER2.CD28z only control cells or dominant negative (ΔD3.CD6) expressing cells (4B) and the representative mean area under the curve (AUC) of each group (4C) showing statistical differences; parametric T-test with Holm-Sidak adjustment for multiple comparisons, **=p-value<0.0001. (4D) & (4E) Depicts an improvement in killing of DAOY.2 cancer cells by HER2.CD28z CAR T-cells expressing dominant negative CD6 (ΔD3.CD6) compared to HER2.CD28z only control cells (4D) and the representative mean area under the curve (AUC) of each group (4E) showing statistical differences; parametric T-test with Holm-Sidak adjustment for multiple comparisons, ****=p-value<0.0001. (4F) Depicts the quantitative expression of CD19 by a variety of cancer cell lines (RAJI, JEKO, TOLEDO, NALM6) in comparison to a transduced DAOY cell line overexpressing CD19 (DAOY CD19). (4G) Depicts improved killing of DAOY CD19 cancer cells by HER2.CD28z CAR T-cells expressing dominant negative CD6 (ΔD3.CD6) compared to HER2.CD28z only control and HER2.CD28z d3 expressing cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references cited herein, to the extent that they provide exemplary procedural or other details supplementary to those set forth herein, are specifically incorporated herein by reference.

What is claimed is:

1. A polynucleotide encoding an engineered avidity switch, comprising an extracellular stabilization domain encoding sequence comprising at least about 80% sequence identity to SEQ ID NOs: 47 or 48, a Scavenger Receptor Cysteine-Rich (SRCR) domain encoding sequence comprising at least about 80% sequence identity to SEQ ID NOs: 39 or 40, a hinge region encoding sequence, a transmembrane domain encoding sequence, and an intracellular signaling region encoding sequence.

2. The polynucleotide of claim 1, further comprising a Cluster of Differentiation 6 (CD6) extracellular domain 2 (d2) and domain 3 (d3) linker region encoding sequence comprising at least about 80% sequence identity to SEQ ID NO: 49.

3. The polynucleotide of claim 1, wherein the hinge region encoding sequence comprises a CD6 hinge region encoding sequence comprising at least about 80% sequence identity to SEQ ID NOs: 41 or 42.

4. The polynucleotide of claim 1, wherein the transmembrane domain encoding sequence comprises a CD6 transmembrane region encoding sequence comprising at least about 80% sequence identity to SEQ ID NOs: 43-46.

5. The polynucleotide of claim 1, wherein the intracellular signaling region encoding sequence comprises a CD6 intracellular signaling region encoding sequence comprising at least about 80% sequence identity to SEQ ID NOs: 28-36.

6. The polynucleotide of claim 1, further comprising a signal peptide encoding sequence, wherein optionally the signal peptide encoding sequence comprises a sequence at least about 80% identical to SEQ ID NOs: 37 or 38.

7. The polynucleotide of claim 1, further comprising a non-CD6 hinge region encoding sequence.

8. The polynucleotide of claim 7, wherein the non-CD6 hinge region encoding sequence comprises a Cluster of Differentiation 4 (CD4) and/or an Immunoglobulin G 1 (IgG1) hinge encoding sequence comprising at least about 80% sequence identity to SEQ ID NOs: 50 and/or 51.

9. The polynucleotide of claim 1, wherein the an extracellular stabilization domain encoding sequence comprises a CD6 extracellular stabilization (ECS) domain encoding sequence comprising at least about 90% sequence identity to SEQ ID NO: 47 or 48.

10. The polynucleotide of claim 1, wherein the SRCR domain encoding sequence comprises a CD6 SRCR domain encoding sequence comprising at least about 90% identity to SEQ ID NO: 39 or 40.

11. The polynucleotide of claim 1, wherein the encoding sequences are operably linked in tandem.

12. The polynucleotide of claim 1, comprising a 5′ to 3′ orientation of:
i) the extracellular stabilization domain encoding sequence,
ii) the SRCR domain encoding sequence,
iii) the hinge region encoding sequence,
iv) the transmembrane domain encoding sequence, and
v) the intracellular signaling region encoding sequence.

13. The polynucleotide of claim 1, wherein the polynucleotide comprises a nucleic acid sequence with at least 80% sequence identity to SEQ ID NOs: 58-60.

14. The polynucleotide of claim 1, wherein the polynucleotide is operably linked to a regulatory sequence.

15. The polynucleotide of claim 14, wherein the regulatory sequence comprises one or more of a promoter, a transcription initiation sequence, an internal ribosome entry site (IRES), an enhancer, an intron, an RNA interference target sequence, a Kozak sequence, splicing regulatory elements, and/or a polyadenylation signal.

16. The polynucleotide of claim 1, wherein the polynucleotide is comprised in a vector and/or integrated into a genome.

17. The polynucleotide of claim 16, wherein the vector is a plasmid, recombinant vector, and/or a viral vector.

18. The viral vector of claim 17, wherein the viral vector comprises a retroviral, lentiviral, adenoviral, or adeno-associated viral packaging vector.

19. A polypeptide encoded by polynucleotide or vector of any one of claims 1 to 18.

20. A polypeptide, comprising an extracellular stabilization domain comprising at least 80% sequence identity to SEQ ID NO: 13, an SRCR domain comprising at least 80% sequence identity to SEQ ID NO: 10, a hinge region, a transmembrane domain, and an intracellular signaling region.

21. The polypeptide of claim 20, further comprising a CD6 d2-d3 linker region comprising at least 80% sequence identity to SEQ ID NO: 14.

22. The polypeptide of claim 20, wherein the hinge region comprises a CD6 hinge region comprising at least 80% sequence identity to SEQ ID NO: 11.

23. The polypeptide of claim 20, wherein the transmembrane domain comprises a CD6 transmembrane region comprising at least 80% sequence identity to SEQ ID NO: 12.

24. The polypeptide of claim 20, wherein the intracellular signaling region comprises a CD6 intracellular signaling region comprising at least 80% sequence identity to SEQ ID NO: 2-8.

25. The polypeptide of claim 20, further comprising a signal peptide comprising at least 80% sequence identity to SEQ ID NO: 9.

26. The polypeptide of claim 20, further comprising a non-CD6 hinge region.

27. The polypeptide of claim 26, wherein the non-CD6 hinge region comprises a CD4 and/or IgG1 hinge comprising at least 80% identity to SEQ ID NOs: 15 or 16.

28. The polypeptide of claim 20, wherein the extracellular stabilization domain comprises a sequence of at least 90% sequence identity to SEQ ID NO: 13.

29. The polypeptide of claim 20, wherein the SRCR domain comprises a CD6 SRCR domain comprising at least 90% identity to SEQ ID NOs: 10.

30. The polypeptide of claim 20, wherein the domains are operably joined in tandem.

31. The polypeptide of claim 20, wherein the polypeptide comprises an N- to C-terminus orientation in the following order:
i) the extracellular stabilization domain,
ii) the SRCR domain,
iii) the hinge region,
iv) transmembrane domain, and
v) intracellular domain.

32. The polypeptide of claim 20, wherein the polypeptide comprises an amino acid sequence comprising at least 80% sequence identity to SEQ ID NOs: 22-24.

33. A method of making a transgenic avid cell expressing a recombinant protein, comprising transfecting, transforming, and/or transducing a cell in vitro and/or ex vivo with the polynucleotide and/or vector of any one of claims 1 to 18.

34. The transgenic avid cell produced according to claim 33.

35. The transgenic avid cell of claim 34, wherein the cell is a mammalian cell.

36. The transgenic avid cell of claim 34, wherein the cell is a mouse and/or human cell.

37. The transgenic avid cell of claim 34, wherein the cell is an immune cell.

38. The transgenic avid cell of claim 37, wherein the immune cell is a T cell, natural killer T cell (NK T cell), macrophage, dendritic cell, and/or natural killer (NK) cell.

39. The transgenic avid cell of claim 38, wherein the immune cell is a T cell or an NK cell.

40. The transgenic avid cell of claim 34, wherein the transgenic avid cell expresses one or more additional transgenes.

41. The transgenic avid cell of claim 40, wherein the one or more additional transgenes comprise a sequence encoding an antigen recognition domain.

42. The transgenic avid cell of claim 41, wherein the sequence encoding an antigen recognition domain encodes an antibody, antibody derivative, chimeric antigen receptor (CAR) and/or T-cell receptor (TCR).

43. The transgenic avid cell of claim 42, wherein the transgenic avid cell expresses a CAR and/or TCR.

44. The CAR and/or TCR expressing transgenic avid cell of claim 43, wherein the CAR and/or TCR targets a Human Epidermal Growth Factor Receptor 2 (Her2) and/or Cluster of Differentiation 19 (CD19) antigen.

45. The CAR and/or TCR expressing transgenic avid cell of claim 44, wherein the CAR comprises a HER2.CD28ζ and/or a CD19.41BBζ CAR.

46. The transgenic avid cell of claim 34, wherein the transgenic cell exhibits greater avidity to a target cell relative to a control cell.

47. The transgenic avid cell of claim 46, wherein the avidity of the transgenic cell to a target cell comprises a greater than two-fold increase relative to a control cell.

48. The transgenic avid cell of claim 46, wherein the avidity of the transgenic cell to a target cell comprises a greater than four-fold increase relative to a control cell.

49. The transgenic avid cell of claim 46, wherein the avidity of the transgenic cell to a target cell comprises a greater than six-fold increase relative to a control cell.

50. The transgenic avid cell of claim 46, wherein the avidity of the transgenic cell to a target cell comprises a greater than eight-fold increase relative to a control cell.

51. The transgenic avid cell of claim 46, wherein the avidity of the transgenic cell to a target cell comprises a greater than ten-fold increase relative to a control cell.

52. The transgenic avid cell of claim 46, wherein the control cell is a non-transgenic cell, and/or a non-avid transgenic cell.

53. The transgenic avid cell of claim 34, wherein the transgenic avid cell exhibits an increase in leukocyte adhesion glycoprotein 1 (LFA-1) recruitment to an immune synapse relative to a control cell.

54. The transgenic avid cell of claim 34, wherein the transgenic avid cell exhibits an increase in activated LFA-1 levels relative to a control cell.

55. The transgenic avid cell of claim 34, wherein the transgenic avid cell exhibits greater killing efficiency of a target cell relative to a control cell.

56. The transgenic avid cell of claim 53, wherein the control cell is a non-transgenic cell, and/or a non-avid transgenic cell.

57. The transgenic avid cell of claim 34, wherein the transgenic avid cell comprises one or more mutations in one or more endogenous genes.

58. A composition comprising the polynucleotide, and/or polypeptide according to any one of the claim 1-18, or 20-32.

59. A composition comprising the avid cell according to claim 34.

60. The composition of claim 58, further comprising a pharmaceutically acceptable carrier.

61. The composition of claim 59, further comprising a pharmaceutically acceptable carrier.

62. A method for treating an individual, comprising administering to the individual in need thereof, a polynucleotide, polypeptide, transgenic avid cell, and/or composition according to any one of claims 1 to 61.

63. A method for treating an individual, comprising administering to the individual in need thereof, the composition of claim 60.

64. The method of claim 63, wherein the individual has, is diagnosed with, and/or is suspected of having: cancer, an autoimmune disorder, a viral infection, neurological disorder, neurodegenerative disorder, and/or a bacterial infection.

65. The method of claim 64, wherein the individual has, is diagnosed with, and/or is suspected of having cancer.

66. The method of claim 65, wherein the cancer has a modest and/or low tumor associated antigen (TAA) expression level relative to other cancers expressing the same TAA.

67. The method of claim 65, wherein the cancer comprises glioblastoma and/or medulloblastoma.

68. The method of claim 65, wherein the cancer comprises glioblastoma multiform.

69. The method of claim 65, wherein the individual has been diagnosed with a cancer having modest or low Her2 TAA expression relative to other cancers expressing Her2 TAA.

70. A method for treating an individual, comprising administering to the individual in need thereof, a transgenic avid cell according to claim 34.

71. The method of claim 70, wherein the individual has, is diagnosed with, and/or is suspected of having: cancer, an autoimmune disorder, a viral infection, neurological disorder, neurodegenerative disorder, and/or a bacterial infection.

72. The method of claim 71, wherein the individual has, is diagnosed with, and/or is suspected of having cancer.

73. The method of claim 72, wherein the cancer has a modest and/or low tumor associated antigen (TAA) expression level relative to other cancers expressing the same TAA.

74. The method of claim 72, wherein the cancer comprises glioblastoma and/or medulloblastoma.

75. The method of claim 72, wherein the cancer comprises glioblastoma multiform.

76. The method of claim 72, wherein the individual has been diagnosed with a cancer having modest or low Her2 TAA expression relative to other cancers expressing Her2 TAA.

77. The method of claim 71, wherein the transgenic avid cell comprises an autologous or allogenic cell.

78. A polynucleotide, comprising an extracellular stabilization domain encoding sequence comprising at least about 80% sequence identity to SEQ ID NO: 47 or 48, one or more extracellular domain encoding sequence, a transmembrane domain encoding sequence, and an intracellular signaling region encoding sequence, wherein the polynucleotide is operably linked to a non-endogenous promoter sequence.

79. The polynucleotide of claim 78, wherein the one or more extracellular domain encoding sequence comprises a sequence comprising at least 80% sequence identity to CD6 extracellular domain 1 (d1) encoding sequence (SEQ ID NO: 53), a sequence comprising at least 80% sequence identity to CD6 extracellular domain 2 (d2) encoding sequence (SEQ ID NO: 55), and/or a sequence comprising at least 80% sequence identity to CD6 extracellular domain 3 (d3) encoding sequence (SEQ ID NOs: 39 or 40).

80. The polynucleotide of claim 79, wherein the one or more extracellular domain encoding sequence comprises a sequence comprising at least 80% sequence identity to CD6 extracellular domain 1 (d1) encoding sequence (SEQ ID NO: 53), a sequence comprising at least 80% sequence identity to CD6 extracellular domain 2 (d2) encoding sequence (SEQ ID NO: 55), and does not comprise a d3 encoding sequence (SEQ ID NOs: 39 or 40).

81. The polynucleotide of claim 79, wherein the d1, d2, and/or d3 encoding sequences comprise engineered mutations to alter the independent and/or combined functions of d1, d2, and/or d3, comprising at least about 80% sequence identity to SEQ ID NOs: 53, 55, 39, and/or 40.

82. The polynucleotide of claim 78, wherein the transmembrane domain encoding sequence comprises a CD6 transmembrane region encoding sequence comprising at least about 80% sequence identity to SEQ ID NOs: 43-46.

83. The polynucleotide of claim 78, wherein the intracellular signaling region encoding sequence comprises a CD6 intracellular signaling region encoding sequence comprising at least about 80% sequence identity to SEQ ID NOs: 28-36.

84. The polynucleotide of claim 83, wherein the CD6 intracellular signaling region has been genetically engineered to modify the function of the CD6 intracellular signaling region relative to wild type full length CD6 protein as represented by SEQ ID NO: 1, comprising at least about 80% sequence identity to SEQ ID NOs: 28-36.

85. The polynucleotide of claim 78, wherein the encoding sequences are operably linked in tandem.

86. The polynucleotide of claim 78, comprising a 5′ to 3′ orientation of:
i) the extracellular stabilization domain encoding sequence,
ii) the one or more extracellular domain encoding sequence(s),
iii) the transmembrane domain encoding sequence, and
iv) the intracellular signaling region encoding sequence.

87. The polynucleotide of claim 86, further comprising a nucleic acid sequence encoding a CD6 hinge region at least 80% identical to SEQ ID NOs: 41 or 42, interspersed between the one or more extracellular domain encoding sequences and the transmembrane domain encoding sequence.

88. The polynucleotide of claim 86, further comprising a nucleic acid sequence encoding a CD6 ECS-D1 linker (SEQ ID NO: 52) interspersed between the extracellular stabilization domain and a first extracellular domain, and a nucleic acid sequence encoding a CD6 D1-D2 linker (SEQ ID NO: 54) comprised between two extracellular domain encoding sequences.

89. The polynucleotide of claim 78, further comprising a signal peptide encoding sequence, wherein optionally the signal peptide encoding sequence comprises a sequence at least about 80% identical to SEQ ID NOs: 37 or 38.

90. The polynucleotide of claim 78, wherein the polynucleotide comprises a nucleic acid sequence comprising at least 80% sequence identity to SEQ ID NO: 61.

91. The polynucleotide of claim 78, wherein the polynucleotide is operably linked to a regulatory sequence.

92. The polynucleotide of claim 91, wherein the regulatory sequence comprises one or more of, a transcription initiation sequence, an internal ribosome entry site (IRES), an enhancer, an intron, an RNA interference target sequence, a Kozak sequence, splicing regulatory elements, and/or a polyadenylation signal.

93. The polynucleotide of claim 78, wherein the polynucleotide is comprised in a vector and/or integrated into a genome.

94. The polynucleotide of claim 93, wherein the vector is a plasmid, recombinant vector, and/or a viral vector.

95. The vector of claim 94, wherein the vector is a viral vector, and the viral vector comprises a retroviral, lentiviral, adenoviral, or adeno-associated viral packaging vector.

96. A polypeptide encoded by the polynucleotide of any one of claims 78 to 95.

97. A polypeptide, comprising an extracellular stabilization domain comprising at least about 80% sequence identity to SEQ ID NO: 13, one or more extracellular domains, a transmembrane domain, and an intracellular signaling region.

98. The polypeptide of claim 97, wherein the one or more extracellular domains comprises an amino acid sequence comprising at least 80% sequence identity to CD6 extracellular domain 1 (d1) (SEQ ID NO: 18), a sequence comprising at least 80% sequence identity to CD6 extracellular domain 2 (d2) (SEQ ID NO: 20), and/or a sequence comprising at least 80% sequence identity to CD6 extracellular domain 3 (d3) (SEQ ID NO: 10).

99. The polypeptide of claim 98, wherein the one or more extracellular domains comprises an amino acid sequence comprising an amino acid sequence comprising at least 80% sequence identity to CD6 extracellular domain 1 (d1) (SEQ ID NO: 18), a sequence comprising at least 80% sequence identity to CD6 extracellular domain 2 (d2) (SEQ ID NO: 20), and does not comprise a CD6 extracellular domain 3 (d3) amino acid sequence (SEQ ID NO: 10).

100. The polypeptide of claim 98, wherein the d1, d2, and/or d3 comprising engineered mutations that modify the independent and/or combined endogenous functions of d1, d2, and/or d3, comprising at least about 80% sequence identity to SEQ ID NOs: 18, 20, and/or 10.

101. The polypeptide of claim 97, wherein the transmembrane domain comprises a CD6 transmembrane region comprising at least about 80% sequence identity to SEQ ID NO: 12.

102. The polypeptide of claim 97, wherein the intracellular signaling region comprises a CD6 intracellular signaling sequence comprising at least about 80% sequence identity to SEQ ID NOs: 2-8.

103. The polypeptide of claim 102, wherein the CD6 intracellular signaling region comprises genetically engineered mutations modifying the function of the CD6 intracellular signaling region relative to endogenous functions, comprising at least about 80% sequence identity to SEQ ID NOs: 2-8.

104. The polypeptide of claim 97, wherein the domains and/or regions are operably linked in tandem.

105. The polypeptide of claim 97, comprising a N- to C-terminus orientation of:
i) the extracellular stabilization domain,
ii) the one or more extracellular domain(s),
iii) the transmembrane domain, and
vi) the intracellular signaling region.

106. The polypeptide of claim 105, further comprising a CD6 hinge region sequence at least 80% identical to SEQ ID NO: 11, interspersed between the one or more extracellular domains and the transmembrane domain.

107. The polypeptide of claim 105, further comprising a CD6 ECS-D1 linker sequence (SEQ ID NO: 17) interspersed between the extracellular stabilization domain and a first extracellular domain, and a CD6 D1-D2 linker sequence (SEQ ID NO: 19) comprised between two extracellular domains.

108. The polypeptide of claim 97, further comprising a signal peptide sequence, wherein optionally the signal peptide sequence comprises a sequence at least about 80% identical to SEQ ID NO: 9.

109. The polypeptide of claim 97, wherein the polypeptide comprises an amino acid sequence comprising at least 80% sequence identity to SEQ ID NO: 25.

110. A method of making a transgenic avidity-weakened cell expressing a recombinant protein, comprising transfecting, transforming, and/or transducing a cell in vitro and/or ex vivo with the polynucleotide and/or vector of any one of claims 78 to 95.

111. The transgenic avidity-weakened cell produced according to claim 110.

112. The transgenic avidity-weakened cell of claim 111, wherein the cell is a mammalian cell.

113. The transgenic avidity-weakened cell of claim 111, wherein the cell is a mouse and/or human cell.

114. The transgenic avidity-weakened cell of claim 111, wherein the cell is an immune cell.

115. The transgenic avidity-weakened cell of claim 114, wherein the immune cell is a T cell, natural killer T cell (NK T cell), macrophage, dendritic cell, and/or natural killer (NK) cell.

116. The transgenic avidity-weakened cell of claim 115, wherein the immune cell is a T cell or an NK cell.

117. The transgenic avidity-weakened cell of claim 111, wherein the transgenic avidity-weakened cell expresses one or more additional transgenes.

118. The transgenic avidity-weakened cell of claim 117, wherein the one or more additional transgenes comprise a sequence encoding an antigen recognition domain.

119. The transgenic avidity-weakened cell of claim 118, wherein the sequence encoding an antigen recognition domain encodes an antibody, antibody derivative, CAR and/or TCR.

120. The transgenic avidity-weakened cell of claim 119, wherein the transgenic avid-weakened cell expresses a CAR and/or TCR.

121. The CAR and/or TCR expressing transgenic avidity-weakened cell of claim 120, wherein the CAR and/or TCR targets a Her2 and/or CD19 antigen.

122. The CAR and/or TCR expressing transgenic avidity-weakened cell of claim 121, wherein the CAR comprises a HER2.CD28ζ and/or a CD19.41BBζ CAR.

123. The transgenic avidity-weakened cell of claim 111, wherein the transgenic avidity-weakened cell exhibits lower avidity to a target cell relative to a control cell, wherein the control cell is a transgenic non-avidity-weakened cell, non-transgenic cell, and/or an avid transgenic cell.

124. The transgenic avidity-weakened cell of claim 123, wherein the avidity of the transgenic avidity-weakened cell to a target cell comprises a greater than two-fold decrease relative to a control cell, wherein the control cell is a transgenic non-avidity-weakened cell, non-transgenic cell, and/or an avid transgenic cell.

125. The transgenic avidity-weakened cell of claim 111, wherein the transgenic avid-weakened cell exhibits a decrease in LFA-1 recruitment to an immune synapse relative to a control cell, wherein the control cell is a transgenic non-avidity-weakened cell, non-transgenic cell, and/or an avid transgenic cell.

126. The transgenic avidity-weakened cell of claim 111, wherein the transgenic avid-weakened cell exhibits a decrease in activated LFA-1 levels relative to a control cell, wherein the control cell is a transgenic non-avidity-weakened cell, non-transgenic cell, and/or an avid transgenic cell.

127. The transgenic avidity-weakened cell of claim 111, wherein the transgenic avid-weakened cell exhibits greater killing efficiency of a target cell relative to a control cell, wherein the control cell is a transgenic non-avidity-weakened cell, non-transgenic cell, and/or an avid transgenic cell.

128. The transgenic avidity-weakened cell of claim 126, wherein the control cell is a transgenic non-avidity-weakened cell, non-transgenic cell, and/or an avid transgenic cell.

129. The transgenic avidity-weakened cell of claim 111, wherein the transgenic avidity-weakened cell comprises one or more mutations in one or more endogenous genes.

130. A composition comprising the polynucleotide and/or polypeptide, according to any one of the claims 78 to 95, or 97 to 109.

131. A composition comprising the transgenic avidity-weakened cell according to claim 111.

132. The composition of claim 130, further comprising a pharmaceutically acceptable carrier.

133. A method for treating an individual, comprising administering to the individual in need thereof, a composition according to claim 132.

134. The method of claim 133, wherein the individual has, is diagnosed with, and/or is suspected of having: cancer, an autoimmune disorder, a viral infection, a neurological disorder, a neurodegenerative disorder, and/or a bacterial infection.

135. The method of claim 134, wherein the individual has, is diagnosed with, and/or is suspected of having cancer.

136. The method of claim 135, wherein the cancer has a medium and/or high TAA expression level relative to other cancers expressing the same TAA.

137. The method of claim 134, wherein the cancer comprises glioblastoma and/or medulloblastoma.

138. The method of claim 134, wherein the cancer comprises glioblastoma multiform.

139. The method of claim 134, wherein the individual has been diagnosed with a cancer having medium and/or high Her2 TAA expression relative to other cancers expressing Her2 TAA.

140. The composition of claim 131, further comprising a pharmaceutically acceptable carrier.

141. A method for treating an individual, comprising administering to the individual in need thereof, a composition according to claim 140.

142. The method of claim 141, wherein the individual has, is diagnosed with, and/or is suspected of having: cancer, an autoimmune disorder, a viral infection, a neurological disorder, a neurodegenerative disorder, and/or a bacterial infection.

143. The method of claim 141, wherein the individual has, is diagnosed with, and/or is suspected of having cancer.

144. The method of claim 143, wherein the cancer has a medium and/or high TAA expression level relative to other cancers expressing the same TAA.

145. The method of claim 143, wherein the cancer comprises glioblastoma and/or medulloblastoma.

146. The method of claim 143, wherein the cancer comprises glioblastoma multiform.

147. The method of claim 143, wherein the individual has been diagnosed with a cancer having medium and/or high Her2 TAA expression relative to other cancers expressing Her2 TAA.

148. The method of claim 143, wherein the transgenic avidity-weakened cell comprises an autologous or allogenic cell.


SEQUENCE LISTING (71 sequences)

SEQ ID NO: 1 - protein, 668 aa, synthetic construct
MWLFFGITGLLTAALSGHPSPAPPDQLNTSSAESELWEPGERLPVRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCAENRALRLVDGGGACAGRVEMLEHGEWGSVCDDTWDLEDAHVVCRQLGCGWAVQALPGLHFTPGRGPIHRDQVNCSGAEAYLWDCPGLPGQHYCGHKEDAGAVCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 2 - protein, 245 aa, synthetic construct
LRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 3 - protein, 213 aa, synthetic construct
LRIKGKYVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 4 - protein, 172 aa, synthetic construct
LRIKGKYDSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 5 - protein, 178 aa, synthetic construct
LRIKGKYVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSGSPSPQPDSTDNDDYDDISAA

SEQ ID NO: 6 - protein, 169 aa, synthetic construct
LRIKGKYALPVMVNHQHLPTTIPAGSNSYQPVPITIPKEDSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSGSPSPQPDSTDNDDYDDISAA

SEQ ID NO: 7 - protein, 203 aa, synthetic construct
LRIKGKYALPVMVNHQHLPTTIPAGSNSYQPVPITIPKESQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 8 - protein, 245 aa, synthetic construct
LRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELASTQPAFSAGPPADDSSSTSSGEWYQNFQPPPQPPSEEQFGCPGSPSPQPDSTDNDDYDDISAA

SEQ ID NO: 9 - protein, 17 aa, synthetic construct
MWLFFGITGLLTAALSG

SEQ ID NO: 10 - protein, 97 aa, synthetic construct
WRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCS

SEQ ID NO: 11 - protein, 41 aa, synthetic construct
ASRSLHNLSTPEVPASVQTVTIESSVTVKIENKESRELMLL

SEQ ID NO: 12 - protein, 21 aa, synthetic construct
IPSIVLGILLLGSLIFIAFIL

SEQ ID NO: 13 - protein, 23 aa, synthetic construct
HPSPAPPDQLNTSSAESELWEPG

SEQ ID NO: 14 - protein, 6 aa, synthetic construct
CSEHQS

SEQ ID NO: 15 - protein, 171 aa, synthetic construct
FQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSD

SEQ ID NO: 16 - protein, 240 aa, synthetic construct
EDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 17 - protein, 4 aa, synthetic construct
ERLP

SEQ ID NO: 18 - protein, 112 aa, synthetic construct
VRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCA

SEQ ID NO: 19 - protein, 4 aa, synthetic construct
ENRA

SEQ ID NO: 20 - protein, 100 aa, synthetic construct
LRLVDGGGACAGRVEMLEHGEWGSVCDDTWDLEDAHVVCRQLGCGWAVQALPGLHFMPGRGPIHRDQVNCSGAEAYLWDCPGLPGQHYCGHKEDAGVVCS

SEQ ID NO: 21 - protein, 421 aa, synthetic construct
MWLFFGITGLLTAALSGWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 22 - protein, 450 aa, synthetic construct
MWLFFGITGLLTAALSGHPSPAPPDQLNTSSAESELWEPGCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 23 - protein, 621 aa, synthetic construct
MWLFFGITGLLTAALSGHPSPAPPDQLNTSSAESELWEPGCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 24 - protein, 690 aa, synthetic construct
MWLFFGITGLLTAALSGHPSPAPPDQLNTSSAESELWEPGCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLEDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 25 - protein, 567 aa, synthetic construct
MWLFFGITGLLTAALSGHPSPAPPDQLNTSSAESELWEPGERLPVRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCAENRALRLVDGGGACAGRVEMLEHGEWGSVCDDTWDLEDAHVVCRQLGCGWAVQALPGLHFMPGRGPIHRDQVNCSGAEAYLWDCPGLPGQHYCGHKEDAGVVCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELASTQPAFSAGPPADDSSSTSSGEWYQNFQPPPQPPSEEQFGCPGSPSPQPDSTDNDDYDDISAA

SEQ ID NO: 26 - protein, 225 aa, synthetic construct
MDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQ

SEQ ID NO: 27 - DNA, 2007 bp, synthetic construct
  source 1..2007: mol_type = other DNA
atgtggctcttcttcgggatcactggattgctgacggcagccctctcaggtcatccatctccagccccacctgaccagctcaacaccagcagtgcagagagtgagctctgggagccaggggagcggcttccggtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgcagagaaccgcgcgctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcacgcccggccgcgggcctatccaccgggaccaggtgaactgctcgggggccgaagcttacctgtgggactgcccggggctgccaggacagcactactgcggccacaaagaggacgcgggcgcggtgtgctcagagcaccagtcctggcgcctgacagggggcgctgaccgctgcgaggggcaggtggaggtacacttccgaggggtctggaacacagtgtgtgacagtgagtggtacccatcggaggccaaggtgctctgccagtccttgggctgtggaactgcggttgagaggcccaaggggctgccccactccttgtccggcaggatgtactactcatgcaatggggaggagctcaccctctccaactgctcctggcggttcaacaactccaacctctgcagccagtcgctggcagccagggtcctctgctcagcttcccggagtttgcacaatctgtccactcccgaagtccctgcaagtgttcagacagtcactatagaatcttctgtgacagtgaaaatagagaacaaggaatctcgggagctaatgctcctcatcccctccatcgttctgggaattctcctccttggctccctcatcttcatagccttcatcctcttgagaattaaaggaaaatatgccctccccgtaatggtgaaccaccagcacctacccaccaccatcccggcagggagcaatagctatcaaccggtccccatcaccatccccaaagaagttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcctag

SEQ ID NO: 28 - DNA, 735 bp, synthetic construct
  source 1..735: mol_type = other DNA
ctgaggatcaagggcaaatacgccctgcctgtgatggtcaaccaccagcatctgcccacaacaatcccagccggcagcaatagctaccagcctgtgcctatcacaatccccaaagaggtgttcatgctgcccatccaggttcaggccccacctcctgaggatagcgatagcggcagcgattccgactacgagcactacgatttcagcgcccagcctcctgtggctctgaccaccttctacaacagccagagacacagagtgaccgacgaggaagtgcagcagagcagattccagatgcctcctctggaagaaggcctggaagaactgcacgcctctcacatccctacagccaatcctggccactgcatcaccgatcctccatctctgggaccccagtatcaccccagaagcaatagcgagtctagcaccagcagcggcgaggactactgcaacagccctaagtctaagctgccaccttggaaccctcaggtgttcagcagcgagagaagcagcttcctggaacagcctcctaacctggaactggccggaacacagcctgccttttctgctggacctcctgccgatgatagcagcagcacatccagcggcgaatggtatcagaacttccagccacctcctcagcctccaagcgaggaacagtttggctgtcctggctctccatcacctcagcctgacagcaccgacaacgacgactacgatgacatcagcgccgct

SEQ ID NO: 29 - DNA, 639 bp, synthetic construct
  source 1..639: mol_type = other DNA
ttgagaattaaaggaaaatatgttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcctag

SEQ ID NO: 30 - DNA, 519 bp, synthetic construct
  source 1..519: mol_type = other DNA
ttgagaattaaaggaaaatatgattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcgggggaggattactgcaatagtcccaaaagcaagccgcctccatggaacccccaggtgttttcttcagagaggagttccttcctggagcagcccccaaacttggagctggccggcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacataggcgcagcctag

SEQ ID NO: 31 - DNA, 537 bp, synthetic construct
  source 1..537: mol_type = other DNA
ttgagaattaaaggaaaatatgttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagggtcccccagccctcagcctgactccaccgacaacgatgactacgatgacatcagcgcagcctag

SEQ ID NO: 32 - DNA, 510 bp, synthetic construct
  source 1..510: mol_type = other DNA
ttgagaattaaaggaaaatatgccctccccgtaatggtgaaccaccagcacctacccaccaccatcccggcagggagcaatagctatcaaccggtccccatcaccatccccaaagaagattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagggtcccccagccctcagcctgactccaccgacaacgatgactacgatgacatcagcgcagcctag

SEQ ID NO: 33 - DNA, 609 bp, synthetic construct
  source 1..609: mol_type = other DNA
ctgaggatcaagggcaaatacgccctgcctgtgatggtcaaccaccagcatctgcccacaacaatccctgccggaagcaacagctaccagcctgtgcctatcacaatccccaaagagtcccagagacaccgcgtgaccgatgaggaagtgcagcagagcagattccagatgcctcctctggaagaaggcctggaagaactgcacgcctctcacatccctacagccaatcctggccactgcatcaccgatcctccatctctgggccctcagtatcaccccagaagcaatagcgagtctagcaccagcagcggcgaggactactgcaacagccctaagtctaagctgcctccatggaatccccaggtgttcagcagcgagagatccagctttctggaacagcctcctaacctggaactggcctctacacagcctgccttttctgccggacctccagccgatgatagcagctctacaagctctggcgaatggtatcagaacttccagcctcctccacagcctccaagcgaggaacagtttggatgtcctggcagcccatctcctcagcctgacagcaccgacaacgacgactacgatgatatcagcgctgcc

SEQ ID NO: 34 - DNA, 735 bp, synthetic construct
  source 1..735: mol_type = other DNA
ctgaggatcaagggcaaatacgccctgcctgtgatggtcaaccaccagcatctgcccacaacaatcccagccggcagcaatagctaccagcctgtgcctatcacaatccccaaagaagtgttcatgctgcccatccaggttcaggccccacctcctgaggatagcgatagcggcagcgattccgactacgagcactacgatttcagcgcccagcctcctgtggctctgaccaccttctacaacagccagagacacagagtgaccgacgaggaagtgcagcagagcagattccagatgcctcctctggaagaaggcctggaagaactgcacgcctctcacatccctacagccaatcctggccactgcatcaccgatcctccatctctgggaccccagtatcaccccagaagcaatagcgagtctagcaccagcagcggcgaggactactgcaacagccctaagtctaagctgccaccttggaaccctcaggtgttcagcagcgagagaagcagctttctcgagcagcctcctaacctggaactggccggaacacagcctgccttttctgctggacctcctgccgatgatagcagcagcacatccagcggcgaatggtatcagaacttccagccacctcctcagcctccaagcgaggaacagtttggctgtcctggctctccatcacctcagcctgacagcaccgacaacgacgactacgatgacatcagcgccgct

SEQ ID NO: 35 - DNA, 735 bp, synthetic construct
  source 1..735: mol_type = other DNA
ctgaggatcaagggcaaatacgccctgccagtgatggtcaaccaccagcatctgcctacaacaatcccagccggcagcaactcttaccagcctgtgccaatcacaatccccaaagaggtgttcatgctgcccatccaggttcaggccccacctcctgaggatagcgatagcggcagcgattccgactacgagcactacgactttagcgcccagcctcctgtggctctgaccaccttctacaacagccagagacacagagtgaccgacgaggaagtgcagcagagcagattccagatgcctcctctggaagaaggcctggaagaactgcacgcctctcacatccctaccgccaatccaggccactgcatcaccgatcctccatctctgggaccccagtatcaccccagaagcaatagcgagagcagcacctctagcggcgaggactactgcaacagccctaagtctaagctgccaccttggaatccccaggtgttcagcagcgagagaagcagctttctggaacagcctcctaacctggaactggccggaacacagcctgccttttctgctggacctcctgccgatgatagcagctctacaagcagcggcgaatggtatcagaacttccagccacctcctcagccacctagcgaagaacagtttggctgtcctggcagcccatctccacagccagacagcaccgacaacgacgactacgatgacatcagcgccgct

SEQ ID NO: 36 - DNA, 735 bp, synthetic construct
  source 1..735: mol_type = other DNA
ttgagaattaaaggaaaatatgccctccccgtaatggtgaaccaccagcacctacccaccaccatcccggcagggagcaatagctatcaaccggtccccatcaccatccccaaagaagttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcgggggaggattactgcaatagtcccaaaagcaagctgcctccatggaacccccaggtgttttcttcagagaggagttccttcctggagcagcccccaaacttggagctggccagcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcc

SEQ ID NO: 37 - DNA, 51 bp, synthetic construct
  source 1..51: mol_type = other DNA
atgtggctgttctttggcatcaccggactgctgacagccgctctgtctgga

SEQ ID NO: 38 - DNA, 51 bp, synthetic construct
  source 1..51: mol_type = other DNA
atgtggctcttcttcgggatcactggattgctgacggcagccctctcaggt

SEQ ID NO: 39 - DNA, 291 bp, synthetic construct
  source 1..291: mol_type = other DNA
tggagactgaccggcggagccgatagatgtgaaggacaggtcgaggtgcacttcagaggcgtgtggaataccgtgtgcgacagcgagtggtatcctagcgaggccaaggtgctgtgtcagtctcttggctgtggaaccgccgtggaaagacctaagggactgcctcacagcctgagcggcagaatgtactacagctgcaacggcgaggaactgaccctgagcaactgcagctggcggttcaacaacagcaacctgtgtagccagagcctggccgccagagtgctgtgttct

SEQ ID NO: 40 - DNA, 291 bp, synthetic construct
  source 1..291: mol_type = other DNA
tggagactgaccggcggagccgatagatgtgaaggacaggtcgaggtgcacttcagaggcgtgtggaacaccgtgtgcgacagcgagtggtatccttctgaggccaaggtgctgtgtcagtctctcggatgtggcaccgccgtggaaagacctaagggactgcctcacagcctgagcggcagaatgtactacagctgcaacggcgaggaactgaccctgagcaactgcagctggcggttcaacaacagcaacctgtgtagccagagcctggccgccagagtgctgtgttct

SEQ ID NO: 41 - DNA, 123 bp, synthetic construct
  source 1..123: mol_type = other DNA
gccagcagaagcctgcacaacctgagcacacctgaagtgcctgccagcgtgcagacagtgacaatcgagagcagcgtgaccgtgaagatcgagaacaaagaaagccgcgagctgatgctgctg

SEQ ID NO: 42 - DNA, 123 bp, synthetic construct
  source 1..123: mol_type = other DNA
gcttcccggagtttgcacaatctgtccactcccgaagtccctgcaagtgttcagacagtcactatagaatcttctgtgacagtgaaaatagagaacaaggaatctcgggagctaatgctcctc

SEQ ID NO: 43 - DNA, 63 bp, synthetic construct
  source 1..63: mol_type = other DNA
atcccttctatcgtgctgggcatcctgctgctgggctccctgatctttatcgcctttatcctg

SEQ ID NO: 44 - DNA, 63 bp, synthetic construct
  source 1..63: mol_type = other DNA
atccctagcatcgtgctgggaatcctgctgctgggcagcctgatctttatcgccttcatcctg

SEQ ID NO: 45 - DNA, 63 bp, synthetic construct
  source 1..63: mol_type = other DNA
atccctagcatcgtgctgggcatcctgctgctgggctccctgatctttatcgcctttatcctg

SEQ ID NO: 46 - DNA, 63 bp, synthetic construct
  source 1..63: mol_type = other DNA
atcccctccatcgttctgggaattctcctccttggctccctcatcttcatagccttcatcctc

SEQ ID NO: 47 - DNA, 69 bp, synthetic construct
  source 1..69: mol_type = other DNA
catccttctccagctccacctgaccagctgaataccagctctgccgagtctgagctgtgggagcctgga

SEQ ID NO: 48 - DNA, 69 bp, synthetic construct
  source 1..69: mol_type = other DNA
catccatctccagccccacctgaccagctcaacaccagcagtgcagagagtgagctctgggagccaggg

SEQ ID NO: 49 - DNA, 18 bp, synthetic construct
  source 1..18: mol_type = other DNA
tgttctgagcaccagtct

SEQ ID NO: 50 - DNA, 513 bp, synthetic construct
  source 1..513: mol_type = other DNA
ttccagaaagccagctccatcgtgtacaagaaagagggcgagcaggtcgagttcagcttccctctggccttcaccgtggaaaagctgacaggatctggcgaactgtggtggcaggccgaaagagcctctagcagcaagagctggatcaccttcgacctgaaaaacaaagaggtgtccgtcaagcgcgtgacacaggaccctaagctgcagatgggaaagaagctgcccctgcacctgacactgcctcaggctttgcctcagtatgccggctctggaaatctgacactggccctggaagccaagaccggcaaactgcatcaagaagtgaacctggtcgtcatgcgggccacacagctgcagaagaatctgacctgcgaagtgtggggccctacaagccctaaactgatgctgagcctgaagctggaaaacaaagaagccaaggtttccaagcgcgagaaggccgtgtgggtgttgaatcctgaagccggcatgtggcagtgcctgctgagcgat

SEQ ID NO: 51 - DNA, 720 bp, synthetic construct
  source 1..720: mol_type = other DNA
gaagatcctgccgagcctaagagccctgacaagacccacacatgccctccatgtcctgctccagaactgctcggcggaccctccgttttcctgtttccacctaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagccaagagaaccccaggtttacaccctgcctccaagcagagatgagctgaccaagaaccaggtgtccctgacctgcctggtcaagggcttctaccctagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaatcactacacccagaagtccctgtctctgagcccaggcaagaaggaccctaag

SEQ ID NO: 52 - DNA, 12 bp, synthetic construct
  source 1..12: mol_type = other DNA
gagcggcttccg

SEQ ID NO: 53 - DNA, 336 bp, synthetic construct
  source 1..336: mol_type = other DNA
gtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgca

SEQ ID NO: 54 - DNA, 12 bp, synthetic construct
  source 1..12: mol_type = other DNA
gagaaccgcgcg

SEQ ID NO: 55 - DNA, 300 bp, synthetic construct
  source 1..300: mol_type = other DNA
ctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcatgcccggccgcgggcctatccaccgggaccaggtgaactgctcgggggccgaagcttacctgtgggactgcccggggctgccaggacagcactactgcggccacaaagaggacgcgggcgtggtgtgctca

SEQ ID NO: 56 - DNA, 13 bp, synthetic construct
  source 1..13: mol_type = other DNA
cctctagactgcc

SEQ ID NO: 57 - DNA, 1263 bp, synthetic construct
  source 1..1263: mol_type = other DNA
atgtggctgttcttcggcatcaccggactgctgacagccgctttatctggatggcgactgaccggcggagccgatagatgtgaaggacaggtggaagtgcactttcggggcgtgtggaacaccgtgtgcgacagcgagtggtatcctagcgaggccaaggtgctgtgtcagagcctgggatgtggcaccgccgtggaaagacctaagggactgcctcacagcctgagcggcagaatgtactacagctgcaacggcgaggaactgaccctgagcaattgcagctggcggttcaacaacagcaacctgtgcagccagtctctggccgccagagtgctgtgtagcgccagcagaagcctgcacaatctgagcacacctgaggtgccagccagcgtgcagacagtgacaatcgagagcagcgtgaccgtgaagatcgagaacaaagaaagccgcgagctgatgctgctgatccccagcatcgtgctgggaatcctgctgctgggcagcctgatctttatcgccttcatcctgctgcggatcaagggcaaatacgccctgcccgtgatggtcaaccaccagcatctgcctacaaccatccctgccggcagcaacagctaccagcctgtgcccatcaccatccccaaagaggtgttcatgctgcccatccaggtgcaggcccctccacctgaggatagcgatagcggcagcgacagcgactacgagcactacgatttttccgcccagcctcctgtggccctgaccaccttctacaacagccagagacacagagtgaccgacgaggaagtgcagcagagcagattccagatgccccctctggaagagggcctggaagaactgcatgccagccacatccctaccgccaatcctggccactgcatcaccgatcctccaagcctgggaccccagtaccaccccagaagcaatagcgagagcagcaccagcagcggcgaggactactgcaacagccctaagagcaagctgcccccctggaaccctcaggtgttcagcagcgagagaagcagcttcctggaacagccccccaatctggaactggccggaacacagcctgccttttctgccggacctcctgccgatgatagcagcagcacaagctccggggagtggtatcagaacttccagcctcctccacagccccctagcgaggaacagtttggctgtcctggaagccccagccctcagcccgatagcaccgacaacgacgactacgacgatatcagcgccgct

SEQ ID NO: 58 - DNA, 1350 bp, synthetic construct
  source 1..1350: mol_type = other DNA
atgtggctgttctttggcatcaccggactgctgacagccgctctgtctggacatccttctccagctccacctgaccagctgaataccagctctgccgagtctgagctgtgggagcctggatgttctgagcaccagtcttggagactgaccggcggagccgatagatgtgaaggacaggtcgaggtgcacttcagaggcgtgtggaataccgtgtgcgacagcgagtggtatcctagcgaggccaaggtgctgtgtcagtctcttggctgtggaaccgccgtggaaagacctaagggactgcctcacagcctgagcggcagaatgtactacagctgcaacggcgaggaactgaccctgagcaactgcagctggcggttcaacaacagcaacctgtgtagccagagcctggccgccagagtgctgtgttctgccagcagaagcctgcacaacctgagcacacctgaagtgcctgccagcgtgcagacagtgacaatcgagagcagcgtgaccgtgaagatcgagaacaaagaaagccgcgagctgatgctgctgatcccttctatcgtgctgggcatcctgctgctgggctccctgatctttatcgcctttatcctgctgaggatcaagggcaaatacgccctgcctgtgatggtcaaccaccagcatctgcccacaacaatcccagccggcagcaatagctaccagcctgtgcctatcacaatccccaaagaggtgttcatgctgcccatccaggttcaggccccacctcctgaggatagcgatagcggcagcgattccgactacgagcactacgatttcagcgcccagcctcctgtggctctgaccaccttctacaacagccagagacacagagtgaccgacgaggaagtgcagcagagcagattccagatgcctcctctggaagaaggcctggaagaactgcacgcctctcacatccctacagccaatcctggccactgcatcaccgatcctccatctctgggaccccagtatcaccccagaagcaatagcgagtctagcaccagcagcggcgaggactactgcaacagccctaagtctaagctgccaccttggaaccctcaggtgttcagcagcgagagaagcagcttcctggaacagcctcctaacctggaactggccggaacacagcctgccttttctgctggacctcctgccgatgatagcagcagcacatccagcggcgaatggtatcagaacttccagccacctcctcagcctccaagcgaggaacagtttggctgtcctggctctccatcacctcagcctgacagcaccgacaacgacgactacgatgacatcagcgccgct

SEQ ID NO: 59 - DNA, 1863 bp, synthetic construct
  source 1..1863: mol_type = other DNA
atgtggctgttctttggcatcaccggactgctgacagccgctctgtctggacatccttctccagctccacctgaccagctgaataccagctctgccgagtctgagctgtgggagcctggatgttctgagcaccagtcttggagactgaccggcggagccgatagatgtgaaggacaggtcgaggtgcacttcagaggcgtgtggaataccgtgtgcgacagcgagtggtatcctagcgaggccaaggtgctgtgtcagtctcttggctgtggaaccgccgtggaaagacctaagggactgcctcacagcctgagcggcagaatgtactacagctgcaacggcgaggaactgaccctgagcaactgcagctggcggttcaacaacagcaacctgtgtagccagagcctggccgccagagtgctgtgttctgccagcagaagcctgcacaacctgagcacacctgaagtgcctgccagcgtgcagacagtgacaatcgagagcagcgtgaccgtgaagatcgagaacaaagaaagccgcgagctgatgctgctgttccagaaagccagctccatcgtgtacaagaaagagggcgagcaggtcgagttcagcttccctctggccttcaccgtggaaaagctgacaggatctggcgaactgtggtggcaggccgaaagagcctctagcagcaagagctggatcaccttcgacctgaaaaacaaagaggtgtccgtcaagcgcgtgacacaggaccctaagctgcagatgggaaagaagctgcccctgcacctgacactgcctcaggctttgcctcagtatgccggctctggaaatctgacactggccctggaagccaagaccggcaaactgcatcaagaagtgaacctggtcgtcatgcgggccacacagctgcagaagaatctgacctgcgaagtgtggggccctacaagccctaaactgatgctgagcctgaagctggaaaacaaagaagccaaggtttccaagcgcgagaaggccgtgtgggtgttgaatcctgaagccggcatgtggcagtgcctgctgagcgatatccctagcatcgtgctgggaatcctgctgctgggcagcctgatctttatcgccttcatcctgctgaggatcaagggcaaatacgccctgcctgtgatggtcaaccaccagcatctgcccacaacaatcccagccggcagcaatagctaccagcctgtgcctatcacaatccccaaagaagtgttcatgctgcccatccaggttcaggccccacctcctgaggatagcgatagcggcagcgattccgactacgagcactacgatttcagcgcccagcctcctgtggctctgaccaccttctacaacagccagagacacagagtgaccgacgaggaagtgcagcagagcagattccagatgcctcctctggaagaaggcctggaagaactgcacgcctctcacatccctacagccaatcctggccactgcatcaccgatcctccatctctgggaccccagtatcaccccagaagcaatagcgagtctagcaccagcagcggcgaggactactgcaacagccctaagtctaagctgccaccttggaaccctcaggtgttcagcagcgagagaagcagctttctcgagcagcctcctaacctggaactggccggaacacagcctgccttttctgctggacctcctgccgatgatagcagcagcacatccagcggcgaatggtatcagaacttccagccacctcctcagcctccaagcgaggaacagtttggctgtcctggctctccatcacctcagcctgacagcaccgacaacgacgactacgatgacatcagcgccgct

SEQ ID NO: 60 - DNA, 2070 bp, synthetic construct
  source 1..2070: mol_type = other DNA
atgtggctgttctttggcatcaccggactgctgacagccgctctgtctggacatccttctccagctccacctgaccagctgaataccagctctgccgagtctgagctgtgggagcctggatgttctgagcaccagtcttggagactgaccggcggagccgatagatgtgaaggacaggtcgaggtgcacttcagaggcgtgtggaacaccgtgtgcgacagcgagtggtatccttctgaggccaaggtgctgtgtcagtctctcggatgtggcaccgccgtggaaagacctaagggactgcctcacagcctgagcggcagaatgtactacagctgcaacggcgaggaactgaccctgagcaactgcagctggcggttcaacaacagcaacctgtgtagccagagcctggccgccagagtgctgtgttctgccagcagaagcctgcacaacctgtctacacctgaggtgccagcctctgtgcagaccgtgacaatcgagtctagcgtgaccgtgaagatcgagaacaaagaaagccgcgagctgatgctgctggaagatcctgccgagcctaagagccctgacaagacccacacatgccctccatgtcctgctccagaactgctcggcggaccctccgttttcctgtttccacctaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagccaagagaaccccaggtttacaccctgcctccaagcagagatgagctgaccaagaaccaggtgtccctgacctgcctggtcaagggcttctaccctagcgacattgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgttctgtgatgcacgaggccctgcacaatcactacacccagaagtccctgtctctgagcccaggcaagaaggaccctaagatccctagcatcgtgctgggcatcctgctgctgggctccctgatctttatcgcctttatcctgctgaggatcaagggcaaatacgccctgccagtgatggtcaaccaccagcatctgcctacaacaatcccagccggcagcaactcttaccagcctgtgccaatcacaatccccaaagaggtgttcatgctgcccatccaggttcaggccccacctcctgaggatagcgatagcggcagcgattccgactacgagcactacgactttagcgcccagcctcctgtggctctgaccaccttctacaacagccagagacacagagtgaccgacgaggaagtgcagcagagcagattccagatgcctcctctggaagaaggcctggaagaactgcacgcctctcacatccctaccgccaatccaggccactgcatcaccgatcctccatctctgggaccccagtatcaccccagaagcaatagcgagagcagcacctctagcggcgaggactactgcaacagccctaagtctaagctgccaccttggaatccccaggtgttcagcagcgagagaagcagctttctggaacagcctcctaacctggaactggccggaacacagcctgccttttctgctggacctcctgccgatgatagcagctctacaagcagcggcgaatggtatcagaacttccagccacctcctcagccacctagcgaagaacagtttggctgtcctggcagcccatctccacagccagacagcaccgacaacgacgactacgatgacatcagcgccgct

SEQ ID NO: 61 - DNA, 1701 bp, synthetic construct
  source 1..1701: mol_type = other DNA
atgtggctcttcttcgggatcactggattgctgacggcagccctctcaggtcatccatctccagccccacctgaccagctcaacaccagcagtgcagagagtgagctctgggagccaggggagcggcttccggtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgcagagaaccgcgcgctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcatgcccggccgcgggcctatccaccgggaccaggtgaactgctcgggggccgaagcttacctgtgggactgcccggggctgccaggacagcactactgcggccacaaagaggacgcgggcgtggtgtgctcagcttcccggagtttgcacaatctgtccactcccgaagtccctgcaagtgttcagacagtcactatagaatcttctgtgacagtgaaaatagagaacaaggaatctcgggagctaatgctcctcatcccctccatcgttctgggaattctcctccttggctccctcatcttcatagccttcatcctcttgagaattaaaggaaaatatgccctccccgtaatggtgaaccaccagcacctacccaccaccatcccggcagggagcaatagctatcaaccggtccccatcaccatccccaaagaagttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcgggggaggattactgcaatagtcccaaaagcaagctgcctccatggaacccccaggtgttttcttcagagaggagttccttcctggagcagcccccaaacttggagctggccagcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcc

SEQ ID NO: 62 - DNA, 675 bp, synthetic construct
  source 1..675: mol_type = other DNA
ggcgacaacaccgaggacgtgatcaaagaattcatgcagttcaaagtgcggatggaaggcagcgtgaacggccactacttcgagattgaaggcgaaggcgagggcaagccttacgagggaacacagacagccaagctgcaagtgacaaaaggcggccctctgccttttgcctgggacattctgagccctcagttccagtacggcagcaaggcctatgtgaagcaccccgccgacattcccgactacatgaagctgagcttccccgagggcttcacctgggagagatccatgaatttcgaggacggcggcgtggtggaagtccagcaagatagttctctgcaggacggcaccttcatctacaaagtgaagttcaagggcgtgaacttccccgctgatggccccgtgatgcagaaaaagacagccggatgggagccctccaccgagaagctttatcctcaagatggcgtgctgaagggcgagatctctcacgccctgaagctgaaggatggcggccactatacctgcgacttcaagaccgtgtacaaggccaagaaacccgtgcagctgcccggcaaccactacgtggacagcaagctggacatcaccaaccacaacgaggattacacagtggtggaacagtacgagcacgccgaggccagacactctggatctcag

SEQ ID NO: 63 - protein, 5 aa, synthetic construct
GSGGS

SEQ ID NO: 64 - protein, 4 aa, synthetic construct
GGGS

SEQ ID NO: 65 - protein, 4 aa, synthetic construct
GGSG

SEQ ID NO: 66 - protein, 5 aa, synthetic construct
GGSGG

SEQ ID NO: 67 - protein, 5 aa, synthetic construct
GSGSG

SEQ ID NO: 68 - protein, 5 aa, synthetic construct
GSGGG

SEQ ID NO: 69 - protein, 5 aa, synthetic construct
GGGSG

SEQ ID NO: 70 - protein, 5 aa, synthetic construct
GSSSG

SEQ ID NO: 71 - protein, 5 aa, synthetic construct
GGGGS